(19) United States
(12) Patent Application Publication
(10) Pub. No.: US 2014/0173776 A1
(43) Pub. Date: Jun. 19, 2014

(54) Herbicide-Metabolizing Cytochrome P450 Monooxygenases

(71) Applicant: BASF SE, Ludwigshafen (DE)

(72) Inventors: Stefan TRESCH, Kirchheim (DE); Doreen SCHACHTSCHABEL, Mannheim (DE); Maciej PASTERNAK, Ludwigshafen (DE); Liliana PARRA RAPADO, Offenburg (DE); Jens LERCHL, Golm (DE); Thomas MIETZNER, Annweiler (DE); Martin LaFOREST, St.-Jean-sur-Richelieu (CA)

(73) Assignee: BASF SE, Ludwigshafen (DE)

(21) Appl. No.: 14/013,825

(22) Filed: Aug. 29, 2013

Related U.S. Application Data
(60) Provisional application No. 61/738,437, filed on Dec. 18, 2012.

(51) Int. Cl. C12N 15/82

(52) U.S. Cl. CPC C12N 15/8274
USPC 800/278; 536/23.2; 504/196; 435/320.1; 435/189; 435/418; 435/413; 435/415; 435/417; 435/416; 435/414; 800/300; 435/29

(57) ABSTRACT

The present invention refers to method for producing a transgenic plant with increased herbicide tolerance or resistance as compared to a corresponding non-transformed wild type plant, comprising transforming a plant cell or a plant cell nucleus or a plant tissue with a nucleic acid molecule encoding an Alopecurus cytochrome P450 monooxygenase, as well as to the nucleic acid, and plants with increased herbicide tolerance or resistance comprising the nucleic acid of the invention.

This application claims priority of application with No. 61/738,437, which is incorporated by reference in its entirety.

### FIELD OF THE INVENTION

The present invention relates to Alopecurus cytochrome P450 monooxygenases which are able to metabolize herbicides such as saflufenacil, as well as polynucleotides encoding this enzyme. The invention also relates to transgenic plants producing these enzymes which are resistant and/or tolerant to herbicide activity, when treated therewith.

### BACKGROUND OF THE INVENTION

Cytochrome P450 monooxygenases (hereinafter “CytP450s”) form a large diverse gene family with about 246 isoforms in Arabidopsis and 372 identified in rice. CytP450s are hemoproteins that convert a broad range of substrates to more or less bioactive products. The reaction cycle catalyzed by CytP450s requires the sequential input of two reducing equivalents (i.e., two electrons and two protons). The reducing equivalents for the CytP450-catalyzed reaction are supplied by either NADPH or NADH, depending on the type of redox system concerned, and electron transfer is mediated by two co-factors, one of which is FAD; the other being either FMN or an iron-sulfur Fe₂S₂ redoxin (ferredoxin) or, in the microsomal system, cytochrome b5. In particular, the majority of plant CytP450s utilize an electron transport chain which consists of an FAD-containing NADPH-dependent oxidoreductase (Werck-Reichhart, Trends in plant science 5 (2000) 116-123). The mitochondrial system in mammalia bears many similarities with the plant P450 electron transport chain and both systems are generally referred to as Class I (see Lewis and Hlavica, Biochimica et Biophysica Acta 1460 (2000) 353-374, as well as references contained therein). CytP450s are critical in numerous plant metabolic pathways, including biosynthesis of hormones, secondary metabolites and lipids, particularly lignin and pigment biosynthesis, detoxification of harmful compounds, and are considered important in the evolution of land plants. Inhibitors of CytP450 activity include 1-aminobenzo-triazole, tetcyclacis, piperonyl butoxide, cinnamonic acid, and tridiphane.

Several approaches can lead to herbicide tolerant plants: a) modification of the target molecule of the herbicide, b) metabolic approach, i.e. making the compound non-hazardous. For the metabolic solution, one or more enzymes are needed, that catalyze the conversion of the herbicide to a non toxic compound. One source of such enzymes can be microorganisms isolated from nature. Bacteria, especially those of the order Actinomycetales, are known for their potential to detoxify soil by metabolizing xenobiotics, including herbicides (Cork et al, 1991; Schrijver et al., 1999; Caracciolo et al., 2010). These detoxifying reactions can be catalyzed by O-demethylases from Pseudomonas maltophilia DI-6, like it was shown for the herbicide Dicamba (Chakraborty et al., 2005; Wang et al., 1997).

In many other cases those reactions are catalyzed by CytP450's. Those can be plant derived (Pan et al., 2006), from algal (Thies et al., 1996) or microbial origin (O'Keefe et al., 1991). Among bacteria, especially actinomycetes offer a broad spectrum of CytP450's. The genome analysis of Streptomyes coelicolor revealed 18 CytP450's (Lamb et al., 2002), the Streptomyes avermitilis genome revealed 33 (Lamb et al., 2003), respectively. According to Nelson (2011), actinobacteria hold the largest number of CytP450's per genome.

Current enzymes available for metabolizing herbicides, such as, for example, saflufenacil, e.g. those described in WO2010/143743 particularly when expressed in plants, do not have particularly high activity. Thus, there is the need for the identification of further enzymes which can be used to metabolize herbicides.

The present inventors have characterized an Alopecurus species utilizing a mechanism of metabolizing herbicides. Furthermore, the inventors have isolated and characterized the novel herbicide-metabolizing CytP450 monooxygenases from this grass species.

### KEY TO SEQUENCE LISTING

| Name | SEQ ID NO | type | SEQ ID NO | type |
|---|---|---|---|---|
| Am_CYP01 | 1 | nucleic acid | 2 | amino acid |
| Am_CYP03 | 3 | nucleic acid | 4 | amino acid |
| Am_CYP03b | 5 | nucleic acid | 6 | amino acid |
| Am_CYP15 | 7 | nucleic acid | 8 | amino acid |
| Q9ATV4 | | | 9 | amino acid |
| B9F5T6 | | | 10 | amino acid |
| Q94HA6 | | | 11 | amino acid |
| Q9ATV6 | | | 12 | amino acid |
| B9F5T8 | | | 13 | amino acid |
| C0KHM1 | | | 14 | amino acid |
| Q94HA5 | | | 15 | amino acid |
| F2EH65 | | | 16 | amino acid |
| Q2LA61 | | | 17 | amino acid |
| F2DYW1 | | | 18 | amino acid |
| Q6F4F4 | | | 19 | amino acid |
| Q6F4F3 | | | 20 | amino acid |
| Q6F4F2 | | | 21 | amino acid |
| F2DH14 | | | 22 | amino acid |
| Q9ATV5 | | | 23 | amino acid |
| Q0DND2 | | | 24 | amino acid |
| B6SSF2 | | | 25 | amino acid |
| Am_CYP04 | 26 | nucleic acid | 27 | amino acid |
| Q9ATU5 | | | 28 | amino acid |
| Q8LL74 | | | 29 | amino acid |
| Q9FDZ1 | | | 30 | amino acid |
| Q9AX23 | | | 31 | amino acid |
| Q6I5Q4 | | | 32 | amino acid |
| A2WUP8 | | | 33 | amino acid |
| Q9ATU3 | | | 34 | amino acid |
| B9FP88 | | | 35 | amino acid |
| Q9ATU2 | | | 36 | amino acid |
| C5XEE4 | | | 37 | amino acid |
| C5XEE3 | | | 38 | amino acid |
| Q9ATU4 | | | 39 | amino acid |
| Q8LGM8 | | | 40 | amino acid |
| C4J0D4 | | | 41 | amino acid |
| Q9ATU1 | | | 42 | amino acid |
| B8AC00 | | | 43 | amino acid |
| Am_CYP12 | 44 | nucleic acid | 45 | amino acid |
| B8B662 | | | 46 | amino acid |
| B6SSZ4 | | | 47 | amino acid |
| B4FMT5 | | | 48 | amino acid |
| C5X3A1 | | | 49 | amino acid |
| B9FWV1 | | | 50 | amino acid |
| Q6T485 | | | 51 | amino acid |
| A3BMK1 | | | 52 | amino acid |
| B8A062 | | | 53 | amino acid |
| C5X3A3 | | | 54 | amino acid |
| Q8L4Q4 | | | 55 | amino acid |
| B8B554 | | | 56 | amino acid |
| Q8LIR5 | | | 57 | amino acid |
| B9FUF2 | | | 58 | amino acid |
| B8B553 | | | 59 | amino acid |
| Q8LHV0 | | | 60 | amino acid |
| F2DQ95 | | | 61 | amino acid |
| C4JB42 | | | 62 | amino acid |
| Q0D4C4 | | | 63 | amino acid |
| B8ARP1 | | | 64 | amino acid |
| F2DL54 | | | 65 | amino acid |
| Q6DV71 | | | 66 | amino acid |
| Q0D4C5 | | | 67 | amino acid |
| A2YP19 | | | 68 | amino acid |
| C5X3A2 | | | 69 | amino acid |
| Am_CYP01 | 70 | Nucleic acid | | |
| Am_CYP03 | 71 | Nucleic acid | | |
| Am_CYP03b | 72 | Nucleic acid | | |
| Am_CYP04 | 73 | Nucleic acid | | |
| Am_CYP12 | 74 | Nucleic acid | | |

### SUMMARY OF THE INVENTION

Accordingly, in one embodiment, the present invention provides a method for producing a plant having an increased herbicide tolerance or resistance as compared to a corresponding wild type plant whereby the method comprises at least the following step: increasing or generating in a plant the activity of an Alopecurus CytP450, or a homolog thereof.

Accordingly, the invention provides a transgenic plant that over-expresses an isolated Alopecurus CytP450 polynucleotide, or a homolog thereof, in the sub-cellular compartment and tissue as indicated herein. The transgenic plant of the invention demonstrates an improved or increased herbicide tolerance or resistance as compared to a wild type variety of the plant.

Accordingly, the invention provides a method for producing a plant with increased herbicide tolerance or resistance as compared to a corresponding wild type plant comprising at least one of the steps selected from the group consisting of: (i) increasing or generating the activity of a polypeptide comprising at least one polypeptide motif or consensus sequence comprising the sequence of SEQ ID NO: 2, 4, 6, 8, 27, or 45, or a homolog thereof; or (ii) increasing or generating the activity of an expression product of one or more isolated polynucleotide(s) comprising one or more polynucleotide(s) comprising the sequence of SEQ ID NO: 1, 3, 5, 7, 26, or 44, or a homolog thereof.

The invention further provides a method for increasing herbicide tolerance or resistance of a crop plant, the method comprising the following steps: (i) increasing or generating of the expression of at least one polynucleotide; and/or (ii) increasing or generating the expression of an expression product encoded by at least one polynucleotide; and/or (iii) increasing or generating one or more activities of an expression product encoded by at least one polynucleotide, wherein the polynucleotide is selected from the group consisting of:
(a) an isolated polynucleotide encoding the polypeptide comprising the sequence of SEQ ID NO: 2, 4, 6, 8, 27, or 45, or a homolog thereof;
(b) an isolated polynucleotide comprising the sequence of SEQ ID NO: 1, 3, 5, 7, 26, or 44, or a homolog thereof;
(c) an isolated polynucleotide, which, as a result of the degeneracy of the genetic code, can be derived from a polypeptide comprising the sequence of SEQ ID NO: 2, 4, 6, 8, 27, or 45, or a homolog thereof and confers an increased herbicide tolerance or resistance as compared to a corresponding, e.g. non-transformed, wild type plant cell, a transgenic plant or a part thereof;
(d) an isolated polynucleotide having 30 or more, for example 50%, 60%, 70%, 80%, 85%, 90%, 95%, 97%, 98%, or 99% (percent) or more identity with the sequence of a polynucleotide comprising the sequence of SEQ ID NO: 1, 3, 5, 7, 26, or 44, or a homolog thereof and conferring an increased herbicide tolerance or resistance as compared to a corresponding, e.g. non-transformed, wild type plant cell, a transgenic plant or a part thereof;
(e) an isolated polynucleotide encoding a polypeptide having 30 or more, for example 50%, 60%, 70%, 80%, 85%, 90%, 95%, 97%, 98%, or 99% or more identity with the amino acid sequence of the polypeptide encoded by the isolated polynucleotide of (a) to (c) and conferring an increased herbicide tolerance or resistance as compared to a corresponding, e.g. non-transformed, wild type plant cell, a transgenic plant or a part thereof;
(f) an isolated polynucleotide which hybridizes with an isolated polynucleotide of (a) to (c) under stringent hybridization conditions and confers an increased herbicide tolerance or resistance as compared to a corresponding, e.g. non-transformed, wild type plant cell, a transgenic plant or a part thereof;
(g) an isolated polynucleotide encoding a polypeptide which can be isolated with the aid of monoclonal or polyclonal antibodies made against a polypeptide encoded by one of the isolated polynucleotides of (a) to (e) and which has the activity represented by the polynucleotide comprising the sequence of SEQ ID NO: 1, 3, 5, 7, 26, or 44, or a homolog thereof;

Furthermore, the invention relates to a method for producing a transgenic plant with increased herbicide tolerance or resistance as compared to a corresponding, e.g. non-transformed, wild type plant, comprising transforming a plant cell or a plant cell nucleus or a plant tissue to produce such a plant, with an isolated polynucleotide selected from the group consisting of:
(a) an isolated polynucleotide encoding the polypeptide comprising the sequence of SEQ ID NO: 2, 4, 6, 8, 27, or 45, or a homolog thereof;
(b) an isolated polynucleotide comprising the sequence of SEQ ID NO: 1, 3, 5, 7, 26, or 44, or a homolog thereof;
(c) an isolated polynucleotide, which, as a result of the degeneracy of the genetic code, can be derived from a polypeptide comprising the sequence of SEQ ID NO: 2, 4, 6, 8, 27, or 45, or a homolog thereof and confers an increased herbicide tolerance or resistance as compared to a corresponding, e.g. non-transformed, wild type plant cell, a transgenic plant or a part thereof;
(d) an isolated polynucleotide having 30 or more, for example 50%, 60%, 70%, 80%, 85%, 90%, 95%, 97%, 98%, or 99% (percent) or more identity with the sequence of a polynucleotide comprising the sequence of SEQ ID NO: 1, 3, 5, 7, 26, or 44, or a homolog thereof and conferring an increased herbicide tolerance or resistance as compared to a corresponding, e.g. non-transformed, wild type plant cell, a transgenic plant or a part thereof;
(e) an isolated polynucleotide encoding a polypeptide having 30 or more, for example 50%, 60%, 70%, 80%, 85%, 90%, 95%, 97%, 98%, or 99% or more identity with the amino acid sequence of the polypeptide encoded by the isolated polynucleotide of (a) to (c) and conferring an increased herbicide tolerance or resistance as compared to a corresponding, e.g. non-transformed, wild type plant cell, a transgenic plant or a part thereof;
(f) an isolated polynucleotide which hybridizes with an isolated polynucleotide of (a) to (c) under stringent hybridization conditions and confers an increased herbicide tolerance or resistance as compared to a corresponding, e.g. non-transformed, wild type plant cell, a transgenic plant or a part thereof;
(g) an isolated polynucleotide encoding a polypeptide which can be isolated with the aid of monoclonal or polyclonal antibodies made against a polypeptide encoded by one of the isolated polynucleotides of (a) to (e) and which has the activity represented by the polynucleotide comprising the sequence of SEQ ID NO: 1, 3, 5, 7, 26, or 44, or a homolog thereof.

### DETAILED DESCRIPTION OF THE PREFERRED EMBODIMENTS

### Definition Collection

An “herbicide tolerance or resistance-increasing activity” according to the invention refers to an activity of a CytP450 from Alopecurus comprising the sequence of SEQ ID NO: 2, 4, 6, 8, 27, or 45, or a homolog thereof. A polypeptide conferring a herbicide tolerance or resistance-increasing activity can be encoded by a nucleic acid sequence comprising the sequence of SEQ ID NO: 1, 3, 5, 7, 26, or 44, or a homolog thereof, and/or comprises or consists of a polypeptide comprising the sequence of SEQ ID NO: 2, 4, 6, 8, 27, or 45, or a homolog thereof.

A “transgenic plant”, as used herein, refers to a plant which contains a foreign nucleotide sequence inserted into either its nuclear genome or organelle genome. It encompasses further the offspring generations i.e. the T1-, T2- and consecutively generations or BC1-, BC2- and consecutively generation as well as crossbreeds thereof with non-transgenic or other transgenic plants.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into a organism, transient or stable. Furthermore such an increase can be reached by the introduction of the inventive nucleic acid sequence or the encoded protein in the correct cell compartment for example into the nucleus or cytoplasmic respectively or into plastids either by transformation and/or targeting.

For the purposes of the description of the present invention, the terms “cytoplasmic” and “non-targeted” shall indicate, that the nucleic acid of the invention is expressed without the addition of a non-natural transit peptide encoding sequence. A non-natural transit peptide encoding sequence is a sequence which is not a natural part of a nucleic acid of the invention, e.g. of the nucleic acids depicted in SEQ ID NO: 1, 3, 5, 7, 26, or 44, or a homolog thereof, but is rather added by molecular manipulation steps which are well known to the person skilled in the art or as for example described hereinafter. Therefore the terms “cytoplasmic” and “non-targeted” shall not exclude a targeted localization to any cell compartment for the products of the inventive nucleic acid sequences by their naturally occurring sequence properties within the background of the transgenic organism. The sub-cellular location of the mature polypeptide derived from the enclosed sequences can be predicted by a skilled person for the organism (plant) by using software tools like TargetP (Emanuelsson et al., (2000), Predicting sub-cellular localization of proteins based on their N-terminal amino acid sequence. J. Mol. Biol. 300, 1005-1016), ChloroP (Emanuelsson et al. (1999), ChloroP, a neural network-based method for predicting chloroplast transit peptides and their cleavage sites. Protein Science, 8: 978-984) or other predictive software tools (Emanuelsson et al. (2007), locating proteins in the cell using TargetP, SignalP, and related tools (Nature Protocols 2, 953-971).

The term “organelle” according to the invention shall mean for example “mitochondria”, “plastid” or endoplasmic reticulum (ER). The term “plastid” according to the invention is intended to include various forms of plastids including proplastids, chloroplasts, chromoplasts, gerontoplasts, leucoplasts, amyloplasts, elaioplasts and etioplasts, preferably chloroplasts. They all have as a common ancestor the aforementioned proplasts.

The term “introduced” in the context of this specification shall mean the insertion of a nucleic acid sequence into the organism by means of a “transfection”, “transduction” or preferably by “transformation”.

A plastid, such as a chloroplast, has been “transformed” by an exogenous (preferably foreign) nucleic acid sequence if nucleic acid sequence has been introduced into the plastid that means that this sequence has crossed the membrane or the membranes of the plastid. The foreign DNA may be integrated (covalently linked) into plastid DNA making up the genome of the plastid, or it may remain not integrated (e.g., by including a chloroplast origin of replication). “Stably” integrated DNA sequences are those, which are inherited through plastid replication, thereby transferring new plastids, with the features of the integrated DNA sequence to the progeny.

As used herein, “plant” is meant to include not only a whole plant but also a part thereof i.e., one or more cells, and tissues, including for example, leaves, stems, shoots, roots, flowers, fruits and seeds.

The term “herbicide tolerance or resistance” as used herein it is intended that a plant that is tolerant or resistant to at least one herbicide at a level that would normally kill, or inhibit the growth of, a normal or wild-type plant.

Any increase in herbicide tolerance or resistance is an improved herbicide tolerance or resistance in accordance with the invention. For example, the improvement in herbicide tolerance or resistance can comprise a 1.5×, 2×, 2.5×, 3×, 5×, 10×, 20×, 30×, 40×, 50×, 75×, 100×, 150×, 200× or greater increase in any measurable parameter.

Generally, the term “herbicide” is used herein to mean an active ingredient that kills, controls or otherwise adversely modifies the growth of plants. The preferred amount or concentration of the herbicide is an “effective amount” or “effective concentration.” By “effective amount” and “effective concentration” is intended an amount and concentration, respectively, that is sufficient to kill or inhibit the growth of a similar, wild-type, plant, plant tissue, plant cell, or host cell, but that said amount does not kill or inhibit as severely the growth of the herbicide-resistant plants, plant tissues, plant cells, and host cells of the present invention. Typically, the effective amount of a herbicide is an amount that is routinely used in agricultural production systems to kill weeds of interest. Such an amount is known to those of ordinary skill in the art. Herbicidal activity is exhibited by the herbicides useful for the present invention when they are applied directly to the plant or to the locus of the plant at any stage of growth or before planting or emergence. The effect observed depends upon the plant species to be controlled, the stage of growth of the plant, the application parameters of dilution and spray drop size, the particle size of solid components, the environmental conditions at the time of use, the specific compound employed, the specific adjuvants and carriers employed, the soil type, and the like, as well as the amount of chemical applied. These and other factors can be adjusted as is known in the art to promote non-selective or selective herbicidal action. Generally, it is preferred to apply the herbicide postemergence to relatively immature undesirable vegetation to achieve the maximum control of weeds.

More specifically, the term “herbicide” is meant to include any molecule that, when exogenously applied to a plant, has a deleterious effect on said plant. Examples of herbicides that are useful for the present invention include saflufenacil, benzoxazinone-derivative, benzobicyclon, mesotrione, sulcotrione, tefuryltrione, tembotrione, 4-hydroxy-3-[[2-(2-methoxyethoxy)methyl]-6-(trifluoromethyl)-3-pyridinyl]carbonyl]-bicyclo[3.2.1]-oct-3-en-2-one (bicyclopyrone), ketospiradox or the free acid thereof, benzofenap, pyrasulfotole, pyrazolynate, pyrazoxyfen, topramezone, [2-chloro-3-(2-methoxyethoxy)-4-(methylsulfonyl)phenyl](l-ethyl-5-hydroxy-1H-pyrazol-4-yl)-methanone, (2,3-dihydro-3,3,4-trimethyl-1,1-dioxidobenzo[b]thien-5-yl)(5-hydroxy-1-methyl-1H-pyrazol-4-yl)-methanone, isoxachlortole, isoxaflutole, α-(cyclopropylcarbonyl)-2-(methylsulfonyl)β-oxo-4-chlorobenzenepropanenitrile, and α-(cyclopropylcarbonyl)-2-(methylsulfonyl)β-oxo-4-(trifluoromethyl)-benzenepropanenitrile, topramezone.

Other herbicidal compounds useful for the present invention may further include herbicides to which the crop plant is naturally tolerant, or to which it is resistant via expression of one or more additional transgenes. Some of the herbicides that can be employed with the CytP450 enzymes the present invention include sulfonamides such as metosulam, flumetsulam, cloransulam-methyl, diclosulam, penoxsulam and florasulam, sulfonylureas such as chlorimuron, tribenuron, sulfometuron, nicosulfuron, chlorsulfuron, amidosulfuron, triasulfuron, prosulfuron, tritosulfuron, thifensulfuron, sulfosulfuron and metsulfuron, imidazolinones such as imazaquin, imazapic, ima-zethapyr, imzapyr, imazamethabenz and imazamox, phenoxyalkanoic acids such as 2,4-D, MCPA, dichlorprop and mecoprop, pyridinyloxyacetic acids such as triclopyr and fluoroxypyr, carboxylic acids such as clopyralid, picloram, aminopyralid and dicamba, dinitroanilines such as trifluralin, benefin, benfluralin and pendimethalin, chloroacetanilides such as alachlor, acetochlor and metolachlor, semicarbazones (auxin transport inhibitors) such as chlorflurenol and diflufenzopyr, aryloxyphenoxypropionates such as fluazifop, haloxyfop, diclofop, clodinafop and fenoxaprop and other common herbicides including glyphosate, glufosinate, acifluorfen, bentazon, clomazone, fumiclorac, fluometuron, fomesafen, lactofen, linuron, isoproturon, simazine, norflurazon, paraquat, diuron, diflufenican, picolinafen, cinidon, sethoxydim, tralkoxydim, quinmerac, isoxaben, bromoxynil, metribuzin and mesotrione, glyphosate, glufosinate.

Unless already included in the disclosure above, the herbicides useful for the present invention can, further, comprise compounds:

(a) from the group of Lipid Biosynthesis Inhibitors:
Alloxydim, Alloxydim-natrium, Butroxydim, Clethodim, Clodinafop, Clodinafop-propargyl, Cycloxydim, Cyhalofop, Cyhalofop-butyl, Diclofop, Diclofop-methyl, Fenoxaprop, Fenoxaprop-ethyl, Fenoxaprop-P, Fenoxaprop-P-ethyl, Fluazifop, Fluazifop-butyl, Fluazifop-P, Fluazifop-P-butyl, Haloxyfop, Haloxyfop-methyl, Haloxyfop-P, Haloxyfop-P-methyl, Metamifop, Pinoxaden, Profoxydim, Propaquizafop, Quizalofop, Quizalofop-ethyl, Quizalofoptefuryl, Quizalofop-P, Quizalofop-P-ethyl, Quizalofop-P-tefuryl, Sethoxydim, Tepraloxydim, Tralkoxydim, Benfuresat, Butylat, Cycloat, Dalapon, Dimepiperat, EPTC, Esprocarb, Ethofumesat, Flupropanat, Molinat, Orbencarb, Pebulat, Prosulfocarb, TCA, Thiobencarb, Tiocarbazil, Triallat and Vernolat;
(b) from the group of Acetohydroxyacid synthase (AHAS) Inhibitors:
Amidosulfuron, Azimsulfuron, Bensulfuron, Bensulfuron-methyl, Bispyribac, Bispyribacnatrium, Chlorimuron, Chlorimuron-ethyl, Chlorsulfuron, Cinosulfuron, Cloransulam, Cloransulam-methyl, Cyclosulfamuron, Diclosulam, Ethametsulfuron, Ethametsulfuron-methyl, Ethoxysulfuron, Flazasulfuron, Florasulam, Flucarbazon, Flucarbazon-natrium, Flucetosulfuron, Flumetsulam, Flupyrsulfuron, Flupyrsulfuron-methyl-natrium, Foramsulfuron, Halosulfuron, Halosulfuron-methyl, Imazamethabenz, Imazamethabenz-methyl, Imazamox, Imazapic, Imazapyr, Imazaquin, Imazethapyr, Imazosulfuron, Iodosulfuron, Iodosulfuronmethyl-natrium, Mesosulfuron, Metosulam, Metsulfuron, Metsulfuron-methyl, Nicosulfuron, Orthosulfamuron, Oxasulfuron, Penoxsulam, Primisulfuron, Primisulfuron-methyl, Propoxycarbazon, Propoxycarbazon-natrium, Prosulfuron, Pyrazosulfuron, Pyrazosulfuronethyl, Pyribenzoxim, Pyrimisulfan, Pyriftalid, Pyriminobac, Pyriminobac-methyl, Pyrithiobac, Pyrithiobac-natrium, Pyroxsulam, Rimsulfuron, Sulfometuron, Sulfometuron-methyl, 5 ulfosulfuron, Thiencarbazon, Thiencarbazon-methyl, Thifensulfuron, Thifensulfuron-methyl, Triasulfuron, Tribenuron, Tribenuron-methyl, Trifloxysulfuron, Triflusulfuron, Triflusulfuronmethyl and Tritosulfuron;
(c) from the group of Photosynthesis-Inhibitors:
Ametryn, Amicarbazon, Atrazin, Bentazon, Bentazon-natrium, Bromacil, Bromofenoxim, Bromoxynil and its salts and esters, Chlorobromuron, Chloridazon, Chlorotoluron, Chloroxuron, Cyanazin, Desmedipham, Desmetryn, Dimefuron, Dimethametryn, Diquat, Diquat-dibromid, Diuron, Fluometuron, Hexazinon, loxynil and its salts and esters, Isoproturon, Isouron, Karbutilat, Lenacil, Linuron, Metamitron, Methabenzthiazuron, Metobenzuron, Metoxuron, Metribuzin, Monolinuron, Neburon, Paraquat, Paraquat-dichlorid, Paraquat-dimetilsulfat, Pentanochlor, Phenmedipham, Phenmedipham-ethyl, Prometon, Prometryn, Propanil, Propazin, Pyridafol, Pyridat, Siduron, Simazin, Simetryn, Tebuthiuron, Terbacil, Terbumeton, Terbuthylazin, Terbutryn, Thidiazuron and Trietazin;
d) from the group of Protoporphyrinogen-IX-Oxidase-Inhibitors:
Acifluorfen, Acifluorfen-natrium, Azafenidin, Bencarbazon, Benzfendizon, Benzoxazinone (as described in WO2010/145992) Bifenox, Butafenacil, Carfentrazon, Carfentrazon-ethyl, Chlomethoxyfen, Cinidon-ethyl, Fluazolat, Flufenpyr, Flufenpyr-ethyl, Flumiclorac, Flumiclorac-pentyl, Flumioxazin, Fluoroglycofen, Fluoroglycofen-ethyl, Fluthiacet, Fluthiacet-methyl, Fomesafen, Halosafen, Lactofen, Oxadiargyl, Oxadiazon, Oxyfluorfen, Pentoxazon, Profluazol, Pyraclonil, Pyraflufen, Pyraflufen-ethyl, Saflufenacil, Sulfentrazon, Thidiazimin, 2-Chlor-5-[3,6-dihydro-3-methyl-2,6-dioxo-4-(trifluormethyl)-1(2H)-pyrimidinyl]-4-fluor-N—[(isopropyl)methylsulfamoyl]benzamid (H-1; CAS 372137-35-4), [3-[2-Chlor-4-fluor-5-(1-methyl-6-trifluormethyl-2,4-dioxo-1,2,3,4,-tetrahydropyrimidin-3-yl)phenoxy]-2-pyridyloxy]acetic acidethylester (H-2; CAS 353292-31-6), N-Ethyl-3-(2,6-dichlor-4-trifluormethylphenoxy)-5-methyl-1H-pyrazol-1-carboxamid (H-3; CAS 452098-92-9), N-Tetrahydrofurfuryl-3-(2,6-dichlor-4-trifluormethylphenoxy)-5-methyl-1H-pyrazol-1-carboxamid (H-4; CAS 915396-43-9), N-Ethyl-3-(2-chlor-6-fluor-4-trifluormethylphenoxy)-5-methyl-1H-pyrazol-1-carboxamid (H-5; CAS 452099-05-7) and N-Tetrahydrofurfuryl-3-(2-chlor-6-fluor-4-trifluormethylphenoxy)-5-methyl-1H-pyrazol-1-carboxamid (H-6; CAS 45100-03-7);
e) from the group of Bleacher-Herbicides:
Aclonifen, Amitrol, Beflubutamid, Benzobicyclon, Benzofenap, Clomazon, Coumarone-derivative herbicides, Diflufenican, Fluridon, Fluorochloridon, Flurtamon, Isoxaflutol, Mesotrion, Norflurazon, Picolinafen, Pyrasulfutol, Pyrazolynat, Pyrazoxyfen, Sulcotrion, Tefuryltrion, Tembotrion, Topramezon, 4-Hydroxy-3-[[2-[(2-methoxyethoxy)methyl]-6-(trifluormethyl)-3-pyridyl]carbonyl]bicyclo[3.2.1]oct-3-en-2-one (H-7; CAS 352010-68-5) and 4-(3-Trifluormethylphenoxy)-2-(4-trifluormethylphenyl)pyrimidin (H-8; CAS 180608-33-7);
f) from the group of 5-enolpyruyylshikimate-3-phosphate-synthase-Inhibitors:

### Glyphosat, Glyphosat-isopropylammonium and Glyphosat-trimesium (Sulfosat);

g) from the group of Glutamin-Synthase-Inhibitors:

### Bilanaphos (Bialaphos), Bilanaphos-natrium, Glufosinat and Glufosinat-ammonium;

h) from the group of DHP-Synthase-Inhibitors: Asulam;
i) from the group of Mitose-Inhibitors:
Amiprophos, Amiprophos-methyl, Benfluralin, Butamiphos, Butralin, Carbetamid, Chlorpropham, Chlorthal, Chlorthal-dimethyl, Dinitramin, Dithiopyr, Ethalfluralin, Fluchloralin, Oryzalin, Pendimethalin, Prodiamin, Propham, Propyzamid, Tebutam, Thiazopyr and Trifluralin;
j) from the group of VLCFA-Inhibitors:
Acetochlor, Alachlor, Anilofos, Butachlor, Cafenstrol, Dimethachlor, Dimethanamid, Dimethenamid-P, Diphenamid, Fentrazamid, Flufenacet, Mefenacet, Metazachlor, Metolachlor, Metolachlor-S, Naproanilid, Napropamid, Pethoxamid, Piperophos, Pretilachlor, Propachlor, Propisochlor, Pyroxasulfon (KIH-485) and Thenylchlor;

Compounds of the formula 2:

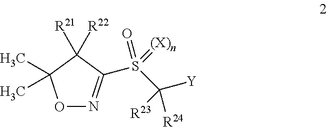

Particularly preferred Compounds of the formula 2 are:

3-[5-(2,2-Difluor-ethoxy)-1-methyl-3-trifluormethyl-1H-pyrazol-4-ylmethansulfonyl]-4-fluor-5,5-dimethyl-4,5-dihydro-isoxazol (2-1); 3-{[5-(2,2-Difluor-ethoxy)-1-methyl-3-trifluormethyl-1H-pyrazol-4-yl]-fluor-methansulfonyl}-5,5-dimethyl-4,5-dihydro-isoxazol (2-2); 4-(4-Fluor-5,5-dimethyl-4,5-dihydro-isoxazol-3-sulfonylmethyl)-2-methyl-5-trifluormethyl-2H-[1,2,3]triazol (2-3); 4-[(5,5-Dimethyl-4,5-dihydro-isoxazol-3-sulfonyl)-fluor-methyl]-2-methyl-5-trifluormethyl-2H-[1,2,3]triazol (2-4); 4-(5,5-Dimethyl-4,5-dihydro-isoxazol-3-sulfonylmethyl)-2-methyl-5-trifluormethyl-2H-[1,2,3]triazol (2-5); 3-{[5-(2,2-Difluor-ethoxy)-1-methyl-3-trifluormethyl-1H-pyrazol-4-yl]-difluor-methansulfonyl}-5,5-dimethyl-4,5-dihydro-isoxazol (2-6); 4-[(5,5-Dimethyl-4,5-dihydro-isoxazol-3-sulfonyl)-difluor-methyl]-2-methyl-5-trifluormethyl-2H-[1,2,3]triazol (2-7); 3-{[5-(2,2-Difluor-ethoxy)-1-methyl-3-trifluormethyl-1H-pyrazol-4-yl]-difluor-methansulfonyl}-4-fluor-5,5-dimethyl-4,5-dihydro-isoxazol (2-8); 4-[Difluor-(4-fluor-5,5-dimethyl-4,5-dihydro-isoxazol-3-sulfonyl)-methyl]-2-methyl-5-trifluormethyl-2H-[1,2,3]triazol (2-9);
k) from the group of Cellulose-Biosynthesis-Inhibitors:

### Chlorthiamid, Dichlobenil, Flupoxam and Isoxaben;

l) from the group of Uncoupling-Herbicides:
Dinoseb, Dinoterb and DNOC and its salts;
m) from the group of Auxin-Herbicides:
2,4-D and its salts and esters, 2,4-DB and its salts and esters, Aminopyralid and its salts wie Aminopyralid-tris(2-hydroxypropyl)ammonium and its esters, Benazolin, Benazolinethyl, Chloramben and its salts and esters, Clomeprop, Clopyralid and its salts and esters, Dicamba and its salts and esters, Dichlorpropand its salts and esters, Dichlorprop-P and its salts and esters, Fluoroxypyr, Fluoroxypyr-butomethyl, Fluoroxypyr-meptyl, MCPA and its salts and esters, MCPA-thioethyl, MCPB and its salts and esters, Mecoprop and its salts and esters, Mecoprop-P and its salts and esters, Picloram and its salts and esters, Quinclorac, Quinmerac, TBA (2,3,6) and its salts and esters, Triclopyr and its salts and esters, and 5,6-Dichlor-2-cyclopropyl-4-pyrimidincarbonic acid (H-9; CAS 858956-08-8) and its salts and esters;
n) from the group of Auxin-Transport-Inhibitors: Diflufenzopyr, Diflufenzopyr-natrium, Naptalam and Naptalam-natrium;
o) from the group of other Herbicides: Bromobutid, Chlorflurenol, Chlorflurenol-methyl, Cinmethylin, Cumyluron, Dalapon, Dazomet, Difenzoquat, Difenzoquat-metilsulfate, Dimethipin, DSMA, Dymron, Endothal and its salts, Etobenzanid, Flamprop, Flamprop-isopropyl, Flamprop-methyl Flamprop-M-isopropyl, Flamprop-M-methyl, Flurenol, Flurenol-butyl, Flurprimidol, Fosamin, Fosamine-ammonium, Indanofan, Maleinic acid-hydrazid, Mefluidid, Metam, Methylazid, Methylbromid, Methyl-dymron, Methyljodid. MSMA, oleic acid, Oxaziclomefon, Pelargonic acid, Pyributicarb, Quinoclamin, Triaziflam, Tridiphan and 6-Chlor-3-(2-cyclopropyl-6-methylphenoxy)-4-pyridazinol (H-10; CAS 499223-49-3) and its salts and esters.

Examples for preferred Safeners C are Benoxacor, Cloquintocet, Cyometrinil, Cyprosulfamid, Dichlormid, Dicyclonon, Dietholate, Fenchlorazol, Fenclorim, Flurazol, Fluxofenim, Furilazol, Isoxadifen, Mefenpyr, Mephenat, Naphthalic acid anhydrid, Oxabetrinil, 4-(Dichloracetyl)-1-oxa-4-azaspiro[4.5]decan (H-11; MON4660, CAS 71526-07-3) and 2,2,5-Trimethyl-3-(dichloracetyl)-1,3-oxazolidin (H-12; R-29148, CAS 52836-31-4).

The compounds of groups a) to o) and the Safeners C are known Herbicides and Safeners, see e.g. The Compendium of Pesticide Common Names (http://www.alanwood.net/pesticides/); B. Hock, C. Fedtke, R. R. Schmidt, Herbicides, Georg Thieme Verlag, Stuttgart 1995. Other herbicidal effectors are known from WO 96/26202, WO 97/41116, WO 97/41117, WO 97/41118, WO 01/83459 and WO 2008/074991 as well as from W. Krämer et al. (ed.) “Modern Crop Protection Compounds”, Vol. 1, Wiley VCH, 2007 and the literature cited therein

Preferred examples of herbicides include, but are not limited to, saflufenacil, benzoxazinone-derivative herbicide (as disclosed in WO2010/145992), flumioxazin, butafenacil, acifluorfen, lactofen, bifenox, diuron, sulfentrazone.

Particularly preferred herbicides useful for the present invention, are coumarone-derivative herbicides disclosed in WO2010/049270, WO2010/049269, WO2010/139657, WO2010/139658, EP2325170, WO2011/057989, WO2011/058036, WO2011/117195, WO2011/117211, WO2011/117210, WO2011/117273, WO2011/117151, WO2011/117152, WO2010/029311, WO2009/090401, WO2009/090402, WO2008/071918, WO2008/009908, WO 2012/084755, WO 2012/085265, PCT/EP2012/060846, PCT/EP2012/060600, WO2010130970.

Further particularly preferred herbicides useful for the present invention, comprise azines of formula (I)

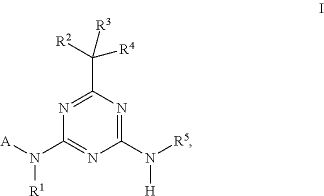

wherein
A is phenyl, which is substituted by two to five substituents selected from the group consisting of halogen, CN, NO₂, C₁-C₆-alkyl, C₁-C₆-haloalkyl, OH, C₁-C₆-alkoxy, C₁-C₆-alkylthio, (C₁-C₆-alkyl)sulfinyl, (C₁-C₆-alkyl)sulfonyl, amino, (C₁-C₆-alkyl)amino, di(C₁-C₆-alkyl)amino, (C₁-C₆-alkyl)carbonyl, (C₁-C₆-alkoxy)carbonyl;
R¹H, CN, C₁-C₆-alkyl, C₁-C₆-alkoxy-C₁-C₆-alkyl, C₁-C₆-alkoxy, (C₁-C₆-alkyl)carbonyl, (C₁-C₆-alkoxy)carbonyl, (C₁-C₆-alkyl)sulfonyl or phenylsulfonyl,
   wherein the phenyl is unsubstituted or substituted by one to five substituents selected from the group consisting of halogen, CN, NO₂, C₁-C₆-alkyl, C₁-C₆-haloalkyl and C₁-C₆-alkoxy;
R²H, halogen, CN, C₁-C₆-alkyl, C₁-C₆-haloalkyl, C₂-C₆-alkenyl, C₃-C₆-alkynyl, C₃-C₆-cycloalkyl, C₃-C₆-cycloalkenyl, OH, C₁-C₆-alkoxy or C₁-C₆-alkoxy-C₁-C₆-alkyl;
R³H, halogen, CN, C₁-C₆-alkyl, C₁-C₆-haloalkyl or C₁-C₆-alkoxy;
R⁴H, halogen, CN, C₁-C₆-alkyl or C₁-C₆-haloalkyl; or
R³ and R⁴ together with the carbon atom to which they are attached form a moiety selected from the group consisting of carbonyl, C₂-C₆-alkenyl, C₃-C₆-cycloalkyl, C₃-C₆-cycloalkenyl and three- to six-membered heterocyclyl,
   wherein the C₃-C₆-cycloalkyl, C₃-C₆-cycloalkenyl, or three- to six-membered heterocyclyl is unsubstituted or substituted by one to three substituents selected from halogen, CN, C₁-C₆-alkyl and C₁-C₆-alkoxy; and
R⁵H, CN, C₁-C₆-alkyl, C₁-C₆-alkoxy-C₁-C₆-alkyl, C₁-C₆-alkoxy, (C₁-C₆-alkyl)carbonyl, (C₁-C₆-alkoxy)carbonyl, (C₁-C₆-alkyl)sulfonyl or phenylsulfonyl,
   wherein the phenyl is unsubstituted or substituted by one to five substituents selected from the group consisting of halogen, CN, NO₂, C₁-C₆-alkyl, C₁-C₆-haloalkyl and C₁-C₆-alkoxy;
      including their agriculturally acceptable salts or N-oxides.

Further particularly preferred herbicides useful for the present invention, comprise triazines of formula (I)

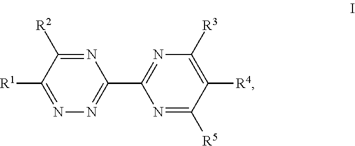

wherein
R¹ is hydrogen, C₁-C₁₀-alkyl, C₂-C₁₀-alkenyl, C₂-C₁₀-alkynyl, C₁-C₈-haloalkyl, C₂-C₈-haloalkenyl, C₂-C₈-haloalkynyl, C₁-C₈-alkoxy, C₁-C₈-alkoxy-C₁-C₈-alkyl, C₁-C₈-alkylthio, C₁-C₈-alkylthio-C₁-C₈-alkyl, hydroxy-C₁-C₁₀-alkyl, aminocarbonyl, (C₁-C₆-alkyl)aminocarbonyl, di(C₁-C₆-alkyl)aminocarbonyl, C₃-C₆-cycloalkyl, C₃-C₆-cycloalkyl-C₁-C₈-alkyl, C₃-C₆-cycloalkyl-C₁-C₈-haloalkyl, C₃-C₆-cycloalkyl-C₂-C₈-alkenyl or C₃-C₆-cycloalkyl-C₂-C₈-haloalkenyl,
   which cycloalkyls are unsubstituted or substituted by one to five substituents selected from the group consisting of halogen, C₁-C₆-alkyl, C₂-C₆-alkenyl, C₂-C₆-alkynyl, C₁-C₆-haloalkyl, C₂-C₆-haloalkenyl and C₂-C₆-haloalkynyl;
R² is halogen, CN, OH, SH, C₁-C₆-alkoxy, C₁-C₆-alkylthio, C₁-C₆-alkoxy-C₁-C₄-alkoxy, C₁-C₆-alkoxy-C₁-C₄-alkylthio, C₁-C₆-alkylthio-C₁-C₄-alkoxy, C₁-C₆-alkyltio-C₁-C₄-alkylthio, C₁-C₆-alkoxycarbonyl-C₁-C₆-alkoxy, C₁-C₆-alkoxycarbonyloxy, C₁-C₆-alkylthiocarbonyloxy, aminocarbonyloxy, (C₁-C₆-alkyl)aminocarbonyloxy, di(C₁-C₆-alkyl)aminocarbonyloxy, a 5-membered heteroaryl having 1 to 4 nitrogen atoms,
   which heteroaryl is attached to the triazine ring via a nitrogen atom, and which heteroaryl is unsubstituted or substituted by 1 to 4 substituents selected from the group consisting of halogen, CN, NO₂, OH, C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₁-C₆-alkoxy, C₁-C₆-alkylthio, C₁-C₆-alkoxycarbonyl, C₁-C₆-alkylthiocarbonyl, C₁-C₆-alkoxy-C₁-C₄-alkyl, C₁-C₆-alkylthio-C₁-C₄-alkyl, hydroxycarbonyl, thiocarboxy, C₁-C₆-alkoxycarbonyl-C₁-C₄-alkyl, C₁-C₆-alkylthiocarbonyl-C₁-C₄-alkyl, NH₂, (C₁-C₆-alkyl)amino and di(C₁-C₆-alkyl)amino,
phenoxy or phenyl-C₁-C₄-alkoxy,
   which phenyls are unsubstituted or substituted by one to five substituents selected from the group consisting of halogen, CN, NO₂, C₁-C₄-alkyl, C₁-C₄-alkoxy, C₁-C₄-haloalkyl and C₁-C₄-haloalkoxy;
R³, R⁴ and R⁵ independently of one another are hydrogen, halogen, CN, NO₂, C₁-C₆-alkyl, C₁-C₆-haloalkyl, C₁-C₆-alkoxy, C₁-C₆-haloalkoxy, C₁-C₆-alkoxy-C₁-C₆-alkyl, C₁-C₆-alkylsulfonyl, (C₁-C₆-alkyl)amino or di(C₁-C₆-alkyl)amino;
including their agriculturally acceptable salts or, provided that the triazines of formula I have a carboxyl group, their agriculturally acceptable derivatives.

Preferred are the triazines of the formula (I), wherein
R¹ is hydrogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁-C₆-alkoxy, C₁-C₆-alkylthio, aminocarbonyl, (C₁-C₆-alkyl)aminocarbonyl, di(C₁-C₆-alkyl)aminocarbonyl, C₃-C₆-cycloalkyl, C₃-C₆-cycloalkyl-C₁-C₆-alkyl, C₃-C₆-cycloalkyl-C₁-C₆-haloalkyl, C₃-C₆-cycloalkyl-C₂-C₆-alkenyl or C₃-C₆-cycloalkyl-C₂-C₆-haloalkenyl,
   which cycloalkyls are unsubstituted or substituted by one to five substituents selected from the group consisting of halogen, C₁-C₆-alkyl, C₂-C₆-alkenyl, C₂-C₆-alkynyl, C₁-C₆-haloalkyl, C₂-C₆-haloalkenyl and C₂-C₆-haloalkynyl;
R² is halogen, CN, OH, SH, C₁-C₆-alkoxy, C₁-C₆-alkylthio, C₁-C₆-alkoxy-C₁-C₄-alkoxy, C₁-C₆-alkoxycarbonyl-C₁-C₆-alkoxy, C₁-C₆-alkoxycarbonyloxy, C₁-C₆-alkylthiocarbonyloxy, aminocarbonyloxy, (C₁-C₆-alkyl)aminocarbonyloxy, di(C₁-C₆-alkyl)aminocarbonyloxy or a 5-membered heteroaryl having 1 to 4 nitrogen atoms,
   which heteroaryl is attached to the triazine ring via a nitrogen atom, and which heteroaryl is unsubstituted or substituted by 1 to 4 substituents selected from the group consisting of halogen, CN, NO₂, OH, C₁-C₆-alkyl, C₂-C₆-alkenyl, C₂-C₆-alkynyl, C₃-C₆-cycloalkyl, C₃-C₆-cycloalkenyl, C₁-C₆-haloalkyl, C₂-C₆-haloalkenyl, C₂-C₆-haloalkynyl, C₁-C₆-alkoxy, C₁-C₆-alkylthio, C₁-C₆-alkoxycarbonyl, C₁-C₆-alkylthiocarbonyl, hydroxycarbonyl, thiocarboxy, C₁-C₆-alkoxycarbonyl-C₁-C₄-alkyl, C₁-C₆-alkylthiocarbonyl-C₁-C₄-alkyl, NH₂, (C₁-C₆-alkyl)amino and di(C₁-C₆-alkyl)amino;
R³, R⁴ and R⁵ independently of one another are hydrogen, halogen, CN, NO₂, C₁-C₆-alkyl, C₁-C₆-haloalkyl, C₁-C₆-alkoxy, C₁-C₆-haloalkoxy, C₁-C₆-alkoxy-C₁-C₆-alkyl, C₁-C₆-alkylsulfonyl, (C₁-C₆-alkyl)amino or di(C₁-C₆-alkyl)amino;
   including their agriculturally acceptable salts or, provided that the triazines of formula I have a carboxyl group, their agriculturally acceptable derivatives.

Further particularly preferred herbicides useful for the present invention, comprise pyrazol amide compounds of formula (I)

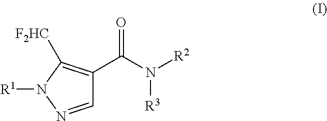

wherein
R¹ is H, C₁-C₁₀-alkyl, C₁-C₁₀-haloalkyl, C₂-C₁₀-hydroxyalkyl, C₁-C₆-alkoxy-C₁-C₆-alkyl, C₁-C₆-haloalkoxy-C₁-C₆-alkyl, C₁-C₆-alkylthio-C₁-C₆-alkyl, C₁-C₆-haloalkylthio-C₁-C₆-alkyl, C₁-C₆-alkylsulfinyl-C₁-C₆-alkyl, C₁-C₆-haloalkylsulfinyl-C₁-C₆-alkyl, C₁-C₆-alkylsulfonyl-C₁-C₆-alkyl, C₁-C₆-haloalkylsulfonyl-C₁-C₆-alkyl, C₃-C₆-cycloalkyl, 1-methylcycloprop-1-yl, 2-methylcycloprop-1-yl, 2,2-dimethylcycloprop-1-yl, 2,2,3,3-tetramethylcycloprop-1-yl, C₃-C₆-halocycloalkyl, C₃-C₆-cycloalkyl-C₁-C₆-alkyl, C₃-C₆-halocycloalkyl-C₁-C₆-alkyl, C₂-C₁₀-alkenyl, C₂-C₁₀-haloalkenyl, C₂-C₁₀-hydroxyalkenyl, C₃-C₁₀-alkadienyl, C₃-C₆-alkynyl, C₃-C₆-haloalkynyl, CH₂CN, CH(CN)₂, N,N-di-(C₁-C₆)-alkylamino-C₁-C₆-alkyl, C₁-C₆-dialkoxy-C₁-C₆-alkyl, C₁-C₆-dialkylthio-C₁-C₆-alkyl, C₁-C₃-alkoxy-C₁-C₃-alkylthio-C₁-C₆-alkyl, or a heterocyclic group selected from the formulae H1, H2 or H3

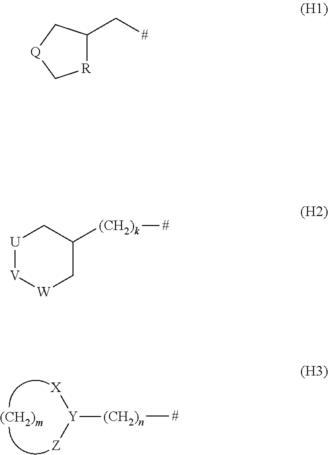

wherein
each of Q and R in the formula H1 is O or S;
U—V—W in the formula H2 is selected from the group consisting of CH₂—CH₂—O, CH₂—CH₂—NH, CH₂—CH₂—N(CH₃), CH₂—O—CH₂, CH₂—NH—CH₂, CH₂—N(CH₃)—CH₂, O—CH₂—O, O—CH₂—S, and S—CH₂—S;
k in the formula H2 is 0 or 1;
X—Y—Z in the formula H3 is selected from the group consisting of CH₂—N—CH₂, O—CH—CH₂, O—CH—O, S—CH—CH₂, S—CH—S, and O—CH—S;
m in the formula H3 is 1, 2 or 3;
n in the formula H3 is 0, 1 or 2, with the proviso that, when n is 0, X—Y—Z is not CH₂—N—CH₂;
# in each of the formulae H1, H2 or H3 denotes the bonding site to the remainder of the formula I;
R² is hydrogen, C₁-C₄-alkyl, C₃-C₆-cycloalkyl or C₁-C₄-alkoxy; and
R³ is hydrogen, C₁-C₄-alkyl, C₃-C₆-cycloalkyl or C₁-C₄-alkoxy;
   or an agriculturally acceptable salt thereof.

Further particularly preferred herbicides useful for the present invention, comprise pyrazol amide compounds of formula (II):

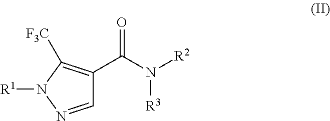

wherein
R¹ is C₅-C₁₀-alkyl, C₁-C₁₀-haloalkyl, C₂-C₁₀-hydroxyalkyl, C₁-C₆-alkoxy-C₁-C₆-alkyl, C₁-C₆-haloalkoxy-C₁-C₆-alkyl, C₁-C₆-alkylthio-C₁-C₆-alkyl, C₁-C₆-haloalkylthio-C₁-C₆-alkyl, C₁-C₆-alkylsulfinyl-C₁-C₆-alkyl, C₁-C₆-haloalkylsulfinyl-C₁-C₆-alkyl, C₁-C₆-alkylsulfonyl-C₁-C₆-alkyl, C₁-C₆-haloalkylsulfonyl-C₁-C₆-alkyl, C₃-C₄-cycloalkyl, 1-methylcycloprop-1-yl, 2-methylcycloprop-1-yl, 2,2-dimethylcycloprop-1-yl, 2,2,3,3-tetramethylcycloprop-1-yl, C₃-C₆-halocycloalkyl, C₃-C₆-cycloalkyl-C₁-C₆-alkyl, C₂-C₁₀-alkenyl, C₂-C₁₀-hydroxyalkenyl, C₃-C₁₀-alkadienyl, C₂-C₆-haloalkenyl, C₃-C₆-alkynyl, C₃-C₆-haloalkynyl, CH₂CN, CH(CN)₂, N,N-di-(C₁-C₆)-alkylamino-C₁-C₆-alkyl, C₁-C₆-dialkoxy-C₁-C₆ alkyl, C₁-C₆-dialkylthio-C₁-C₆ alkyl, C₁-C₃-alkoxy-C₁-C₃-alkylthio-C₁-C₆ alkyl, or a heterocyclic group selected from the formulae H1, H2 or H3

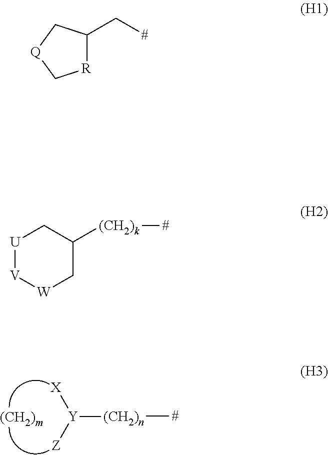

wherein
each of Q and R in the formula H1 is O or S;
U—V—W in the formula H2 is selected from the group consisting of CH₂—CH₂—O, CH₂—CH₂—NH, CH₂—CH₂—N(CH₃), CH₂—O—CH₂, CH₂—NH—CH₂, CH₂—N(CH₃)—CH₂, O—CH₂—O, O—CH₂—S, and S—CH₂—S;
k in the formula H2 is 0 or 1;
X—Y—Z in the formula H3 is selected from the group consisting of CH₂—N—CH₂, O—CH—CH₂, O—CH—O, S—CH—CH₂, S—CH—S, and O—CH—S;
m in the formula H3 is 1, 2 or 3;
n in the formula H3 is 0, 1 or 2, with the proviso that, when n is 0, X—Y—Z is not CH₂—N—CH₂;
# in each of the formulae H1, H2 or H3 denotes the bonding site to the remainder of the formula I;
R² is hydrogen, C₁-C₄-alkyl, C₃-C₆-cycloalkyl or C₁-C₄-alkoxy; and
R³ is hydrogen, C₁-C₄-alkyl, C₃-C₆-cycloalkyl or C₁-C₄-alkoxy;
   or an agriculturally acceptable salt thereof.

Unless otherwise specified, the terms “polynucleotides”, “nucleic acid” and “nucleic acid molecule” are interchangeably in the present context. Unless otherwise specified, the terms “peptide”, “polypeptide” and “protein” are interchangeably in the present context. The term “sequence” may relate to polynucleotides, nucleic acids, nucleic acid molecules, peptides, polypeptides and proteins, depending on the context in which the term “sequence” is used. The terms “gene(s)”, “polynucleotide”, “nucleic acid sequence”, “nucleotide sequence”, or “nucleic acid molecule(s)” as used herein refers to a polymeric form of nucleotides of any length, either ribonucleotides or deoxyribonucleotides. The terms “gene(s)”, “polynucleotide”, “nucleic acid sequence”, “nucleotide sequence”, or “nucleic acid molecule(s)” as used herein include known types of modifications, for example, methylation, “caps”, substitutions of one or more of the naturally occurring nucleotides with an analogue. Preferably, the DNA or RNA sequence comprises a coding sequence encoding the herein defined polypeptide. As also used herein, the terms “nucleic acid” and “nucleic acid molecule” are intended to include DNA molecules (e.g. cDNA or genomic DNA) and RNA molecules (e.g. mRNA) and analogs of the DNA or RNA generated using nucleotide analogs. The nucleic acid molecule can be single-stranded or double-stranded.

An “isolated” nucleic acid molecule is one that is substantially separated from other nucleic acid molecules, which are present in the natural source of the nucleic acid. That means other nucleic acid molecules are present in an amount less than 5% based on weight of the amount of the desired nucleic acid, preferably less than 2% by weight, more preferably less than 1% by weight, most preferably less than 0.5% by weight. Preferably, an “isolated” nucleic acid is free of some of the sequences that naturally flank the nucleic acid (i.e., sequences located at the 5′ and 3′ ends of the nucleic acid) in the genomic DNA of the organism from which the nucleic acid is derived. For example, in various embodiments, the isolated herbicide resistance and/or tolerance related protein encoding nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be free from some of the other cellular material with which it is naturally associated, or culture medium when produced by recombinant techniques, or chemical precursors or other chemicals when chemically synthesized.

A “coding sequence” is a nucleotide sequence, which is transcribed into an RNA, e.g. a regulatory RNA, such as a miRNA, a ta-siRNA, co-suppression molecule, an RNAi, a ribozyme, etc. or into a mRNA which is translated into a polypeptide when placed under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleotide sequences or genomic DNA, while introns may be present as well under certain circumstances.

As used in the present context a nucleic acid molecule may also encompass the untranslated sequence located at the 3′ and at the 5′ end of the coding gene region, for example 2000, preferably less, e.g. 500, preferably 200, especially preferable 100, nucleotides of the sequence upstream of the 5′ end of the coding region and for example 300, preferably less, e.g. 100, preferably 50, especially preferable 20, nucleotides of the sequence downstream of the 3′ end of the coding gene region.

“Polypeptide” refers to a polymer of amino acid (amino acid sequence) and does not refer to a specific length of the molecule. Thus, peptides and oligopeptides are included within the definition of polypeptide. This term does also refer to or include post-translational modifications of the polypeptide, for example, glycosylations, acetylations, phosphorylations and the like. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring. An “isolated” polynucleotide or nucleic acid molecule is separated from other polynucleotides or nucleic acid molecules, which are present in the natural source of the nucleic acid molecule. An isolated nucleic acid molecule may be a chromosomal fragment of several kb, or preferably, a molecule only comprising the coding region of the gene. Accordingly, an isolated nucleic acid molecule of the invention may comprise chromosomal regions, which are adjacent 5′ and 3′ or further adjacent chromosomal regions, but preferably comprises no such sequences which naturally flank the nucleic acid molecule sequence in the genomic or chromosomal context in the organism from which the nucleic acid molecule originates (for example sequences which are adjacent to the regions encoding the 5′- and 3′-UTRs of the nucleic acid molecule). An “isolated” or “purified” polypeptide or biologically active portion thereof is free of some of the cellular material when produced by recombinant DNA techniques, or chemical precursors or other chemicals when chemically synthesized. The language “substantially free of cellular material” includes preparations of a protein in which the polypeptide is separated from some of the cellular components of the cells in which it is naturally or recombinantly produced.

The terms “comprise” or “comprising” and grammatical variations thereof when used in this specification are to be taken to specify the presence of stated features, integers, steps or components or groups thereof, but not to preclude the presence or addition of one or more other features, integers, steps, components or groups thereof.

In accordance with the invention, a protein or polypeptide has the “activity of a CytP450 protein if its de novo activity, or its increased expression directly or indirectly leads to and confers increased herbicide tolerance or resistance, as compared to a corresponding, e.g. non-transformed, wild type plant and the protein has the above mentioned activity of a CytP450.

Throughout the specification the activity or preferably the biological activity of such a protein or polypeptide or an nucleic acid molecule or sequence encoding such protein or polypeptide is identical or similar if it still has the biological or enzymatic activity of a protein comprising the sequence of SEQ ID NO: 2, 4, 6, 8, 27, or 45, or a homolog thereof, or which has 10% or more of the original enzymatic activity, preferably 20%, 30%, 40%, 50%, particularly preferably 60%, 70%, 80% most particularly preferably 90%, 95%, 98%, 99% or more in comparison to a protein comprising the sequence of SEQ ID NO: 2, 4, 6, 8, 27, or 45, or a homolog thereof.

In another embodiment the biological or enzymatic activity of a protein comprising the sequence of SEQ ID NO: 2, 4, 6, 8, 27, or 45, or a homolog thereof, has 100% or more of the original enzymatic activity, preferably 110%, 120%, 130%, 150%, particularly preferably 150%, 200%, 300% or more in comparison to a protein comprising the sequence of SEQ ID NO: 2, 4, 6, 8, 27, or 45, or a homolog thereof.

The terms “increased”, “raised”, “extended”, “enhanced”, “improved” or “amplified” relate to a corresponding change of a property in a plant, an organism, a part of an organism such as a tissue, seed, root, leave, flower etc. or in a cell and are interchangeable. Preferably, the overall activity in the volume is increased or enhanced in cases if the increase or enhancement is related to the increase or enhancement of an activity of a gene product, independent whether the amount of gene product or the specific activity of the gene product or both is increased or enhanced or whether the amount, stability or translation efficacy of the nucleic acid sequence or gene encoding for the gene product is increased or enhanced.

The terms “increase” include the change of said property in only parts of the subject of the present invention, for example, the modification can be found in compartment of a cell, like a organelle, or in a part of a plant, like tissue, seed, root, leave, flower etc. but is not detectable if the overall subject, i.e. complete cell or plant, is tested. Accordingly, the term “increase” means that the specific activity of an enzyme as well as the amount of a compound or metabolite, e.g. of a polypeptide, a nucleic acid molecule of the invention or an encoding mRNA or DNA, can be increased in a volume. The term “increase” includes, that a compound or an activity, especially an activity, is introduced into a cell, the cytoplasm or a sub-cellular compartment or organelle de novo or that the compound or the activity, especially an activity, has not been detected before, in other words it is “generated”. Accordingly, in the following, the term “increasing” also comprises the term “generating” or “stimulating”. The increased activity manifests itself in increased herbicide tolerance or resistance, as compared to a corresponding, e.g. non-transformed, wild type plant cell, plant or part thereof.

Under “change of a property” it is understood that the activity, expression level or amount of a gene product or the metabolite content is changed in a specific volume relative to a corresponding volume of a control, reference or wild type, including the de novo creation of the activity or expression.

“Amount of protein or mRNA” is understood as meaning the molecule number of polypeptides or mRNA molecules in an organism, especially a plant, a tissue, a cell or a cell compartment. “Increase” in the amount of a protein means the quantitative increase of the molecule number of said protein in an organism, especially a plant, a tissue, a cell or a cell compartment such as an organelle like a plastid or mitochondria or part thereof—for example by one of the methods described herein below—in comparison to a wild type, control or reference.

The increase in molecule number amounts preferably to 1% or more, preferably to 10% or more, more preferably to 30% or more, especially preferably to 50%, 70% or more, very especially preferably to 100%, most preferably to 500% or more. However, a de novo expression is also regarded as subject of the present invention.

The terms “wild type”, “control” or “reference” are exchangeable and can be a cell or a part of organisms such as an organelle like a chloroplast or a tissue, or an organism, in particular a plant, which was not modified or treated according to the herein described process according to the invention. Accordingly, the cell or a part of organisms such as an organelle like a chloroplast or a tissue, or an organism, in particular a plant used as wild type, control or reference corresponds to the cell, organism, plant or part thereof as much as possible and is in any other property but in the result of the process of the invention as identical to the subject matter of the invention as possible. Thus, the wild type, control or reference is treated identically or as identical as possible, saying that only conditions or properties might be different which do not influence the quality of the tested property.

Preferably, any comparison is carried out under analogous conditions. The term “analogous conditions” means that all conditions such as, for example, culture or growing conditions, soil, nutrient, water content of the soil, temperature, humidity or surrounding air or soil, assay conditions (such as buffer composition, temperature, substrates, pathogen strain, concentrations and the like) are kept identical between the experiments to be compared.

The “reference”, “control”, or “wild type” is preferably a subject, e.g. an organelle, a cell, a tissue, an organism, in particular a plant, which was not modified or treated according to the herein described process of the invention and is in any other property as similar to the subject matter of the invention as possible. The reference, control or wild type is in its genome, transcriptome, proteome or metabolome as similar as possible to the subject of the present invention. Preferably, the term “reference-” “control-” or “wild type-”-organelle, -cell, -tissue or -organism, in particular plant, relates to an organelle, cell, tissue or organism, in particular plant, which is nearly genetically identical to the organelle, cell, tissue or organism, in particular plant, of the present invention or a part thereof preferably 90% or more, e.g. 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is a subject, e.g. an organelle, a cell, a tissue, an organism, in particular a plant, which is genetically identical to the organism, in particular plant, cell, a tissue or organelle used according to the process of the invention except that the responsible or activity conferring nucleic acid molecules or the gene product encoded by them are amended, manipulated, exchanged or introduced according to the inventive process. In case, a control, reference or wild type differing from the subject of the present invention only by not being subject of the process of the invention can not be provided, a control, reference or wild type can be an organism in which the cause for the modulation of an activity conferring the enhanced tolerance or resistance to herbicides as compared to a corresponding, e.g. non-transformed, wild type plant cell, plant or part thereof or expression of the nucleic acid molecule of the invention as described herein has been switched back or off, e.g. by knocking out the expression of responsible gene product, e.g. by antisense or RNAi or miRNA inhibition, by inactivation of an activator or agonist, by activation of an inhibitor or antagonist, by inhibition through adding inhibitory antibodies, by adding active compounds as e.g. hormones, by introducing negative dominant mutants, etc. A gene production can for example be knocked out by introducing inactivating point mutations, which lead to an enzymatic activity inhibition or a destabilization or an inhibition of the ability to bind to cofactors etc. Accordingly, preferred reference subject is the starting subject of the present process of the invention. Preferably, the reference and the subject matter of the invention are compared after standardization and normalization, e.g. to the amount of total RNA, DNA, or protein or activity or expression of reference genes, like housekeeping genes, such as ubiquitin, actin or ribosomal proteins.

The term “expression” refers to the transcription and/or translation of a codogenic gene segment or gene. As a rule, the resulting product is an mRNA or a protein.

The increase or modulation according to this invention can be constitutive, e.g. due to a stable permanent transgenic expression or to a stable mutation in the corresponding endogenous gene encoding the nucleic acid molecule of the invention or to a modulation of the expression or of the behavior of a gene conferring the expression of the polypeptide of the invention, or transient, e.g. due to an transient transformation or temporary addition of a modulator such as a agonist or antagonist or inducible, e.g. after transformation with a inducible construct carrying the nucleic acid molecule of the invention under control of a inducible promoter and adding the inducer, e.g. tetracycline or as described herein below.

Less influence on the regulation of a gene or its gene product is understood as meaning a reduced regulation of the enzymatic activity leading to an increased specific or cellular activity of the gene or its product. An increase of the enzymatic activity is understood as meaning an enzymatic activity, which is increased by 10% or more, advantageously 20%, 30% or 40% or more, especially advantageously by 50%, 60% or 70% or more in comparison with the starting organism. This leads to increased herbicide tolerance or resistance, as compared to a corresponding, e.g. non-transformed, wild type plant or part thereof.

The increase in activity of the polypeptide amounts in a cell, a tissue, an organelle, an organ or an organism, preferably a plant, or a part thereof preferably to 5% or more, preferably to 20% or to 50%, especially preferably to 70%, 80%, 90% or more, very especially preferably are to 100%, 150% or 200%, most preferably are to 250% or more in comparison to the control, reference or wild type. In one embodiment the term increase means the increase in amount in relation to the weight of the organism or part thereof (w/w).

By “vectors” is meant with the exception of plasmids all other vectors known to those skilled in the art such as by way of example phages, viruses such as SV40, CMV, baculovirus, adenovirus, transposons, IS elements, phasmids, phagemids, cosmids, linear or circular DNA. These vectors can be replicated autonomously in the host organism or be chromosomally replicated, chromosomal replication being preferred. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g. bacterial vectors having a bacterial origin of replication and episomal mammalian vectors). Other vectors (e.g. non-episomal mammalian vectors) are integrated into the genome of a host cell or a organelle upon introduction into the host cell, and thereby are replicated along with the host or organelle genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses, and adeno-associated viruses), which serve equivalent functions.

As used herein, “operatively linked” is intended to mean that the nucleotide sequence of interest is linked to the regulatory sequence(s) in a manner which allows for expression of the nucleotide sequence (e.g. in an in vitro transcription/translation system or in a host cell when the vector is introduced into the host cell). The term “regulatory sequence” is intended to include promoters, enhancers, and other expression control elements (e.g. polyadenylation signals). Such regulatory sequences are described, for example, in Goeddel, Gene Expression Technology: Methods in Enzymology 185, Academic Press, San Diego, Calif. (1990), and Gruber and Crosby, in: Methods in Plant Molecular Biology and Biotechnology, eds. Glick and Thompson, Chapter 7, 89-108, CRC Press; Boca Raton, Fla., including the references therein. Regulatory sequences include those that direct constitutive expression of a nucleotide sequence in many types of host cells and those that direct expression of the nucleotide sequence only in certain host cells or under certain conditions.

“Transformation” is defined herein as a process for introducing heterologous DNA into a plant cell, plant tissue, or plant. It may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time. Transformed plant cells, plant tissue, or plants are understood to encompass not only the end product of a transformation process, but also transgenic progeny thereof.

The terms “transformed,” “transgenic,” and “recombinant” refer to a host organism such as a bacterium or a plant into which a heterologous nucleic acid molecule has been introduced. The nucleic acid molecule can be stably integrated into the genome of the host or the nucleic acid molecule can also be present as an extra-chromosomal molecule. Such an extra-chromosomal molecule can be auto-replicating. Transformed cells, tissues, or plants are understood to encompass not only the end product of a transformation process, but also transgenic progeny thereof. A “non-transformed”, “non-transgenic” or “nonrecombinant” host refers to a wild-type organism, e.g. a bacterium or plant, which does not contain the heterologous nucleic acid molecule.

The terms “host organism”, “host cell”, “recombinant (host) organism” and “transgenic (host) cell” are used here interchangeably. Of course these terms relate not only to the particular host organism or the particular target cell but also to the descendants or potential descendants of these organisms or cells. Since, due to mutation or environmental effects certain modifications may arise in successive generations, these descendants need not necessarily be identical with the parental cell but nevertheless are still encompassed by the term as used here.

For the purposes of the invention “transgenic” or “recombinant” means with regard for example to a nucleic acid sequence, an expression cassette (=gene construct, nucleic acid construct) or a vector containing the nucleic acid sequence according to the invention or an organism transformed by said nucleic acid sequences, expression cassette or vector according to the invention all those constructions produced by genetic engineering methods in which either
(a) the nucleic acid sequence comprising the sequence of SEQ ID NO: 1, 3, 5, 7, 26, or 44, or a homolog thereof, or its derivatives or parts thereof; or
(b) a genetic control sequence functionally linked to the nucleic acid sequence described under (a), for example a 3′- and/or 5′-genetic control sequence such as a promoter or terminator, or
(c) (a) and (b);
   are not found in their natural, genetic environment or have been modified by genetic engineering methods, wherein the modification may by way of example be a substitution, addition, deletion, inversion or insertion of one or more nucleotide residues.

“Natural genetic environment” means the natural genomic or chromosomal locus in the organism of origin or inside the host organism or presence in a genomic library. In the case of a genomic library the natural genetic environment of the nucleic acid sequence is preferably retained at least in part. The environment borders the nucleic acid sequence at least on one side and has a sequence length of at least 50 bp, preferably at least 500 bp, particularly preferably at least 1,000 bp, most particularly preferably at least 5,000 bp. A naturally occurring expression cassette—for example the naturally occurring combination of the natural promoter of the nucleic acid sequence according to the invention with the corresponding gene—turns into a transgenic expression cassette when the latter is modified by unnatural, synthetic (“artificial”) methods such as by way of example a mutagenation. Appropriate methods are described by way of example in U.S. Pat. No. 5,565,350 or WO 00/15815.

The term “transgenic plants” used in accordance with the invention also refers to the progeny of a transgenic plant, for example the T₁, T₂, T₃ and subsequent plant generations or the BC₁, BC₂, BC₃ and subsequent plant generations. Thus, the transgenic plants according to the invention can be raised and selfed or crossed with other individuals in order to obtain further transgenic plants according to the invention. Transgenic plants may also be obtained by propagating transgenic plant cells vegetatively. The present invention also relates to transgenic plant material, which can be derived from a transgenic plant population according to the invention. Such material includes plant cells and certain tissues, organs and parts of plants in all their manifestations, such as seeds, leaves, anthers, fibers, tubers, roots, root hairs, stems, embryo, calli, cotelydons, petioles, harvested material, plant tissue, reproductive tissue and cell cultures, which are derived from the actual transgenic plant and/or can be used for bringing about the transgenic plant. Any transformed plant obtained according to the invention can be used in a conventional breeding scheme or in in vitro plant propagation to produce more transformed plants with the same characteristics and/or can be used to introduce the same characteristic in other varieties of the same or related species. Such plants are also part of the invention. Seeds obtained from the transformed plants genetically also contain the same characteristic and are part of the invention. As mentioned before, the present invention is in principle applicable to any plant and crop that can be transformed with any of the transformation method known to those skilled in the art.

The term “homology” means that the respective nucleic acid molecules or encoded proteins are functionally and/or structurally equivalent. The nucleic acid molecules that are homologous to the nucleic acid molecules described above and that are derivatives of said nucleic acid molecules are, for example, variations of said nucleic acid molecules which represent modifications having the same biological function, in particular encoding proteins with the same or substantially the same biological function. They may be naturally occurring variations, such as sequences from other plant varieties or species, or mutations. These mutations may occur naturally or may be obtained by mutagenesis techniques. The allelic variations may be naturally occurring allelic variants as well as synthetically produced or genetically engineered variants. Structurally equivalents can, for example, be identified by testing the binding of said polypeptide to antibodies or computer based predictions. Structurally equivalent have the similar immunological characteristic, e.g. comprise similar epitopes.

As used herein, the terms “gene” and “recombinant gene” refer to nucleic acid molecules comprising an open reading frame encoding the polypeptide of the invention or comprising the nucleic acid molecule of the invention or encoding the polypeptide used in the process of the present invention, preferably from a crop plant or from a microorganism useful for the method of the invention. Such natural variations can typically result in 1 to 5% variance in the nucleotide sequence of the gene. Any and all such nucleotide variations and resulting amino acid polymorphisms in genes encoding a polypeptide of the invention or comprising a the nucleic acid molecule of the invention that are the result of natural variation and that do not alter the functional activity as described are intended to be within the scope of the invention.

### Specific Embodiments

Accordingly, this invention provides measures and methods to produce plants with increased herbicide tolerance or resistance.

Accordingly, the present invention provides transgenic plants showing increased tolerance or resistance to one or more herbicides as compared to the corresponding origin or the wild type plant and methods for producing such transgenic plants with increased herbicide tolerance or resistance. One or more enhanced herbicide tolerance-related phenotypes are increased in accordance with the invention by increasing or generating the activity of an Alopecurus CytP450 enzyme.

The nucleic acid molecule of the present invention or used in accordance with the present invention, encodes a protein conferring an activity of an Alopecurus CytP450 enzyme.

Accordingly, in one embodiment, the present invention relates to a nucleic acid molecule that encodes a polypeptide with an herbicide tolerance or resistance-increasing activity which is encoded by a nucleic acid sequence comprising the sequence of SEQ ID NO: 1, 3, 5, 7, 26, or 44, or a homolog thereof, and/or which is a protein comprising or consisting of a polypeptide comprising the sequence of SEQ ID NO: 2, 4, 6, 8, 27, or 45, or a homolog thereof.

The increase or generation of said “activity” is for example conferred by the increase of activity or of amount in a cell or a part thereof of one or more expression products of said nucleic acid molecule, e.g. proteins, or by de novo expression, i.e. by the generation of said “activity” in the plant.

In one embodiment, said herbicide tolerance or resistance-increasing activity is increased by increasing the amount and/or the specific activity of a CytP450 protein comprising the sequence of SEQ ID NO: 2, 4, 6, 8, 27, or 45, or a homolog thereof.

Accordingly, in one embodiment, an increased herbicide tolerance or resistance as compared to a correspondingly non-modified, e.g. a non-transformed, wild type plant is conferred according to method of the invention, by increasing or generating the activity of a polypeptide comprising the sequence of SEQ ID NO: 2, 4, 6, 8, 27, or 45, or a homolog thereof, or encoded by the nucleic acid molecule (or gene) the sequence of SEQ ID NO: 1, 3, 5, 7, 26, or 44, or a homolog of said nucleic acid molecule or polypeptide.

Thus, in one embodiment, the present invention provides a method for producing a plant showing increased or improved herbicide resistance or tolerance as compared to the corresponding origin or wild type plant, by increasing or generating the activity of an Alopecurus CytP450 enzyme, e.g. which is conferred by one or more polynucleotide(s) comprising the sequence of SEQ ID NO: 1, 3, 5, 7, 26, or 44, or a homolog thereof, or by one or more protein(s), each comprising a polypeptide encoded by one or more nucleic acid sequence(s) comprising the sequence of SEQ ID NO: 1, 3, 5, 7, 26, or 44, or a homolog thereof, or by one or more protein(s) each comprising a polypeptide comprising the sequence of SEQ ID NO: 2, 4, 6, 8, 27, or 45, or a homolog thereof, and (b) optionally, growing the plant cell, plant or part thereof under conditions which permit the development of the plant cell, the plant or the part thereof, and (c) regenerating a plant with increased herbicide tolerance or resistance, as compared to a corresponding, e.g. non-transformed, wild type plant or a part thereof.

Accordingly, in one further embodiment, the said method for producing a plant or a part thereof for the regeneration of said plant, the plant showing an increased herbicide tolerance or resistance, said method comprises (i) growing the plant or part thereof together with a, e.g. non-transformed, wild type plant under conditions of herbicide treatment; and (ii) selecting a plant with increased herbicide tolerance or resistance as compared to a corresponding, e.g. non-transformed, wild type plant, for example after the, e.g. non-transformed, wild type plant shows visual symptoms of deficiency and/or death.

Further, the present invention relates to a method for producing a plant with increased herbicide tolerance or resistance as compared to a corresponding origin or wild type plant, e.g. a transgenic plant, which comprises: (a) increasing or generating, in a plant cell nucleus, a plant cell, a plant or a part thereof, the activity of an Alopecurus CytP450 polypeptide of the present invention, e.g. by the methods mentioned herein; and (b) cultivating or growing the plant cell, the plant or the part thereof under conditions which permit the development of the plant cell, the plant or the part thereof; and (c) recovering a plant from said plant cell nucleus, said plant cell, or said plant part, which shows increased herbicide tolerance or resistance as compared to a corresponding, e.g. non-transformed, origin or wild type plant; and (d) optionally, selecting the plant or a part thereof, showing increased herbicide tolerance or resistance, as compared to a corresponding, e.g. non-transformed, wild type plant cell, e.g. which shows visual symptoms of deficiency and/or death.

Furthermore, the present invention also relates to a method for the identification of a plant with an increased herbicide tolerance or resistance comprising screening a population of one or more plant cell nuclei, plant cells, plant tissues or plants or parts thereof for said “activity”, comparing the level of activity with the activity level in a reference; identifying one or more plant cell nuclei, plant cells, plant tissues or plants or parts thereof with the activity increased compared to the reference, optionally producing a plant from the identified plant cell nuclei, cell or tissue.

In one further embodiment, the present invention also relates to a method for the identification of a plant with an increased herbicide tolerance or resistance comprising screening a population of one or more plant cell nuclei, plant cells, plant tissues or plants or parts thereof for the expression level of an nucleic acid coding for an polypeptide conferring said activity, comparing the level of expression with a reference; identifying one or more plant cell nuclei, plant cells, plant tissues or plants or parts thereof with the expression level increased compared to the reference, optionally producing a plant from the identified plant cell nuclei, cell or tissue.

Accordingly, in a preferred embodiment, the present invention provides a method for producing a transgenic cell for the regeneration or production of a plant with increased herbicide tolerance or resistance, as compared to a corresponding, e.g. non-transformed, wild type cell by increasing or generating the activity of an Alopecurus CytP450 polypeptide of the present invention. The cell can be for example a host cell, e.g. a transgenic host cell. A host cell can be for example a microorganism, e.g. derived from fungi or bacteria, or a plant cell particular useful for transformation.

Thus, the present invention fulfills the need to identify new, unique genes capable of conferring increased herbicide tolerance or resistance to plants, upon expression or overexpression of exogenous genes. Accordingly, the present invention provides novel CytP450 enzymes comprising the sequence of SEQ ID NO: 2, 4, 6, 8, 27, or 45, or a homolog thereof.

In one embodiment the increase in activity of the polypeptide amounts in an organelle such as a plastid. In another embodiment the increase in activity of the polypeptide amounts in the cytoplasm.

The specific activity of a polypeptide encoded by a nucleic acid molecule of the present invention or of the polypeptide of the present invention can be tested as described in the examples. In particular, the expression of a protein in question in a cell, e.g. a plant cell in comparison to a control is an easy test and can be performed as described in the state of the art.

Accordingly, in one embodiment, the process of the present invention for producing a plant with increased herbicide tolerance or resistance comprises increasing or generating the activity of a gene product conferring the activity of a CytP450 enzyme from Alopecurus or its functional equivalent or its homolog, e.g. the increase of

(a) a gene product of a gene comprising the nucleic acid molecule comprising the sequence of SEQ ID NO: 1, 3, 5, 7, 26, or 44, or a functional equivalent or a homologue thereof; or
(b) a polypeptide comprising a polypeptide, a consensus sequence or a polypeptide motif comprising the sequence of SEQ ID NO: 2, 4, 6, 8, 27, or 45 or a functional equivalent or a homologue thereof, preferably a homologue or functional equivalent comprising the sequence of SEQ ID NO: 9, 10, 11, 12, 13, 14, 15, 16, 17, 18, 19, 20, 21, 22, 23, 24, 25, 28, 29, 30, 31, 32, 33, 34, 35, 36, 37, 38, 39, 40, 41, 42, 43, 46, 47, 48, 49, 50, 51, 52, 53, 54, 55, 56, 57, 58, 59, 60, 61, 62, 63, 64, 65, 66, 67, 68, or 69.

Accordingly, an activity of a CytP450 polypeptide from Alopecurus is increased in one or more specific compartment(s) or organelle(s) of a cell or plant and confers said increased herbicide tolerance or resistance. For example, said activity can be increased in plastids or mitochondria of a plant cell, thus conferring increase of herbicide tolerance or resistance in a corresponding plant.

In one embodiment, an activity conferred by an expression of a gene described herein or its expression product; i.e. by a CytP450 polypeptide of the present invention is increased or generated in the plastid.

In one embodiment, an activity conferred by the expression of a gene described herein or its expression product; i.e. by a CytP450 polypeptide of the present invention is increased or generated in the mitochondria.

In one embodiment, an activity conferred by the expression of a gene described herein or its expression product; i.e. by a CytP450 polypeptide of the present invention is increased or generated in the cytoplasm.

In one embodiment, an activity conferred by the expression of a gene described herein or its expression product; i.e. by a CytP450 polypeptide of the present invention is increased or generated in the endoplasmic reticulum.

As the terms “cytoplasmic” and “non-targeted” shall not exclude a targeted localisation to any cell compartment for the products of the inventive nucleic acid sequences by their naturally occurring sequence properties within the background of the transgenic organism, in one embodiment, an activity as disclosed herein as being conferred by a polypeptide shown in SEQ ID NO: 2, 4, 6, 8, 27, or 45, or a homolog thereof is increase or generated non-targeted. For the purposes of the description of the present invention, the term “cytoplasmic” shall indicate, that the nucleic acid of the invention is expressed without the addition of a non-natural transit peptide encoding sequence. A non-natural transient peptide encoding sequence is a sequence which is not a natural part of a nucleic acid of the invention but is rather added by molecular manipulation steps which are well-known to the person skilled in the art. Therefore the term “cytoplasmic” shall not exclude a targeted localisation to any cell compartment for the products of the inventive nucleic acid sequences by their naturally occurring sequence properties.

In another embodiment the present invention is related to a method for producing a, e.g. transgenic, plant with increased herbicide tolerance or resistance, or a part thereof, as compared to a corresponding, e.g. non-transformed, wild type plant, which comprises
(a1) increasing or generating the activity of an Alopecurus CytP450 polypeptide, e.g. the activity of said gene or the gene product gene, in an organelle of a plant cell, or
(a2) increasing or generating the activity of a protein comprising the sequence of SEQ ID NO: 2, 4, 6, 8, 27, or 45, or a homolog thereof or as encoded by the nucleic acid sequences comprising the sequence of SEQ ID NO: 1, 3, 5, 7, 26, or 44, or a homolog thereof, and which is joined to a nucleic acid sequence encoding a transit peptide in the plant cell; or
(a3) increasing or generating the activity of a protein comprising the sequence of SEQ ID NO: 2, 4, 6, 8, 27, or 45, or a homolog thereof or as encoded by the nucleic acid sequences comprising the sequence of SEQ ID NO: 1, 3, 5, 7, 26, or 44, or a homolog thereof, and which is joined to a nucleic acid sequence encoding an organelle localization sequence, especially a chloroplast localization sequence, in a plant cell,
(a4) increasing or generating the activity of a protein comprising the sequence of SEQ ID NO: 2, 4, 6, 8, 27, or 45, or a homolog thereof or as encoded by the nucleic acid sequences comprising the sequence of SEQ ID NO: 1, 3, 5, 7, 26, or 44, or a homolog thereof, and which is joined to a nucleic acid sequence encoding an mitochondrion localization sequence in a plant cell,
   and
(b) regenerating a plant from said plant cell;
(c) growing the plant under conditions which permit the development of a plant with increased herbicide tolerance or resistance as compared to a corresponding, e.g. non-transformed, wild type plant.

The skilled worker is able to link transit peptide nucleic acid sequences to the nucleic acid sequences comprising the sequence of SEQ ID NO: 1, 3, 5, 7, 26, or 44, or a homolog thereof.

Any transit peptide may be used in accordance with the various embodiments of the present invention. For example, specific nucleic acid sequences are encoding transit peptides are disclosed by von Heijne et al. (Plant Molecular Biology Reporter, 9 (2), 104, (1991)) or other transit peptides are disclosed by Schmidt et al. (J. Biol. Chem. 268 (36), 27447 (1993)), Della-Cioppa et al. (Plant. Physiol. 84, 965 (1987)), de Castro Silva Filho et al. (Plant Mol. Biol. 30, 769 (1996)), Zhao et al. (J. Biol. Chem. 270 (11), 6081 (1995)), Römer et al. (Biochem. Biophys. Res. Commun. 196 (3), 1414 (1993)), Keegstra et al. (Annu. Rev. Plant Physiol. Plant Mol. Biol. 40, 471 (1989)), Lubben et al. (Photosynthesis Res. 17, 173 (1988)) and Lawrence et al. (J. Biol. Chem. 272 (33), 20357 (1997)), which are hereby incorporated by reference. A general review about targeting is disclosed by Kermode Allison R. in Critical Reviews in Plant Science 15 (4), 285 (1996) under the title “Mechanisms of Intracellular Protein Transport and Targeting in Plant Cells”.

Additional nucleic acid sequences encoding a transit peptide can be isolated from any organism such as microorganisms such as algae or plants containing plastids, preferably containing chloroplasts. A “transit peptide” is an amino acid sequence, whose encoding nucleic acid sequence is translated together with the corresponding structural gene. That means the transit peptide is an integral part of the translated protein and forms an amino terminal extension of the protein. Both are translated as so called “pre-protein”. In general the transit peptide is cleaved off from the pre-protein during or just after import of the protein into the correct cell organelle such as a plastid to yield the mature protein. The transit peptide ensures correct localization of the mature protein by facilitating the transport of proteins through intracellular membranes.

For example, such transit peptides, which are beneficially used in the inventive process, are derived from the nucleic acid sequence encoding a protein selected from the group consisting of ribulose bisphosphate carboxylase/oxygenase, 5-enolpyruvyl-shikimate-3-phosphate synthase, acetolactate synthase, chloroplast ribosomal protein CS17, Cs protein, ferredoxin, plastocyanin, ribulose bisphosphate carboxylase activase, tryptophan synthase, acyl carrier protein, plastid chaperonin-60, cytochrome c₅₅₂, 22-kDA heat shock protein, 33-kDa Oxygen-evolving enhancer protein 1, ATP synthase γ subunit, ATP synthase δ subunit, chlorophyll-a/b-binding proteinII-1, Oxygen-evolving enhancer protein 2, Oxygen-evolving enhancer protein 3, photosystem I: P21, photosystem I: P28, photosystem I: P30, photosystem I: P35, photosystem I: P37, glycerol-3-phosphate acyltransferases, chlorophyll a/b binding protein, CAB2 protein, hydroxymethyl-bilane synthase, pyruvate-orthophosphate dikinase, CAB3 protein, plastid ferritin, ferritin, early light-inducible protein, glutamate-1-semialdehyde aminotransferase, protochlorophyllide reductase, starch-granule-bound amylase synthase, light-harvesting chlorophyll a/b-binding protein of photosystem II, major pollen allergen Lol p 5a, plastid ClpB ATP-dependent protease, superoxide dismutase, ferredoxin NADP oxidoreductase, 28-kDa ribonucleoprotein, 31-kDa ribonucleoprotein, 33-kDa ribonucleoprotein, acetolactate synthase, ATP synthase CF₀ subunit 1, ATP synthase CF₀ subunit 2, ATP synthase CF₀ subunit 3, ATP synthase CF₀ subunit 4, cytochrome f, ADP-glucose pyrophosphorylase, glutamine synthase, glutamine synthase 2, carbonic anhydrase, GapA protein, heat-shock-protein hsp21, phosphate translocator, plastid ClpA ATP-dependent protease, plastid ribosomal protein CL24, plastid ribosomal protein CL9, plastid ribosomal protein PsCL18, plastid ribosomal protein PsCL25, DAHP synthase, starch phosphorylase, root acyl carrier protein II, betaine-aldehyde dehydrogenase, GapB protein, glutamine synthetase 2, phosphoribulokinase, nitrite reductase, ribosomal protein L12, ribosomal protein L13, ribosomal protein L21, ribosomal protein L35, ribosomal protein L40, triose phosphate-3-phosphoglyerate-phosphate translocator, ferredoxin-dependent glutamate synthase, glyceraldehyde-3-phosphate dehydrogenase, NADP-dependent malic enzyme and NADP-malate dehydrogenase, chloroplast 30S ribosomal protein PSrp-1, and the like.

In a particularly preferred embodiment, the nucleic acid sequences of the present invention are linked to a nucleic acid encoding a so-called “signal sequence peptide”. For the purposes of the present invention, “signal sequence peptide” refers to amino acid sequences of about 15 to about 50 amino acids in length which are known in the art to be generally located at the amino terminus of proteins and which are capable of targeting said proteins to the endoplasmic reticulum. The core of the signal peptide contains a long stretch of hydrophobic amino acids that has a tendency to form a single alpha-helix. In addition, many signal peptides begin with a short positively charged stretch of amino acids, which may help to enforce proper topology of the polypeptide during translocation by what is known as the positive-inside rule. At the end of the signal peptide there is typically a stretch of amino acids that is recognized and cleaved by signal peptidase. However this cleavage site is absent from transmembrane-domains that serve as signal peptides, which are sometimes referred to as signal anchor sequences. Signal peptidase may cleave during, or after completion of, translocation to generate a free signal peptide and a mature protein. The free signal peptides are then digested by specific proteases. Those skilled in the art would readily appreciate that many signal sequence peptides are known (van Heijne, G., J. Mol. Biol. 184: 99-105 (1985)) and that these peptide sequences or analogues thereof can be easily substituted as long as they fulfill the requirements for a signal peptide as described above.

The skilled worker will recognize that various other nucleic acid sequences encoding transit or signal sequence peptides can easily isolated from plastid-localized, mitochondria-localized or endoplasmic reticulum-localized proteins, which are expressed from nuclear genes as precursors and are then targeted to plastids, mitochondria or endoplasmic reticulum. Nucleic acid sequences encoding a transit or signal sequence peptide can be isolated from organelle-targeted proteins from any organism. Preferably, the transit or signal sequence peptide is isolated from an organism selected from the group consisting of the genera Acetabularia, Arabidopsis, Brassica, Capsicum, Chlamydomonas, Cururbita, Dunaliella, Euglena, Flaveria, Glycine, Helianthus, Hordeum, Lemna, Lolium, Lycopersion, Malus, Medicago, Mesembryanthemum, Nicotiana, Oenotherea, Oryza, Petunia, Phaseolus, Physcomitrella, Pinus, Pisum, Raphanus, Silene, Sinapis, Solanum, Spinacea, Stevie, Synechococcus, Triticum and Zea. More preferably, the nucleic acid sequence encoding the transit or signal sequence peptide is isolated from an organism selected from the group consisting of the species Acetabularia mediterranea, Arabidopsis thaliana, Brassica campestris, Brassica napus, Capsicum annuum, Chlamydomonas reinhardtii, Cururbita moschata, Dunaliella saline, Dunaliella tertiolecta, Euglena gracilis, Flaveria trinervia, Glycine max, Helianthus annuus, Hordeum vulgare, Lemna gibba, Lolium perenne, Lycopersion esculentum, Malus domestica, Medicago falcate, Medicago sativa, Mesembryanthemum crystallinum, Nicotiana plumbaginifolia, Nicotiana sylvestris, Nicotiana tabacum, Oenotherea hooken, Oryza sativa, Petunia hybrida, Phaseolus vulgaris, Physcomitrella patens, Pinus tunbergii, Pisum sativum, Raphanus sativus, Silene pratensis, Sinapis alba, Solanum tuberosum, Spinacea oleracea, Stevie rebaudiana, Synechococcus, Synechocystis, Triticum aestivum and Zea mays. Alternatively, nucleic acid sequences coding for transit or signal sequence peptides may be chemically synthesized either in part or wholly according to structure of transit peptide sequences disclosed in the prior art.

Such transit or signal sequence peptides encoding sequences can be used for the construction of other expression constructs. The transit or signal sequence peptides advantageously used in the inventive process and which are part of the inventive nucleic acid sequences and proteins are typically 20 to 120 amino acids, preferably 25 to 110, 30 to 100 or 35 to 90 amino acids, more preferably 40 to 85 amino acids and most preferably 45 to 80 amino acids as for transit peptides, or about 15 to about 50 amino acids as for signal sequence peptides in length and functions post-translational to direct the protein to the plastid, preferably to the chloroplast, the mitochondrion or endoplasmic reticulum. The nucleic acid sequences encoding such transit or signal sequence peptides are localized upstream of nucleic acid sequence encoding the mature protein. For the correct molecular joining of the transit or signal sequence peptide encoding nucleic acid and the nucleic acid encoding the protein to be targeted it is sometimes necessary to introduce additional base pairs at the joining position, which forms restriction enzyme recognition sequences useful for the molecular joining of the different nucleic acid molecules. This procedure might lead to very few additional amino acids at the N-terminal of the mature imported protein, which usually and preferably do not interfere with the protein function. In any case, the additional base pairs at the joining position which forms restriction enzyme recognition sequences have to be chosen with care, in order to avoid the formation of stop codons or codons which encode amino acids with a strong influence on protein folding, like e.g. proline. It is preferred that such additional codons encode small structural flexible amino acids such as glycine or alanine.

As mentioned above the nucleic acid sequence coding for a protein comprising the sequence of SEQ ID NO: 2, 4, 6, 8, 27, or 45, or a homolog thereof, can be joined to a nucleic acid sequence encoding a transit or a signal sequence peptide. The nucleic acid sequence of the gene to be expressed and the nucleic acid sequence encoding the transit or signal sequence peptide are operably linked. Therefore the transit or signal sequence peptide is fused in frame to the nucleic acid sequence coding for a protein comprising the sequence of SEQ ID NO: 2, 4, 6, 8, 27, or 45, or a homolog thereof.

The proteins translated from said inventive nucleic acid sequences are a kind of fusion proteins that means the nucleic acid sequences encoding the transit or signal sequence peptide, are joint to a gene, e.g. the nucleic acid sequences comprising the sequence of SEQ ID NO: 1, 3, 5, 7, 26, or 44, or a homolog thereof. The person skilled in the art is able to join said sequences in a functional manner. Advantageously the transit or signal sequence peptide part is cleaved off from the protein part during the transport preferably into the endoplasmic reticulum or plastids. The skilled worker knows that other short sequences are also useful in the expression of the CytP450 genes of the present invention. Furthermore the skilled worker is aware of the fact that there is not a need for such short sequences in the expression of the genes.

Alternatively to the targeting of the gene, e.g. proteins having the sequences comprising the sequence of SEQ ID NO: 2, 4, 6, 8, 27, or 45, or a homolog thereof, the nucleic acids of the invention can directly be introduced into the plastidic genome.

By transforming the plastids the intraspecies specific transgene flow is blocked, because a lot of species such as corn, cotton and rice have a strict maternal inheritance of plastids. By placing the gene e.g. the genes comprising the sequence of SEQ ID NO: 1, 3, 5, 7, 26, or 44, or a homolog thereof, or active fragments thereof in the plastids of plants, these genes will not be present in the pollen of said plants.

In another embodiment of the invention the gene, e.g. the nucleic acid molecules comprising the sequence of SEQ ID NO: 1, 3, 5, 7, 26, or 44, or a homolog thereof, used in the inventive process are transformed into mitochondria, which are metabolic active.

For a good expression in the plastids the gene, e.g. the nucleic acid sequences comprising the sequence of SEQ ID NO: 1, 3, 5, 7, 26, or 44, or a homolog thereof, are introduced into an expression cassette using a preferably a promoter and terminator, which are active in plastids, preferably a chloroplast promoter. Examples of such promoters include the psbA promoter from the gene from spinach or pea, the rbcL promoter, and the atpB promoter from corn.

In one embodiment, the process of the present invention comprises one or more of the following steps:
(a) stabilizing a protein conferring the increased expression of a protein encoded by the nucleic acid molecule of the invention or of the polypeptide of the invention having the herein-mentioned activity of an Alopecurus CytP450 and conferring increased herbicide tolerance or resistance, as compared to a corresponding, e.g. non-transformed, wild type plant cell, plant or part thereof;
(b) stabilizing an mRNA conferring the increased expression of a polynucleotide encoding a polypeptide as mentioned in (a);
(c) increasing the specific activity of a protein conferring the increased expression of a polypeptide as mentioned in (a);
(d) generating or increasing the expression of an endogenous or artificial transcription factor mediating the expression of a protein conferring the increased expression of a polypeptide as mentioned in (a);
(e) stimulating activity of a protein conferring the increased expression of a polypeptide as mentioned in (a), by adding one or more exogenous inducing factors to the organism or parts thereof;
(f) expressing a transgenic gene encoding a protein conferring the increased expression of a polypeptide as mentioned in (a); and/or
(g) increasing the copy number of a gene conferring the increased expression of a nucleic acid molecule encoding a polypeptide as mentioned in (a);
(h) increasing the expression of the endogenous gene encoding a polypeptide as mentioned in (a) by adding positive expression or removing negative expression elements, e.g. homologous recombination can be used to either introduce positive regulatory elements like for plants the 35S enhancer into the promoter or to remove repressor elements form regulatory regions. Further gene conversion methods can be used to disrupt repressor elements or to enhance to activity of positive elements-positive elements can be randomly introduced in plants by T-DNA or transposon mutagenesis and lines can be identified in which the positive elements have been integrated near to a gene of the invention, the expression of which is thereby enhanced; and/or
(i) modulating growth conditions of the plant in such a manner, that the expression or activity of the gene encoding a polypeptide as mentioned in (a), or the protein itself is enhanced;
(j) selecting of organisms with especially high activity of a polypeptide as mentioned in (a) from natural or from mutagenized resources and breeding them into the target organisms, e.g. the elite crops.

Preferably, said mRNA is encoded by the nucleic acid molecule of the present invention and/or the protein conferring the increased expression of a protein encoded by the nucleic acid molecule of the present invention alone or linked to a transit nucleic acid sequence or transit peptide encoding nucleic acid sequence or the polypeptide having the herein mentioned activity, e.g. conferring with increased herbicide tolerance or resistance, as compared to a corresponding, e.g. non-transformed, wild type plant cell, plant or part thereof after increasing the expression or activity of the encoded polypeptide or having the activity of a polypeptide having an activity as the protein comprising the sequence of SEQ ID NO: 2, 4, 6, 8, 27, or 45, or a homolog thereof.

In general, the amount of mRNA or polypeptide in a cell or a compartment of an organism correlates with the amount of encoded protein and thus with the overall activity of the encoded protein in said volume. Said correlation is not always linear, the activity in the volume is dependent on the stability of the molecules or the presence of activating or inhibiting co-factors. The activity of the abovementioned proteins and/or polypeptides encoded by the nucleic acid molecule of the present invention can be increased in various ways. For example, the activity in an organism or in a part thereof, like a cell, is increased via increasing the gene product number, e.g. by increasing the expression rate, like introducing a stronger promoter, or by increasing the stability of the mRNA expressed, thus increasing the translation rate, and/or increasing the stability of the gene product, thus reducing the proteins decayed. Further, the activity or turnover of enzymes can be influenced in such a way that a reduction or increase of the reaction rate or a modification (reduction or increase) of the affinity to the substrate results, is reached. A mutation in the catalytic centre of an polypeptide of the invention, e.g. as enzyme, can modulate the turn over rate of the enzyme, e.g. a knock out of an essential amino acid can lead to a reduced or completely knock out activity of the enzyme, or the deletion or mutation of regulator binding sites can reduce a negative regulation like a feedback inhibition (or a substrate inhibition, if the substrate level is also increased). The specific activity of an enzyme of the present invention can be increased such that the turn over rate is increased or the binding of a co-factor is improved. Improving the stability of the encoding mRNA or the protein can also increase the activity of a gene product. The stimulation of the activity is also under the scope of the term “increased activity”.

Moreover, the regulation of the abovementioned nucleic acid sequences may be modified so that gene expression is increased. This can be achieved advantageously by means of heterologous regulatory sequences or by modifying, for example mutating, the natural regulatory sequences which are present. The advantageous methods may also be combined with each other.

In general, an activity of a gene product in an organism or part thereof, in particular in a plant cell or organelle of a plant cell, a plant, or a plant tissue or a part thereof or in a microorganism can be increased by increasing the amount of the specific encoding mRNA or the corresponding protein in said organism or part thereof.

A modification, i.e. an increase, can be caused by endogenous or exogenous factors. For example, an increase in activity in an organism or a part thereof can be caused by adding a gene product or a precursor or an activator or an agonist to the media or nutrition or can be caused by introducing said subjects into a organism, transient or stable. Furthermore such an increase can be reached by the introduction of the inventive nucleic acid sequence or the encoded protein in the correct cell compartment for example into the nucleus or cytoplasm respectively or into plastids either by transformation and/or targeting.

In one further embodiment of the process according to the invention, organisms are used in which one of the abovementioned genes, or one of the abovementioned nucleic acids, is mutated in a way that the activity of the encoded gene products is less influenced by cellular factors, or not at all, in comparison with the not mutated proteins. For example, well known regulation mechanisms of enzyme activity are substrate inhibition or feed back regulation mechanisms. Ways and techniques for the introduction of substitution, deletions and additions of one or more bases, nucleotides or amino acids of a corresponding sequence are described herein below in the corresponding paragraphs and the references listed there, e.g. in Sambrook et al., Molecular Cloning, Cold Spring Harbour, N.Y., 1989. The person skilled in the art will be able to identify regulation domains and binding sites of regulators by comparing the sequence of the nucleic acid molecule of the present invention or the expression product thereof with the state of the art by computer software means which comprise algorithms for the identifying of binding sites and regulation domains or by introducing into a nucleic acid molecule or in a protein systematically mutations and assaying for those mutations which will lead to an increased specific activity or an increased activity per volume, in particular per cell.

It can therefore be advantageous to express in an organism a nucleic acid molecule of the invention or a polypeptide of the invention derived from a evolutionary distantly related organism, as e.g. using a prokaryotic gene in a eukaryotic host, as in these cases the regulation mechanism of the host cell may not weaken the activity (cellular or specific) of the gene or its expression product.

The mutation is introduced in such a way that increased herbicide tolerance or resistance, is not adversely affected.

The invention is not limited to specific nucleic acids, specific polypeptides, specific cell types, specific host cells, specific conditions or specific methods etc. as such, but may vary and numerous modifications and variations therein will be apparent to those skilled in the art. It is also to be understood that the terminology used herein is for the purpose of describing specific embodiments only and is not intended to be limiting.

Further, “proteins are generally composed of one or more functional regions, commonly termed domains. Different combinations of domains give rise to the diverse range of proteins found in nature. The identification of domains that occur within proteins can therefore provide insights into their function. Pfam-A entries are high quality, manually curated families. The Pfam database is a large collection of protein families, each represented by multiple sequence alignments and hidden Markov models (HMMs).” (see: The Pfam protein families database: R. D. Finn, et al., Nucleic Acids Research (2010), Database Issue 38:D211-222). The Pfam protein family database is a large collection of more than ten thousand protein families and is available under http://pfam.sanger.ac.uk/. Profile Hidden Markov Models (HMMs) are flexible, probabilistic models that can be used to describe the consensus patterns shared by sets of homologous protein/domain sequences. HMMs in the Pfam database are constructed from an alignment of a representative set of sequences for each protein domain, called a seed alignment.

Accordingly, the present invention relates to a nucleic acid molecule encoding a polypeptide which is 50% or more, preferably 60%, 70%, or 75%, more preferably 80%, 85%, 90%, or 95%, even more preferred 96%, 97%, 98%, 99% or more and most preferred 100% identical to the polypeptide of SEQ ID NO: 2, 4, 6, 8, 27, or 45, and conferring the increase of the herbicide tolerance or resistance of a plant as described herein. The invention also relates to the polypeptide encoded by said polynucleotide.

The present invention also relates to isolated nucleic acids comprising a nucleic acid molecule selected from the group consisting of:
(a) a nucleic acid molecule encoding the polypeptide comprising the sequence of SEQ ID NO: 2, 4, 6, 8, 27, or 45, or a homolog thereof;
(b) a nucleic acid molecule comprising the sequence of SEQ ID NO: 1, 3, 5, 7, 26, or 44, or a homolog thereof,
(c) a nucleic acid molecule, which, as a result of the degeneracy of the genetic code, can be derived from a polypeptide sequence of SEQ ID NO: 2, 4, 6, 8, 27, or 45, or a homolog thereof, and confers increased herbicide tolerance or resistance, as compared to a corresponding, e.g. non-transformed, wild type plant cell, a plant or a part thereof;
(d) a nucleic acid molecule having 30% or more identity, preferably 40%, 50%, 60%, 70%, 75%, 80%, 85%, 90%, 95%, 96%, 97%, 98%, 99%, 99.5%, or more with the nucleic acid molecule sequence of a polynucleotide comprising the nucleic acid molecule of SEQ ID NO: 1, 3, 5, 7, 26, or 44, or a homolog thereof, and confers increased herbicide tolerance or resistance, as compared to a corresponding, e.g. non-transformed, wild type plant cell, a plant or a part thereof;
(e) a nucleic acid molecule encoding a polypeptide having 30% or more identity, preferably at least 40%, 50%, 60%, 70%, 75%, 80%, 85%, 90%, 95%, 96%, 97%, 98%, 99%, 99.5% or more, with the amino acid sequence of the polypeptide encoded by the nucleic acid molecule of (a), (b), (c) or (d) and having the activity represented by a nucleic acid molecule comprising a polynucleotide of SEQ ID NO: 1, 3, 5, 7, 26, or 44, or a homolog thereof, and confers increased herbicide tolerance or resistance as compared to a corresponding, e.g. non-transformed, wild type plant cell, a plant or a part thereof;
(f) nucleic acid molecule which hybridizes with a nucleic acid molecule of (a), (b), (c), (d) or (e) under stringent hybridization conditions and confers increased herbicide tolerance or resistance, as compared to a corresponding, e.g. non-transformed, wild type plant cell, a plant or a part thereof;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f) and having the activity represented by the nucleic acid molecule comprising a polynucleotide as depicted in SEQ ID NO: 1, 3, 5, 7, 26, or 44, or a homolog thereof;
(h) a nucleic acid molecule which is obtainable by screening a suitable nucleic acid library, especially a cDNA library and/or a genomic library, under stringent hybridization conditions with a probe comprising a complementary sequence of a nucleic acid molecule of (a) or (b) or with a fragment thereof, having 15 nt, preferably 20 nt, 30 nt, 50 nt, 100 nt, 200 nt, 500 nt, 750 nt or 1000 nt or more of a nucleic acid molecule complementary to a nucleic acid molecule sequence characterized in (a) to (e) and encoding a polypeptide having the activity represented by a protein comprising a polypeptide as depicted SEQ ID NO: 2, 4, 6, 8, 27, or 45, or a homolog thereof.

In one embodiment, the nucleic acid molecule according to (a), (b), (c), (d), (e), (f), (g), (h), is at least in one or more nucleotides different from the sequence depicted in SEQ ID NO: 1, 3, 5, 7, 26, or 44, and encodes a protein which differs at least in one or more amino acids from the protein sequences depicted in SEQ ID NO: 2, 4, 6, 8, 27, or 45.

In one embodiment the invention relates to homologs of the aforementioned sequences, which can be isolated advantageously from yeast, fungi, viruses, algae, bacteria, such as Acetobacter (subgen. Acetobacter) aceti; Acidithiobacillus ferrooxidans; Acinetobacter sp.; Actinobacillus sp; Aeromonas salmonicida; Agrobacterium tumefaciens; Aquifex aeolicus; Arcanobacterium pyogenes; Aster yellows phytoplasma; Bacillus sp.; Bifidobacterium sp.; Borrelia burgdorferi; Brevibacterium linens; Brucella melitensis; Buchnera sp.; Butyrivibrio fibrisolvens; Campylobacter jejuni; Caulobacter crescentus; Chlamydia sp.; Chlamydophila sp.; Chlorobium limicola; Citrobacter rodentium; Clostridium sp.; Comamonas testosteroni; Corynebacterium sp.; Coxiella bumetii; Deinococcus radiodurans; Dichelobacter nodosus; Edwardsiella ictaluri; Enterobacter sp.; Erysipelothrix rhusiopathiae; E coli; Flavobacterium sp.; Francisella tularensis; Frankia sp. Cpl1; Fusobacterium nucleatum; Geobacillus stearothermophilus; Gluconobacter oxydans; Haemophilus sp.; Helicobacter pylori; Klebsiella pneumoniae; Lactobacillus sp.; Lactococcus lactis; Listeria sp.; Mannheimia haemolytica; Mesorhizobium loti; Methylophaga thalassica; Microcystic aeruginosa; Microscilla sp. PRE1; Moraxella sp. TA 144; Mycobacterium sp.; Mycoplasma sp.; Neisseria sp.; Nitrosomonas sp.; Nostoc sp. PCC 7120; Novosphingobium aromaticivorans; Oenococcus oeni; Pantoea citrea; Pasteurella multocida; Pediococcus pentosaceus; Phormidium foveolarum; Phytoplasma sp.; Plectonema boryanum; Prevotella ruminicola; Propionibacterium sp.; Proteus vulgaris; Pseudomonas sp.; Ralstonia sp.; Rhizobium sp.; Rhodococcus equi; Rhodothermus marinus; Rickettsia sp.; Riemerella anatipestifer; Ruminococcus flavefaciens; Salmonella sp.; Selenomonas ruminantium; Serratia entomophila; Shigella sp.; Sinorhizobium meliloti; Staphylococcus sp.; Streptococcus sp.; Streptomyces sp.; Synechococcus sp.; Synechocystis sp. PCC 6803; Thermotoga maritima; Treponema sp.; Ureaplasma urealyticum; Vibrio cholerae; Vibrio parahaemolyticus; Xylella fastidiosa; Yersinia sp.; Zymomonas mobllis, preferably Salmonella sp. or E. coli or plants, preferably from yeasts such as from the genera Saccharomyces, Pichia, Candida, Hansenula, Torulopsis or Schizosaccharomyces or plants such as A. thaliana, maize, wheat, rye, oat, triticale, rice, barley, soybean, peanut, cotton, borage, sunflower, linseed, primrose, rapeseed, canola and turnip rape, manihot, pepper, sunflower, tagetes, solanaceous plant such as potato, tobacco, egg-plant and tomato, Vicia species, pea, alfalfa, bushy plants such as coffee, cacao, tea, Salix species, trees such as oil palm, coconut, perennial grass, such as ryegrass and fescue, and forage crops, such as alfalfa and clover and from spruce, pine or fir for example. More preferably homologs of aforementioned sequences can be isolated from S. cerevisiae, E coli or Synechocystis sp. or plants, preferably Brassica napus, Glycine max, Zea mays, cotton or Oryza sativa. In a particularly preferred embodiment, the homolog refers to a polypeptide comprising the sequence of SEQ ID NO: 9, 10, 11, 12, 13, 14, 15, 16, 17, 18, 19, 20, 21, 22, 23, 24, 25, 28, 29, 30, 31, 32, 33, 34, 35, 36, 37, 38, 39, 40, 41, 42, 43, 46, 47, 48, 49, 50, 51, 52, 53, 54, 55, 56, 57, 58, 59, 60, 61, 62, 63, 64, 65, 66, 67, 68, or 69.

The proteins of the present invention are preferably produced by recombinant DNA techniques. For example, a nucleic acid molecule encoding the protein is cloned into an expression vector, for example in to a binary vector, the expression vector is introduced into a host cell, for example the A. thaliana wild type NASC N906 or any other plant cell as described in the examples see below, and the protein is expressed in said host cell. Examples for binary vectors are pBIN19, pBI101, pBinAR (Höfgen and Willmitzer, Plant Science 66, 221 (1990)), pGPTV, pCAMBIA, pBIB-HYG, pBecks, pGreen or pPZP (Hajukiewicz, P. et al., Plant Mol. Biol. 25, 989 (1994), and Hellens et al, Trends in Plant Science 5, 446 (2000)).

In one embodiment as described in more detail SUPRA, the protein of the present invention is preferably targeted to an compartment of the cell, e.g. to the endoplasmic reticulum or in the plastids. Ways of introducing nucleic acids into the endoplasmic reticulum or plastids and producing proteins in this compartment are known to the person skilled in the art have been also described in this application. In one embodiment, the polypeptide of the invention is a protein localized after expression e.g. non-targeted, mitochondrial or plastidic, for example it is fused to a transit or signal sequence peptide as described above for plastidic or endoplasmic reticulum localisation. In another embodiment the protein of the present invention is produced without further targeting signal (e.g. as mentioned herein), e.g. in the cytoplasm of the cell. Ways of producing proteins in the cytoplasm are known to the person skilled in the art. Ways of producing proteins without artificial targeting are known to the person skilled in the art.

Advantageously, the nucleic acid sequences according to the invention or the gene construct together with at least one reporter gene are cloned into an expression cassette, which is introduced into the organism via a vector or directly into the genome. This reporter gene should allow easy detection via a growth, fluorescence, chemical, bioluminescence or tolerance assay or via a photometric measurement. Examples of reporter genes which may be mentioned are antibiotic- or herbicide-tolerance genes, hydrolase genes, fluorescence protein genes, bioluminescence genes, sugar or nucleotide metabolic genes or biosynthesis genes such as the Ura3 gene, the Ilv2 gene, the luciferase gene, the β-galactosidase gene, the gfp gene, the 2-desoxyglucose-6-phosphate phosphatase gene, the β-glucuronidase gene, β-lactamase gene, the neomycin phosphotransferase gene, the hygromycin phosphotransferase gene, a mutated acetohydroxyacid synthase (AHAS) gene (also known as acetolactate synthase (ALS) gene), a gene for a D-amino acid metabolizing enzmye or the BASTA (=gluphosinate-tolerance) gene. These genes permit easy measurement and quantification of the transcription activity and hence of the expression of the genes. In this way genome positions may be identified which exhibit differing productivity. For expression a person skilled in the art is familiar with different methods to introduce the nucleic acid sequences into different organelles such as the preferred plastids. Such methods are for example disclosed by Maiga P. (Annu. Rev. Plant Biol. 55, 289 (2004)), Evans T. (WO 2004/040973), McBride K. E. et al. (U.S. Pat. No. 5,455,818), Daniell H. et al. (U.S. Pat. No. 5,932,479 and U.S. Pat. No. 5,693,507) and Straub J. M. et al. (U.S. Pat. No. 6,781,033). A preferred method is the transformation of microspore-derived hypocotyl or cotyledonary tissue (which are green and thus contain numerous plastids) leaf tissue and afterwards the regeneration of shoots from said transformed plant material on selective medium. As methods for the transformation bombarding of the plant material or the use of independently replicating shuttle vectors are well known by the skilled worker. But also a PEG-mediated transformation of the plastids or Agrobacterium transformation with binary vectors is possible. Useful markers for the transformation of plastids are positive selection markers for example the chloramphenicol-, streptomycin-, kanamycin-, neomycin-, amikamycin-, spectinomycin-, triazine- and/or lincomycin-tolerance genes. As additional markers named in the literature often as secondary markers, genes coding for the tolerance against herbicides such as phosphinothricin (=glufosinate, BASTA™, Liberty™, encoded by the bar gene), glyphosate (═N-(phosphonomethyl)glycine, Roundup™, encoded by the 5-enolpyruvylshikimate-3-phosphate synthase gene=epsps), sulfonylureas (like Staple™, encoded by the acetolactate synthase (ALS) gene), imidazolinones [=IMI, like imazethapyr, imazamox, Clearfield™, encoded by the acetohydroxyacid synthase (AHAS) gene, also known as acetolactate synthase (ALS) gene] or bromoxynil (=Buctril™, encoded by the oxy gene) or genes coding for antibiotics such as hygromycin or G418 are useful for further selection. Such secondary markers are useful in the case when most genome copies are transformed. In addition negative selection markers such as the bacterial cytosine deaminase (encoded by the codA gene) are also useful for the transformation of plastids.

To increase the possibility of identification of transformants it is also desirable to use reporter genes other then the aforementioned tolerance genes or in addition to said genes. Reporter genes are for example β-galactosidase-, β-glucuronidase-(GUS), alkaline phosphatase- and/or green-fluorescent protein-genes (GFP).

In a preferred embodiment a nucleic acid construct, for example an expression cassette, comprises upstream, i.e. at the 5′ end of the encoding sequence, a promoter and downstream, i.e. at the 3′ end, a polyadenylation signal and optionally other regulatory elements which are operably linked to the intervening encoding sequence with one of the nucleic acids of SEQ ID NO: 1, 3, 5, 7, 26, or 44, or a homolog thereof. By an operable linkage is meant the sequential arrangement of promoter, encoding sequence, terminator and optionally other regulatory elements in such a way that each of the regulatory elements can fulfill its function in the expression of the encoding sequence in due manner. In one embodiment the sequences preferred for operable linkage are targeting sequences for ensuring sub-cellular localization in plastids. However, targeting sequences for ensuring subcellular localization in the mitochondrium, in the endoplasmic reticulum (=ER), in the nucleus, in oil corpuscles or other compartments may also be employed as well as translation promoters such as the 5′ lead sequence in tobacco mosaic virus (Gallie et al., Nucl. Acids Res. 15 8693 (1987).

A nucleic acid construct, for example an expression cassette may, for example, contain a constitutive promoter or a tissue-specific promoter (preferably the USP or napin promoter) the gene to be expressed and the ER retention signal. For the ER retention signal the KDEL amino acid sequence (lysine, aspartic acid, glutamic acid, leucine) or the KKX amino acid sequence (lysine-lysine-X-stop, wherein X means every other known amino acid) is preferably employed.

For expression in a host organism, for example a plant, the expression cassette is advantageously inserted into a vector such as by way of example a plasmid, a phage or other DNA which allows optimal expression of the genes in the host organism. Examples of suitable plasmids are: in E. coli pLG338, pACYC184, pBR series such as e.g. pBR322, pUC series such as pUC18 or pUC19, M113 mp series, pKC30, pRep4, pHS1, pHS2, pPLc236, pMBL24, pLG200, pUR290, pIN-111113-B1, λgt11 or pBdCl; in Streptomyces pIJ101, pIJ364, pIJ702 or pIJ361; in Bacillus pUB110, pC194 or pBD214; in Corynebacterium pSA77 or pAJ667; in fungi pALS1, pIL2 or pBB116; other advantageous fungal vectors are described by Romanos M. A. et al., Yeast 8, 423 (1992) and by van den Hondel, C. A. M. J. J. et al. [(1991) “Heterologous gene expression in filamentous fungi”] as well as in “More Gene Manipulations” in “Fungi” in Bennet J. W. & Lasure L. L., eds., pp. 396-428, Academic Press, San Diego, and in “Gene transfer systems and vector development for filamentous fungi” [van den Hondel, C. A. M. J. J. & Punt, P. J. (1991) in: Applied Molecular Genetics of Fungi, Peberdy, J. F. et al., eds., pp. 1-28, Cambridge University Press: Cambridge]. Examples of advantageous yeast promoters are 2 μM, pAG-1, YEp6, YEp13 or pEMBLYe23. Examples of algal or plant promoters are pLGV23, pGHIac+, pBIN19, pAK2004, pVKH or pDH51 (see Schmidt, R. and Willmitzer, L., Plant Cell Rep. 7, 583 (1988)). The vectors identified above or derivatives of the vectors identified above are a small selection of the possible plasmids. Further plasmids are well known to those skilled in the art and may be found, for example, in “Cloning Vectors” (Eds. Pouwels P. H. et al. Elsevier, Amsterdam-N.Y.-Oxford, 1985, ISBN 0 444 904018). Suitable plant vectors are described inter alia in “Methods in Plant Molecular Biology and Biotechnology” (CRC Press, Ch. 6/7, pp. 71-119). Advantageous vectors are known as shuttle vectors or binary vectors which replicate in E. coli and Agrobacterium.

In a further embodiment of the vector the expression cassette according to the invention may also advantageously be introduced into the organisms in the form of a linear DNA and be integrated into the genome of the host organism by way of heterologous or homologous recombination. This linear DNA may be composed of a linearized plasmid or only of the expression cassette as vector or the nucleic acid sequences according to the invention.

A nucleic acid sequence can also be introduced into an organism on its own.

If in addition to the nucleic acid sequence according to the invention further genes are to be introduced into the organism, all together with a reporter gene in a single vector or each single gene with a reporter gene in a vector in each case can be introduced into the organism, whereby the different vectors can be introduced simultaneously or successively.

The vector advantageously contains at least one copy of the nucleic acid sequences according to the invention and/or the expression cassette (=gene construct) according to the invention.

The invention further provides an isolated recombinant expression vector comprising a nucleic acid encoding a polypeptide comprising the sequence of SEQ ID NO: 2, 4, 6, 8, 27, or 45, or a homolog thereof, wherein expression of the vector in a host cell results in increased herbicide tolerance or resistance, as compared to a wild type variety of the host cell.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. It will be appreciated by those skilled in the art that the design of the expression vector can depend on such factors as the choice of the host cell to be transformed, the level of expression of polypeptide desired, etc. The expression vectors of the invention can be introduced into host cells to thereby produce polypeptides or peptides, including fusion polypeptides or peptides, encoded by nucleic acids as described herein.

The recombinant expression vectors of the invention can be designed for expression of the polypeptide of the invention in plant cells. For example, nucleic acid molecules of the present invention can be expressed in plant cells (see Schmidt R., and Willmitzer L., Plant Cell Rep. 7 (1988); Plant Molecular Biology and Biotechnology, C Press, Boca Raton, Fla., Chapter 6/7, p. 71-119 (1993); White F. F., Jenes B. et al., Techniques for Gene Transfer, in: Transgenic Plants, Vol. 1, Engineering and Utilization, eds. Kung and Wu R., 128-43, Academic Press: 1993; Potrykus, Annu. Rev. Plant Physiol. Plant Molec. Biol. 42, 205 (1991) and references cited therein). Suitable host cells are discussed further in Goeddel, Gene Expression Technology: Methods in Enzymology 185, Academic Press: San Diego, Calif. (1990). By way of example the plant expression cassette can be installed in the pRT transformation vector ((a) Toepfer et al., Methods Enzymol. 217, 66 (1993), (b) Toepfer et al., Nucl. Acids. Res. 15, 5890 (1987)). Alternatively, a recombinant vector (=expression vector) can also be transcribed and translated in vitro, e.g. by using the T7 promoter and the T7 RNA polymerase.

In an further embodiment of the present invention, the nucleic acid molecules of the invention are expressed in plants and plants cells such as unicellular plant cells (e.g. algae) (see Falciatore et al., Marine Biotechnology 1 (3), 239 (1999) and references therein) and plant cells from higher plants (e.g., the spermatophytes, such as crop plants), for example to regenerate plants from the plant cells. A nucleic acid molecule depicted in SEQ ID NO: 1, 3, 5, 7, 26, or 44, or a homolog thereof may be “introduced” into a plant cell by any means, including transfection, transformation or transduction, electroporation, particle bombardment, agroinfection, and the like. One transformation method known to those of skill in the art is the dipping of a flowering plant into an Agrobacteria solution, wherein the Agrobacteria contains the nucleic acid of the invention, followed by breeding of the transformed gametes. Other suitable methods for transforming or transfecting host cells including plant cells can be found in Sambrook et al., supra, and other laboratory manuals such as Methods in Molecular Biology, 1995, Vol. 44, Agrobacterium protocols, ed: Gartland and Davey, Humana Press, Totowa, N.J.

In one embodiment of the present invention, transfection of a nucleic acid molecule coding for a polypeptide comprising the sequence of SEQ ID NO: 2, 4, 6, 8, 27, or 45, or a homolog thereof into a plant is achieved by Agrobacterium mediated gene transfer. Agrobacterium mediated plant transformation can be performed using for example the GV3101(pMP90) (Koncz and Schell, Mol. Gen. Genet. 204, 383 (1986)) or LBA4404 (Clontech) Agrobacterium tumefaciens strain. Transformation can be performed by standard transformation and regeneration techniques (Deblaere et al., Nucl. Acids Res. 13, 4777 (1994), Gelvin, Stanton B. and Schilperoort Robert A, Plant Molecular Biology Manual, 2nd Ed.—Dordrecht: Kluwer Academic Publ., 1995.—in Sect., Ringbuc Zentrale Signatur: BT11-P ISBN 0-7923-2731-4; Glick Bernard R., Thompson John E., Methods in Plant Molecular Biology and Biotechnology, Boca Raton: CRC Press, 1993 360 S., ISBN 0-8493-5164-2). For example, rapeseed can be transformed via cotyledon or hypocotyl transformation (Moloney et al., Plant Cell Report 8, 238 (1989); De Block et al., Plant Physiol. 91, 694 (1989)). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., Plant Cell Report 13, 282 (1994). Additionally, transformation of soybean can be performed using for example a technique described in European Patent No. 424 047, U.S. Pat. No. 5,322,783, European Patent No. 397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique. (see, for example, Freeling and Walbot “The maize handbook” Springer Verlag: New York (1993) ISBN 3-540-97826-7). A specific example of maize transformation is found in U.S. Pat. No. 5,990,387, and a specific example of wheat transformation can be found in PCT Application No. WO 93/07256.

According to the present invention, the introduced nucleic acid molecule coding for a polypeptides depicted in SEQ ID NO: 2, 4, 6, 8, 27, or 45, or homologs thereof, may be maintained in the plant cell stably if it is incorporated into a non-chromosomal autonomous replicon or integrated into the plant chromosomes or organelle genome. Alternatively, the introduced nucleic acid molecule may be present on an extra-chromosomal non-replicating vector and be transiently expressed or transiently active.

In one embodiment, a homologous recombinant microorganism can be created wherein the nucleic acid molecule is integrated into a chromosome, a vector is prepared which contains at least a portion of a nucleic acid molecule coding for a protein depicted in SEQ ID NO: 2, 4, 6, 8, 27, or 45, or a homolog thereof into which a deletion, addition, or substitution has been introduced to thereby alter, e.g., functionally disrupt, the gene. For example, the gene is a yeast gene, like a gene of S. cerevisiae, or of Synechocystis, or a bacterial gene, like an E. coli gene, but it can be a homolog from a related plant or even from a mammalian or insect source. The vector can be designed such that, upon homologous recombination, the endogenous nucleic acid molecule coding for a protein depicted in SEQ ID NO: 2, 4, 6, 8, 27, or 45, or a homolog thereof is mutated or otherwise altered but still encodes a functional polypeptide (e.g., the upstream regulatory region can be altered to thereby alter the expression of the endogenous nucleic acid molecule). In a preferred embodiment the biological activity of the protein of the invention is increased upon homologous recombination. To create a point mutation via homologous recombination, DNA-RNA hybrids can be used in a technique known as chimeraplasty (Cole-Strauss et al., Nucleic Acids Research 27 (5), 1323 (1999) and Kmiec, Gene Therapy American Scientist. 87 (3), 240 (1999)). Homologous recombination procedures in Physcomitrella patens are also well known in the art and are contemplated for use herein.

Whereas in the homologous recombination vector, the altered portion of the nucleic acid molecule coding for a protein depicted in SEQ ID NO: 2, 4, 6, 8, 27, or 45, or a homolog thereof is flanked at its 5′ and 3′ ends by an additional nucleic acid molecule of the gene to allow for homologous recombination to occur between the exogenous gene carried by the vector and an endogenous gene, in a microorganism or plant. The additional flanking nucleic acid molecule is of sufficient length for successful homologous recombination with the endogenous gene. Typically, several hundreds of base pairs up to kilobases of flanking DNA (both at the 5′ and 3′ ends) are included in the vector. See, e.g., Thomas K. R., and Capecchi M. R., Cell 51, 503 (1987) for a description of homologous recombination vectors or Strepp et al., PNAS, 95 (8), 4368 (1998) for cDNA based recombination in Physcomitrella patens. The vector is introduced into a microorganism or plant cell (e.g. via polyethylene glycol mediated DNA), and cells in which the introduced gene has homologously recombined with the endogenous gene are selected using art-known techniques.

Whether present in an extra-chromosomal non-replicating vector or a vector that is integrated into a chromosome, the nucleic acid molecule coding for amino acid molecules depicted in SEQ ID NO: 2, 4, 6, 8, 27, or 45, or a homolog thereof preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., EMBO J. 3, 835 (1984)) or functional equivalents thereof but also all other terminators functionally active in plants are suitable. As plant gene expression is very often not limited on transcriptional levels, a plant expression cassette preferably contains other operatively linked sequences like translational enhancers such as the overdrive-sequence containing the 5″-untranslated leader sequence from tobacco mosaic virus enhancing the polypeptide per RNA ratio (Gallie et al., Nucl. Acids Research 15, 8693 (1987)). Examples of plant expression vectors include those detailed in: Becker D. et al., Plant Mol. Biol. 20, 1195 (1992); and Bevan M. W., Nucl. Acid. Res. 12, 8711 (1984); and “Vectors for Gene Transfer in Higher Plants” in: Transgenic Plants, Vol. 1, Engineering and Utilization, eds. Kung and Wu R., Academic Press, 1993, S. 15-38.

The host organism (=transgenic organism) advantageously contains at least one copy of the nucleic acid according to the invention and/or of the nucleic acid construct according to the invention.

In principle all plants can be used as host organism. Preferred transgenic plants are, for example, selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants such as plants advantageously selected from the group of the genus peanut, oilseed rape, canola, sunflower, safflower, olive, sesame, hazelnut, almond, avocado, bay, pumpkin/squash, linseed, soya, pistachio, borage, maize, wheat, rye, oats, sorghum and millet, triticale, rice, barley, cassava, potato, sugarbeet, egg plant, alfalfa, and perennial grasses and forage plants, oil palm, vegetables (brassicas, root vegetables, tuber vegetables, pod vegetables, fruiting vegetables, onion vegetables, leafy vegetables and stem vegetables), buckwheat, Jerusalem artichoke, broad bean, vetches, lentil, dwarf bean, lupin, clover and Lucerne for mentioning only some of them.

In one embodiment of the invention transgenic plants are selected from the group comprising cereals, soybean, rapeseed (including oil seed rape, especially canola and winter oil seed rape), cotton, sugarcane, sugar beet and potato, especially corn, soy, rapeseed (including oil seed rape, especially canola and winter oil seed rape), cotton, wheat and rice.

In another embodiment of the invention the transgenic plant is a gymnosperm plant, especially a spruce, pine or fir.

In one embodiment, the host plant is selected from the families Aceraceae, Anacardiaceae, Apiaceae, Asteraceae, Brassicaceae, Cactaceae, Cucurbitaceae, Euphorbiaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, Asteraceae, Brassicaceae, Cucurbitaceae, Fabaceae, Papaveraceae, Rosaceae, Solanaceae, Liliaceae or Poaceae. Preferred are crop plants and in particular plants mentioned herein above as host plants such as the families and genera mentioned above for example preferred the species Anacardium ocadentale, Calendula officinalis, Carthamus tinctorius, Cichorium intybus, Cynara scolymus, Helianthus annus, Tagetes lucida, Tagetes erecta, Tagetes tenuifolia; Daucus carota; Corylus avellana, Corylus colurea, Borago officinalis; Brassica napus, Brassica rapa ssp., Sinapis arvensis Brassica juncea, Brassica juncea var. juncea, Brassica juncea var. crispifolia, Brassica juncea var. foliosa, Brassica nigra, Brassica sinapiodes, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fastigiata, Ipomoea tiliacea, Ipomoea triloba, Convolvulus panduratus, Beta vulgaris, Beta vulgaris var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot, Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago vana, Glycine max Dolichos soja, Glycine gracilis, Glycine hispida, Phaseolus max, Soja hispida, Soja max, Cocos nucifera, Pelargonium grossulariodes, Oleum cocoas, Laurus nobilis, Persea americana, Arachis hypogaea, Linum usitatissimum, Linum humile, Linum austnacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientate, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Triticum vulgare, Cofea spp., Coffea arabica, Coffea canephora, Coffea liberica, Capsicum annuum, Capsicum annuum var glabriusculum, Capsicum frutescens, Capsicum annuum, Nicotiana tabacum, Solanum tuberosum, Solanum melongena, Lycopersicon esculentum, Lycopersicon lycopersicum, Lycopersicon pyriforme, Solanum integrifolium, Solanum lycopersicum Theobroma cacao or Camellia sinensis.

Anacardiaceae such as the genera Pistacia, Mangifera, Anacardium e.g. the species Pistacia vera [pistachios, Pistazie], Mangifer indica [Mango] or Anacardium ocadentale [Cashew]; Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [cornflower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. roman, Locusta communis, Valerian locusta [lettuce], Tagetes lucida, Tagetes erecta or Tagetes tenuifolia[Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinails [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var. juncea, Brassica juncea var. crispfolia, Brassica juncea var. foliosa, Brassica nigra, Brassica sinapiodes, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatus, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var. Vulgaris, Beta maritima, Beta vulgaris var. perennis, Beta vulgaris var conditiva or Beta vulgaris var. esculenta [sugar beet]; Cucurbitaceae such as the genera Cucubita e.g. the species Cucurbita maxima, Cucurbita mixta, Cucurbita pepo or Cucurbita moschata [pumpkin, squash]; Elaeagnaceae such as the genera Elaeagnus e.g. the species Olea europaea [olive]; Ericaceae such as the genera Kalmia e.g. the species Kalmia latifolia, Kalmia augustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot, Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellobium berterianum, Pithecellobium fragrans, Pithecolobium berterianum, Pseudalbizzia berteriana, Acacia julibrissin, Acacia nemu, Albizia nemu, Feuilleea jufibrissin, Mimosa julibrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa] Glycine max Dolichos soja, Glycine gracilis, Glycine hispida, Phaseolus max, Soja hispida or Soja max [soybean]; Geraniaceae such as the genera Pelargonium, Cocos, Oleum e.g. the species Cocos nucifera, Pelargonium grossulariodes or Oleum cocois [coconut]; Gramineae such as the genera Saccharum e.g. the species Saccharum officinarum; Juglandaceae such as the genera Juglans, Wallia e.g. the species Juglans regia, Juglans allanthifolia, Juglans sieboldiana, Juglans cinerea, Wallia cinerea, Juglans bixbyi, Juglans californica, Juglans hindsii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adenolinum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oenothera biennis or Camissonia brevipes [primrose, evening primrose]; Palmae such as the genera Elacis e.g. the species Elaeis guineensis [oil plam]; Papaveraceae such as the genera Papaver e.g. the species Papaver orientate, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Artanthe elongata, Peperomia elongata, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow barley], Secale cereale [rye], Avena sativa, Avena fatua, Avena byzantina, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum [Sorghum, millet], Oryza sativa, Oryza latifolia [rice], Zea mays [corn, maize] Triticum aestivum, Triticum durum, Triticum turgidum, Triticum hybernum, Triticum macha, Triticum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phiomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuate, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant] (Lycopersicon esculentum, Lycopersicon lycopersicum, Lycopersicon pyriforme, Solanum integrifolium or Solanum lycopersicum [tomato]; Sterculiaceae such as the genera Theobroma e.g. the species Theobroma cacao [cacao]; Theaceae such as the genera Camellia e.g. the species Camellia sinensis) [tea].

The introduction of the nucleic acids according to the invention, the expression cassette or the vector into organisms, plants for example, can in principle be done by all of the methods known to those skilled in the art. The introduction of the nucleic acid sequences gives rise to recombinant or transgenic organisms.

The transfer of foreign genes into the genome of a plant is called transformation. In doing this the methods described for the transformation and regeneration of plants from plant tissues or plant cells are utilized for transient or stable transformation. Suitable methods are protoplast transformation by poly(ethylene glycol)-induced DNA uptake, the “biolistic” method using the gene cannon—referred to as the particle bombardment method, electroporation, the incubation of dry embryos in DNA solution, microinjection and gene transfer mediated by Agrobacterium. Said methods are described by way of example in Jenes B. et al., Techniques for Gene Transfer, in: Transgenic Plants, Vol. 1, Engineering and Utilization, eds. Kung S. D and Wu R., Academic Press (1993) 128-143 and in Potrykus, Annu. Rev. Plant Physiol. Plant Molec. Biol. 42, 205 (1991). The nucleic acids or the construct to be expressed is preferably cloned into a vector which is suitable for transforming Agrobacterium tumefaciens, for example pBin19 (Bevan et al., Nucl. Acids Res. 12, 8711 (1984)). Agrobacteria transformed by such a vector can then be used in known manner for the transformation of plants, in particular of crop plants such as by way of example tobacco plants, for example by bathing bruised leaves or chopped leaves in an agrobacterial solution and then culturing them in suitable media. The transformation of plants by means of Agrobacterium tumefaciens is described, for example, by Höfgen and Willmitzer in Nucl. Acid Res. 16, 9877 (1988) or is known inter alia from White F. F., Vectors for Gene Transfer in Higher Plants; in Transgenic Plants, Vol. 1, Engineering and Utilization, eds. Kung S. D. and Wu R., Academic Press, 1993, pp. 15-38.

Agrobacteria transformed by an expression vector according to the invention may likewise be used in known manner for the transformation of plants such as test plants like Arabidopsis or crop plants such as cereal crops, corn, oats, rye, barley, wheat, soybean, rice, cotton, sugar beet, canola, sunflower, flax, hemp, potatoes, tobacco, tomatoes, carrots, paprika, oilseed rape, tapioca, cassava, arrowroot, tagetes, alfalfa, lettuce and the various tree, nut and vine species, in particular oil-containing crop plants such as soybean, peanut, castor oil plant, sunflower, corn, cotton, flax, oilseed rape, coconut, oil palm, safflower (Carthamus tinctorius) or cocoa bean, or in particular corn, wheat, soybean, rice, cotton and canola, e.g. by bathing bruised leaves or chopped leaves in an agrobacterial solution and then culturing them in suitable media.

The genetically modified plant cells may be regenerated by all of the methods known to those skilled in the art. Appropriate methods can be found in the publications referred to above by Kung S. D. and Wu R., Potrykus or Höfgen and Willmitzer.

Accordingly, a further aspect of the invention relates to transgenic organisms transformed by at least one nucleic acid sequence, expression cassette or vector according to the invention as well as cells, cell cultures, tissue, parts—such as, for example, leaves, roots, etc. in the case of plant organisms—or reproductive material derived from such organisms.

In one embodiment of the invention host plants for the nucleic acid, expression cassette or vector according to the invention are selected from the group comprising corn, soy, oil seed rape (including canola and winter oil seed rape), cotton, wheat and rice.

A further embodiment of the invention relates to the use of a nucleic acid construct, e.g. an expression cassette, containing one or more DNA sequences encoding one or more polypeptides shown in SEQ ID NO: 2, 4, 6, 8, 27, or 45, or a homolog thereof or comprising one or more nucleic acid molecules as depicted in SEQ ID NO: 1, 3, 5, 7, 26, or 44, or a homolog thereof or encoding or DNA sequences hybridizing therewith for the transformation of plant cells, tissues or parts of plants.

In doing so, depending on the choice of promoter, the nucleic acid molecules of the present invention can be expressed specifically in the leaves, in the seeds, the nodules, in roots, in the stem or other parts of the plant. Those transgenic plants overproducing sequences, e.g. as depicted in SEQ ID NO: 1, 3, 5, 7, 26, or 44, or a homolog thereof, the reproductive material thereof, together with the plant cells, tissues or parts thereof are a further object of the present invention.

The expression cassette or the nucleic acid sequences or construct according to the invention containing nucleic acid molecules or sequences as depicted in SEQ ID NO: 1, 3, 5, 7, 26, or 44, or a homolog thereof can, moreover, also be employed for the transformation of the organisms identified by way of example above such as bacteria, yeasts, filamentous fungi and plants.

Within the framework of the present invention, increased herbicide tolerance or resistance, relates to, for example, the artificially acquired trait of increased herbicide tolerance or resistance, by comparison with the non-genetically modified initial plants e.g. the trait acquired by genetic modification of the target organism, and due to functional overexpression of one or more polypeptide (sequences) of SEQ ID NO: 2, 4, 6, 8, 27, or 45, or a homolog thereof, e.g. encoded by the corresponding nucleic acid molecules as depicted in SEQ ID NO: 1, 3, 5, 7, 26, or 44, and/or homologs, in the organisms according to the invention, advantageously in the transgenic plant according to the invention or produced according to the method of the invention, at least for the duration of at least one plant generation.

A constitutive expression of the polypeptide sequences of SEQ ID NO: 2, 4, 6, 8, 27, or 45, or a homolog thereof, encoded by the corresponding nucleic acid molecule as depicted in SEQ ID NO: 1, 3, 5, 7, 26, or 44, or a homolog thereof is, moreover, advantageous. On the other hand, however, an inducible expression may also appear desirable. Expression of the polypeptide sequences of the invention can be either direct to the cytoplasm or the organelles, preferably the plastids of the host cells, preferably the plant cells.

The activity of the protein encoded by the sequences of SEQ ID NO: 2, 4, 6, 8, 27, or 45, or a homolog thereof, encoded by the corresponding nucleic acid molecule as depicted in SEQ ID NO: 1, 3, 5, 7, 26, or 44, or a homolog thereof can be determined, for example, in vitro as described in EXAMPLE 2. In addition, a functional expression of the sequences of SEQ ID NO: 2, 4, 6, 8, 27, or 45, or a homolog thereof, encoded by the corresponding nucleic acid molecule as depicted in SEQ ID NO: 1, 3, 5, 7, 26, or 44, and/or homologs modified in nature and level and its effect on herbicide tolerance or resistance, but also on the metabolic pathways performance can be tested on test plants in greenhouse trials (see EXAMPLE 3).

An additional object of the invention comprises transgenic organisms such as transgenic plants transformed by an expression cassette containing sequences of as depicted in SEQ ID NO: 1, 3, 5, 7, 26, or 44, or a homolog thereof according to the invention or DNA sequences hybridizing therewith, as well as transgenic cells, tissue, parts and reproduction material of such plants. Particular preference is given in this case to transgenic crop plants such as by way of example barley, wheat, rye, oats, corn, soybean, rice, cotton, sugar beet, oilseed rape and canola, sunflower, flax, hemp, thistle, potatoes, tobacco, tomatoes, tapioca, cassava, arrowroot, alfalfa, lettuce and the various tree, nut and vine species.

In one embodiment of the invention transgenic plants transformed by an expression cassette containing or comprising nucleic acid molecules or sequences as depicted in SEQ ID NO: 1, 3, 5, 7, 26, or 44, or a homolog thereof, according to the invention or DNA sequences hybridizing therewith are selected from the group comprising corn, soy, oil seed rape (including canola and winter oil seed rape), cotton, wheat and rice.

For the purposes of the invention plants are mono- and dicotyledonous plants, mosses or algae, especially plants, for example in one embodiment monocotyledonous plants, or for example in another embodiment dicotyledonous plants. A further refinement according to the invention are transgenic plants as described above which contain a nucleic acid sequence or construct according to the invention or a expression cassette according to the invention.

However, transgenic also means that the nucleic acids according to the invention are located at their natural position in the genome of an organism, but that the sequence, e.g. the coding sequence or a regulatory sequence, for example the promoter sequence, has been modified in comparison with the natural sequence. Preferably, transgenic/recombinant is to be understood as meaning the transcription of one or more nucleic acids or molecules of the invention and being shown in SEQ ID NO: 1, 3, 5, 7, 26, or 44, or a homolog thereof, occurs at a non-natural position in the genome. In one embodiment, the expression of the nucleic acids or molecules is homologous. In another embodiment, the expression of the nucleic acids or molecules is heterologous. This expression can be transiently or of a sequence integrated stably into the genome.

Advantageous inducible plant promoters are by way of example the PRP1 promoter (Ward et al., Plant. Mol. Biol. 22361 (1993)), a promoter inducible by benzenesulfonamide (EP 388 186), a promoter inducible by tetracycline (Gatz et al., Plant J. 2, 397 (1992)), a promoter inducible by salicylic acid (WO 95/19443), a promoter inducible by abscisic acid (EP 335 528) and a promoter inducible by ethanol or cyclohexanone (WO 93/21334). Other examples of plant promoters which can advantageously be used are the promoter of cytoplasmic FBPase from potato, the ST-LSI promoter from potato (Stockhaus et al., EMBO J. 8, 2445 (1989)), the promoter of phosphoribosyl pyrophosphate amidotransferase from Glycine max (see also gene bank accession number U87999) or a nodiene-specific promoter as described in EP 249 676.

Such promoters are known to the person skilled in the art or can be isolated from genes which are induced under the conditions mentioned above. In one embodiment, seed-specific promoters may be used for monocotylodonous or dicotylodonous plants.

In principle all natural promoters with their regulation sequences can be used like those named above for the expression cassette according to the invention and the method according to the invention. Over and above this, synthetic promoters may also advantageously be used. In the preparation of an expression cassette various DNA fragments can be manipulated in order to obtain a nucleotide sequence, which usefully reads in the correct direction and is equipped with a correct reading frame. To connect the DNA fragments (=nucleic acids according to the invention) to one another adaptors or linkers may be attached to the fragments. The promoter and the terminator regions can usefully be provided in the transcription direction with a linker or polylinker containing one or more restriction points for the insertion of this sequence. Generally, the linker has 1 to 10, mostly 1 to 8, preferably 2 to 6, restriction points. In general the size of the linker inside the regulatory region is less than 100 bp, frequently less than 60 bp, but at least 5 bp. The promoter may be both native or homologous as well as foreign or heterologous to the host organism, for example to the host plant. In the 5′-3′ transcription direction the expression cassette contains the promoter, a DNA sequence which shown in SEQ ID NO: 1, 3, 5, 7, 26, or 44, or a homolog thereof and a region for transcription termination. Different termination regions can be exchanged for one another in any desired fashion.

A nucleic acid molecule of the present invention, e.g., a nucleic acid molecule encoding a polypeptide which confers increased herbicide tolerance or resistance, in plants, can be isolated using standard molecular biological techniques and the sequence information provided herein. For example, an A. thaliana polypeptide encoding cDNA can be isolated from a A. thaliana c-DNA library or a Synechocystis sp., Brassica napus, Glycine max, Zea mays or Oryza sativa polypeptide encoding cDNA can be isolated from a Synechocystis sp., Brassica napus, Glycine max, Zea mays or Oryza sativa c-DNA library respectively using all or portion of one of the sequences shown in SEQ ID NO: 1, 3, 5, 7, 26, or 44, or a homolog thereof. Moreover, a nucleic acid molecule encompassing all or a portion of one of the sequences of SEQ ID NO: 1, 3, 5, 7, 26, or 44, or a homolog thereof can be isolated by the polymerase chain reaction using oligonucleotide primers designed based upon this sequence. For example, mRNA can be isolated from plant cells (e.g., by the guanidiniumthiocyanate extraction procedure of Chirgwin et al., Biochemistry 18, 5294 (1979)) and cDNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in SEQ ID NO: 1, 3, 5, 7, 26, or 44, or a homolog thereof. A nucleic acid molecule of the invention can be amplified using cDNA or, alternatively, genomic DNA, as a template and appropriate oligonucleotide primers according to standard PCR amplification techniques. The nucleic acid molecule so amplified can be cloned into an appropriate vector and characterized by DNA sequence analysis. Furthermore, the genes employed in the present invention can be prepared by standard synthetic techniques, e.g., using a commercially available automated DNA synthesizer.

In a embodiment, an isolated nucleic acid molecule of the invention comprises one of the nucleotide sequences or molecules as shown in SEQ ID NO: 1, 3, 5, 7, 26, or 44, or a homolog thereof. Moreover, the nucleic acid molecule of the invention can comprise only a portion of the coding region of one of the sequences or molecules of a nucleic acid as shown in SEQ ID NO: 1, 3, 5, 7, 26, or 44, or a homolog thereof, for example, a fragment which can be used as a probe or primer or a fragment encoding a biologically active portion of a polypeptide-according to invention.

Portions of proteins encoded by the polypeptide according to the invention or a polypeptide encoding nucleic acid molecules of the invention are preferably biologically active portions described herein. As used herein, the term “biologically active portion of” a polypeptide is intended to include a portion, e.g. a domain/motif, of increased herbicide tolerance or resistance, in a plant. To determine whether a polypeptide according to the invention, or a biologically active portion thereof, results in an increased herbicide tolerance or resistance, an analysis of a plant comprising the polypeptide may be performed. Such analysis methods are well known to those skilled in the art, as detailed in the Examples. More specifically, nucleic acid fragments encoding biologically active portions of a polypeptide can be prepared by isolating a portion of one of the sequences of the nucleic acid molecules listed in SEQ ID NO: 1, 3, 5, 7, 26, or 44, or a homolog thereof expressing the encoded portion of the polypeptide or peptide thereof (e.g., by recombinant expression in vitro) and assessing the activity of the encoded portion.

Biologically active portions of the polypeptide according to the invention are encompassed by the present invention and include peptides comprising amino acid sequences derived from the amino acid sequence of the polypeptide encoding gene, or the amino acid sequence of a protein homologous to the polypeptide according to the invention, which include fewer amino acids than a full length polypeptide according to the invention or the full length protein which is homologous to the polypeptide according to the invention, and exhibits at least some enzymatic or biological activity of the polypeptide according to the invention. Typically, biologically active portions (e.g., peptides which are, for example, 5, 10, 15, 20, 30, 35, 36, 37, 38, 39, 40, 50, 100 or more amino acids in length) comprise a domain or motif with at least one activity of the polypeptide according to the invention. Moreover, other biologically active portions in which other regions of the protein are deleted can be prepared by recombinant techniques and evaluated for one or more of the activities described herein. Preferably, the biologically active portions of the polypeptide according to the invention include one or more selected domains/motifs or portions thereof having biological activity.

The term “biological active portion” or “biological activity” means a polypeptide as depicted in SEQ ID NO: 2, 4, 6, 8, 27, or 45, or a homolog thereof or a portion of said polypeptide which still has at least 10% or 20%, preferably 30%, 40%, 50% or 60%, especially preferably 70%, 75%, 80%, 90% or 95% of the enzymatic or biological activity of the natural or starting enzyme or protein.

In the process according to the invention nucleic acid sequences or molecules can be used, which, if appropriate, contain synthetic, non-natural or modified nucleotide bases, which can be incorporated into DNA or RNA. Said synthetic, non-natural or modified bases can for example increase the stability of the nucleic acid molecule outside or inside a cell. The nucleic acid molecules of the invention can contain the same modifications as aforementioned.

As used in the present context the term “nucleic acid molecule” may also encompass the untranslated sequence or molecule located at the 3′ and at the 5′ end of the coding gene region, for example at least 500, preferably 200, especially preferably 100, nucleotides of the sequence upstream of the 5′ end of the coding region and at least 100, preferably 50, especially preferably 20, nucleotides of the sequence downstream of the 3′ end of the coding gene region. It is often advantageous only to choose the coding region for cloning and expression purposes.

Preferably, the nucleic acid molecule used in the process according to the invention or the nucleic acid molecule of the invention is an isolated nucleic acid molecule. In one embodiment, the nucleic acid molecule of the invention is the nucleic acid molecule used in the process of the invention.

In various embodiments, the isolated nucleic acid molecule used in the process according to the invention may, for example comprise less than approximately 5 kb, 4 kb, 3 kb, 2 kb, 1 kb, 0.5 kb or 0.1 kb nucleotide sequences which naturally flank the nucleic acid molecule in the genomic DNA of the cell from which the nucleic acid molecule originates.

The nucleic acid molecules used in the process, for example the polynucleotide of the invention or of a part thereof can be isolated using molecular-biological standard techniques and the sequence information provided herein. Also, for example a homologous sequence or homologous, conserved sequence regions at the DNA or amino acid level can be identified with the aid of comparison algorithms. The former can be used as hybridization probes under standard hybridization techniques (for example those described in Sambrook et al., supra) for isolating further nucleic acid sequences useful in this process.

A nucleic acid molecule encompassing a complete sequence of the nucleic acid molecules used in the process, for example the polynucleotide of the invention, or a part thereof may additionally be isolated by polymerase chain reaction, oligonucleotide primers based on this sequence or on parts thereof being used. For example, a nucleic acid molecule comprising the complete sequence or part thereof can be isolated by polymerase chain reaction using oligonucleotide primers which have been generated on the basis of this very sequence. For example, mRNA can be isolated from cells (for example by means of the guanidinium thiocyanate extraction method of Chirgwin et al., Biochemistry 18, 5294 (1979)) and cDNA can be generated by means of reverse transcriptase (for example Moloney, MLV reverse transcriptase, available from Gibco/BRL, Bethesda, Md., or AMV reverse transcriptase, obtainable from Seikagaku America, Inc., St. Petersburg, Fla.).

Synthetic oligonucleotide primers for the amplification by means of polymerase chain reaction can be generated on the basis of a sequence shown herein, using known methods.

Moreover, it is possible to identify a conserved protein by carrying out protein sequence alignments with the polypeptide encoded by the nucleic acid molecules of the present invention, in particular with the sequences encoded by the nucleic acid molecule shown in SEQ ID NO: 1, 3, 5, 7, 26, or 44, or a homolog thereof, from which conserved regions, and in turn, degenerate primers can be derived. Conserved regions are those, which show a very little variation in the amino acid in one particular position of several homologs from different origin. Moreover, it is possible to identify conserved regions from various organisms by carrying out protein sequence alignments with the polypeptide encoded by the nucleic acid of the present invention, in particular with the sequences of the polypeptide molecule shown in SEQ ID NO: 2, 4, 6, 8, 27, or 45, or a homolog thereof, from which conserved regions, and in turn, degenerate primers can be derived.

Conserved domains can be identified from all sequences and are described using a subset of the standard Prosite notation, e.g. the pattern Y-x(21,23)-[FW] means that a conserved tyrosine is separated by minimum 21 and maximum 23 amino acid residues from either a phenylalanine or tryptophane. Patterns can match at least 80% of the investigated proteins. Conserved patterns can be identified with the software tool MEME version 3.5.1 or manually. MEME is described by Timothy L. Bailey and Charles Elkan (Proceedings of the Second International Conference on Intelligent Systems for Molecular Biology, pp. 28-36, AAAI Press, Menlo Park, Calif., 1994). The source code for the stand-alone program is publicly available from the San Diego Supercomputer centre. The Prosite patterns of the conserved domains can be used to search for protein sequences matching this pattern. Various established Bioinformatic centres provide public internet portals for using those patterns in database searches (e.g. PIR (Protein Information Resource, located at Georgetown University Medical Center) or ExPASy (Expert Protein Analysis System)). Alternatively, stand-alone software is available, like the program Fuzzpro, which is part of the EMBOSS software package. For example, the program Fuzzpro not only allows searching for an exact pattern-protein match but also allows setting various ambiguities in the performed search.

Degenerate primers can then be utilized by PCR for the amplification of fragments of novel proteins having above-mentioned activity, e.g. conferring increased herbicide tolerance or resistance, as compared to a corresponding, e.g. non-transformed, wild type plant cell, plant or part thereof after increasing the expression or activity or having the activity of a protein as shown in SEQ ID NO: 2, 4, 6, 8, 27, or 45, or a homolog thereof or further functional homologs of the polypeptide of the invention from other organisms.

These fragments can then be utilized as hybridization probe for isolating the complete gene sequence. As an alternative, the missing 5′ and 3′ sequences can be isolated by means of RACE-PCR. A nucleic acid molecule according to the invention can be amplified using cDNA or, as an alternative, genomic DNA as template and suitable oligonucleotide primers, following standard PCR amplification techniques. The nucleic acid molecule amplified thus can be cloned into a suitable vector and characterized by means of DNA sequence analysis. Oligonucleotides, which correspond to one of the nucleic acid molecules used in the process can be generated by standard synthesis methods, for example using an automatic DNA synthesizer.

Nucleic acid molecules which are advantageously for the process according to the invention can be isolated based on their homology to the nucleic acid molecules disclosed herein using the sequences or part thereof as or for the generation of a hybridization probe and following standard hybridization techniques under stringent hybridization conditions. In this context, it is possible to use, for example, isolated one or more nucleic acid molecules of at least 15, 20, 25, 30, 35, 40, 50, 60 or more nucleotides, preferably of at least 15, 20 or 25 nucleotides in length which hybridize under stringent conditions with the above-described nucleic acid molecules, in particular with those which encompass a nucleotide sequence of the nucleic acid molecule used in the process of the invention or encoding a protein used in the invention or of the nucleic acid molecule of the invention. Nucleic acid molecules with 30, 50, 100, 250 or more nucleotides may also be used.

By “hybridizing” it is meant that such nucleic acid molecules hybridize under conventional hybridization conditions, preferably under stringent conditions such as described by, e.g., Sambrook (Molecular Cloning; A Laboratory Manual, 2nd Edition, Cold Spring Harbor Laboratory Press, Cold Spring Harbor, N.Y. (1989)) or in Current Protocols in Molecular Biology, John Wiley & Sons, N.Y. (1989), 6.3.1-6.3.6.

According to the invention, DNA as well as RNA molecules of the nucleic acid of the invention can be used as probes. Further, as template for the identification of functional homologues Northern blot assays as well as Southern blot assays can be performed. The Northern blot assay advantageously provides further information about the expressed gene product: e.g. expression pattern, occurrence of processing steps, like splicing and capping, etc. The Southern blot assay provides additional information about the chromosomal localization and organization of the gene encoding the nucleic acid molecule of the invention.

A preferred, non-limiting example of stringent hybridization conditions are hybridizations in 6×sodium chloride/sodium citrate (═SSC) at approximately 45° C., followed by one or more wash steps in 0.2×SSC, 0.1% SDS at 50 to 65° C., for example at 50° C., 55° C. or 60° C. The skilled worker knows that these hybridization conditions differ as a function of the type of the nucleic acid and, for example when organic solvents are present, with regard to the temperature and concentration of the buffer. The temperature under “standard hybridization conditions” differs for example as a function of the type of the nucleic acid between 42° C. and 58° C., preferably between 45° C. and 50° C. in an aqueous buffer with a concentration of 0.1×, 0.5×, 1×, 2×, 3×, 4× or 5×SSC (pH 7.2). If organic solvent(s) is/are present in the abovementioned buffer, for example 50% formamide, the temperature under standard conditions is approximately 40° C., 42° C. or 45° C. The hybridization conditions for DNA:DNA hybrids are preferably for example 0.1×SSC and 20° C., 25° C., 30° C., 35° C., 40° C. or 45° C., preferably between 30° C. and 45° C. The hybridization conditions for DNA:RNA hybrids are preferably for example 0.1×SSC and 30° C., 35° C., 40° C., 45° C., 50° C. or 55° C., preferably between 45° C. and 55° C. The abovementioned hybridization temperatures are determined for example for a nucleic acid approximately 100 bp (=base pairs) in length and a G+C content of 50% in the absence of formamide. The skilled worker knows to determine the hybridization conditions required with the aid of textbooks, for example the ones mentioned above, or from the following textbooks: Sambrook et al., “Molecular Cloning”, Cold Spring Harbor Laboratory, 1989; Hames and Higgins (Ed.) 1985, “Nucleic Acids Hybridization: A Practical Approach”, IRL Press at Oxford University Press, Oxford; Brown (Ed.) 1991, “Essential Molecular Biology: A Practical Approach”, IRL Press at Oxford University Press, Oxford.

A further example of one such stringent hybridization condition is hybridization at 4×SSC at 65° C., followed by a washing in 0.1×SSC at 65° C. for one hour. Alternatively, an exemplary stringent hybridization condition is in 50% formamide, 4×SSC at 42° C. Further, the conditions during the wash step can be selected from the range of conditions delimited by low-stringency conditions (approximately 2×SSC at 50° C.) and high-stringency conditions (approximately 0.2×SSC at 50° C., preferably at 65° C.) (20×SSC: 0.3 M sodium citrate, 3 M NaCl, pH 7.0). In addition, the temperature during the wash step can be raised from low-stringency conditions at room temperature, approximately 22° C., to higher-stringency conditions at approximately 65° C. Both of the parameters salt concentration and temperature can be varied simultaneously, or else one of the two parameters can be kept constant while only the other is varied. Denaturants, for example formamide or SDS, may also be employed during the hybridization. In the presence of 50% formamide, hybridization is preferably effected at 42° C. Relevant factors like 1) length of treatment, 2) salt conditions, 3) detergent conditions, 4) competitor DNAs, 5) temperature and 6) probe selection can be combined case by case so that not all possibilities can be mentioned herein.

Thus, in a preferred embodiment, Northern blots are prehybridized with Rothi-Hybri-Quick buffer (Roth, Karlsruhe) at 68° C. for 2 h. Hybridization with radioactive labelled probe is done overnight at 68° C. Subsequent washing steps are performed at 68° C. with 1×SSC. For Southern blot assays the membrane is prehybridized with Rothi-Hybri-Quick buffer (Roth, Karlsruhe) at 68° C. for 2 h. The hybridization with radioactive labelled probe is conducted over night at 68° C. Subsequently the hybridization buffer is discarded and the filter shortly washed using 2×SSC; 0,1% SDS. After discarding the washing buffer new 2×SSC; 0,1% SDS buffer is added and incubated at 68° C. for 15 minutes. This washing step is performed twice followed by an additional washing step using 1×SSC; 0,1% SDS at 68° C. for 10 min.

Some examples of conditions for DNA hybridization (Southern blot assays) and wash step are shown herein below:

(1) Hybridization conditions can be selected, for example, from the following conditions:
(a) 4×SSC at 65° C.,
(b) 6×SSC at 45° C.,
(c) 6×SSC, 100 mg/ml denatured fragmented fish sperm DNA at 68° C.,
(d) 6×SSC, 0.5% SDS, 100 mg/ml denatured salmon sperm DNA at 68° C.,
(e) 6×SSC, 0.5% SDS, 100 mg/ml denatured fragmented salmon sperm DNA, 50% formamide at 42° C.,
(f) 50% formamide, 4×SSC at 42° C.,
(g) 50% (v/v) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C.,
(h) 2× or 4×SSC at 50° C. (low-stringency condition), or
(i) 30 to 40% formamide, 2× or 4×SSC at 42° C. (low-stringency condition).
   (2) Wash steps can be selected, for example, from the following conditions:
(a) 0.015 M NaCl/0.0015 M sodium citrate/0.1% SDS at 50° C.
(b) 0.1×SSC at 65° C.
(c) 0.1×SSC, 0.5% SDS at 68° C.
(d) 0.1×SSC, 0.5% SDS, 50% formamide at 42° C.
(e) 0.2×SSC, 0.1% SDS at 42° C.
(f) 2×SSC at 65° C. (low-stringency condition).

Polypeptides having above-mentioned activity, i.e. conferring increased herbicide tolerance or resistance, as compared to a corresponding, e.g. non-transformed, wild type plant cell, plant or part thereof, derived from other organisms, can be encoded by other DNA sequences which hybridize to the sequences shown in SEQ ID NO: 1, 3, 5, 7, 26, or 44, or a homolog thereof, under relaxed hybridization conditions and which code on expression for peptides conferring the increased herbicide tolerance or resistance, as compared to a corresponding, e.g. non-transformed, wild type plant cell, plant or part thereof.

Further, some applications have to be performed at low stringency hybridization conditions, without any consequences for the specificity of the hybridization. For example, a Southern blot analysis of total DNA could be probed with a nucleic acid molecule of the present invention and washed at low stringency (55° C. in 2×SSPE, 0,1% SDS). The hybridization analysis could reveal a simple pattern of only genes encoding polypeptides of the present invention or used in the process of the invention, e.g. having the herein-mentioned activity of enhancing the increased herbicide tolerance or resistance, as compared to a corresponding, e.g. non-transformed, wild type plant cell, plant or part thereof. A further example of such low-stringent hybridization conditions is 4×SSC at 50° C. or hybridization with 30 to 40% formamide at 42° C. Such molecules comprise those which are fragments, analogues or derivatives of the polypeptide of the invention or used in the process of the invention and differ, for example, by way of amino acid and/or nucleotide deletion(s), insertion(s), substitution (s), addition(s) and/or recombination (s) or any other modification(s) known in the art either alone or in combination from the above-described amino acid sequences or their underlying nucleotide sequence(s). However, it is preferred to use high stringency hybridization conditions.

Hybridization should advantageously be carried out with fragments of at least 5, 10, 15, 20, 25, 30, 35 or 40 bp, advantageously at least 50, 60, 70 or 80 bp, preferably at least 90, 100 or 110 bp. Most preferably are fragments of at least 15, 20, 25 or 30 bp. Preferably are also hybridizations with at least 100 bp or 200, very especially preferably at least 400 bp in length. In an especially preferred embodiment, the hybridization should be carried out with the entire nucleic acid sequence with conditions described above.

The terms “fragment”, “fragment of a sequence” or “part of a sequence” mean a truncated sequence of the original sequence referred to. The truncated sequence (nucleic acid or protein sequence) can vary widely in length; the minimum size being a sequence of sufficient size to provide a sequence with at least a comparable function and/or activity of the original sequence or molecule referred to or hybridizing with the nucleic acid molecule of the invention or used in the process of the invention under stringent conditions, while the maximum size is not critical. In some applications, the maximum size usually is not substantially greater than that required to provide the desired activity and/or function(s) of the original sequence.

Typically, the truncated amino acid sequence or molecule will range from about 5 to about 310 amino acids in length. More typically, however, the sequence will be a maximum of about 250 amino acids in length, preferably a maximum of about 200 or 100 amino acids. It is usually desirable to select sequences of at least about 10, 12 or 15 amino acids, up to a maximum of about 20 or 25 amino acids.

The term “epitope” relates to specific immunoreactive sites within an antigen, also known as antigenic determinates. These epitopes can be a linear array of monomers in a polymeric composition—such as amino acids in a protein—or consist of or comprise a more complex secondary or tertiary structure. Those of skill will recognize that immunogens (i.e., substances capable of eliciting an immune response) are antigens; however, some antigen, such as haptens, are not immunogens but may be made immunogenic by coupling to a carrier molecule. The term “antigen” includes references to a substance to which an antibody can be generated and/or to which the antibody is specifically immunoreactive.

In one embodiment the present invention relates to a epitope of the polypeptide of the present invention or used in the process of the present invention and confers an increased herbicide tolerance or resistance as compared to a corresponding, e.g. non-transformed, wild type plant cell, plant or part thereof.

The term “one or several amino acids” relates to at least one amino acid but not more than that number of amino acids, which would result in a homology of below 50% identity. Preferably, the identity is more than 70% or 80%, more preferred are 85%, 90%, 91%, 92%, 93%, 94% or 95%, even more preferred are 96%, 97%, 98%, or 99% identity.

Further, the nucleic acid molecule of the invention comprises a nucleic acid molecule, which is a complement of one of the nucleotide sequences of above mentioned nucleic acid molecules or a portion thereof. A nucleic acid molecule or its sequence which is complementary to one of the nucleotide molecules or sequences shown in SEQ ID NO: 1, 3, 5, 7, 26, or 44, or a homolog thereof, is one which is sufficiently complementary to one of the nucleotide molecules or sequences shown in SEQ ID NO: 1, 3, 5, 7, 26, or 44, or a homolog thereof such that it can hybridize to one of the nucleotide sequences shown in SEQ ID NO: 1, 3, 5, 7, 26, or 44, or a homolog thereof, thereby forming a stable duplex. Preferably, the hybridization is performed under stringent hybridization conditions. However, a complement of one of the herein disclosed sequences is preferably a sequence complement thereto according to the base pairing of nucleic acid molecules well known to the skilled person. For example, the bases A and G undergo base pairing with the bases T and U or C, resp. and visa versa. Modifications of the bases can influence the base-pairing partner.

The nucleic acid molecule of the invention comprises a nucleotide sequence which is at least about 30%, 35%, 40% or 45%, preferably at least about 50%, 55%, 60% or 65%, more preferably at least about 70%, 80%, or 90%, and even more preferably at least about 95%, 97%, 98%, 99% or more homologous to a nucleotide sequence shown in SEQ ID NO: 1, 3, 5, 7, 26, or 44, or a homolog thereof, or a portion thereof and preferably has above mentioned activity, in particular having a herbicide tolerance or resistance increasing activity after increasing the activity or an activity of a gene as shown in SEQ ID NO: 1, 3, 5, 7, 26, or 44, or a homolog thereof or of a gene product, by for example expression either in the cytosol or cytoplasm or in an organelle such as a plastid or mitochondria or both, preferably in plastids.

In one embodiment, the nucleic acid molecules comprising the sequence of SEQ ID NO: 1, 3, 5, 7, 26, or 44, or a homolog thereof or gene products encoded by said nucleic acid molecules are expressed in combination with a targeting signal as described herein.

The nucleic acid molecule of the invention comprises a nucleotide sequence or molecule which hybridizes, preferably hybridizes under stringent conditions as defined herein, to one of the nucleotide sequences or molecule shown in SEQ ID NO: 1, 3, 5, 7, 26, or 44, or a homolog thereof, or a portion thereof and encodes a protein having above-mentioned activity, e.g. conferring an increased herbicide tolerance or resistance, as compared to a corresponding, e.g. non-transformed, wild type plant cell, plant or part thereof by for example expression either in the cytosol or in an organelle such as a plastid or mitochondria or both, preferably in plastids, and optionally, having the activity of an Alopecurus CytP450 enzyme.

Moreover, the nucleic acid molecule of the invention can comprise only a portion of the coding region of one of the sequences shown in SEQ ID NO: 1, 3, 5, 7, 26, or 44, or a homolog thereof, for example a fragment which can be used as a probe or primer or a fragment encoding a biologically active portion of the polypeptide of the present invention or of a polypeptide used in the process of the present invention, i.e. having above-mentioned activity, e.g. conferring an increased herbicide tolerance or resistance, as compared to a corresponding, e.g. non-transformed, wild type plant cell, plant or part thereof f its activity is increased by for example expression either in the cytosol or in an organelle such as a plastid or mitochondria or both, preferably in plastids. The nucleotide sequences determined from the cloning of the present protein-according-to-the-invention-encoding gene allows for the generation of probes and primers designed for use in identifying and/or cloning its homologues in other cell types and organisms. The probe/primer typically comprises substantially purified oligonucleotide. The oligonucleotide typically comprises a region of nucleotide sequence that hybridizes under stringent conditions to at least about 12, 15 preferably about 20 or 25, more preferably about 40, 50 or 75 consecutive nucleotides of a sense strand of one of the sequences set forth, e.g., in SEQ ID NO: 1, 3, 5, 7, 26, or 44, or a homolog thereof, an anti-sense sequence of one of the sequences, e.g., set forth in SEQ ID NO: 1, 3, 5, 7, 26, or 44, or a homolog thereof, or naturally occurring mutants thereof. Primers based on a nucleotide of invention can be used in PCR reactions to clone homologues of the polypeptide of the invention or of the polypeptide used in the process of the invention, e.g. as the primers described in the examples of the present invention, e.g. as shown in the examples. A PCR with primers based on SEQ ID NO: 1, 3, 5, 7, 26, or 44 will result in a fragment of the gene product as shown SEQ ID NO: 2, 4, 6, 8, 27, or 45, or a homolog thereof.

Primer sets are interchangeable. The person skilled in the art knows to combine said primers to result in the desired product, e.g. in a full length clone or a partial sequence. Probes based on the sequences of the nucleic acid molecule of the invention or used in the process of the present invention can be used to detect transcripts or genomic sequences encoding the same or homologous proteins. The probe can further comprise a label group attached thereto, e.g. the label group can be a radioisotope, a fluorescent compound, an enzyme, or an enzyme co-factor. Such probes can be used as a part of a genomic marker test kit for identifying cells which express an polypeptide of the invention or used in the process of the present invention, such as by measuring a level of an encoding nucleic acid molecule in a sample of cells, e.g., detecting mRNA levels or determining, whether a genomic gene comprising the sequence of the polynucleotide of the invention or used in the processes of the present invention has been mutated or deleted.

The nucleic acid molecule of the invention encodes a polypeptide or portion thereof which includes an amino acid sequence which is sufficiently homologous to the amino acid sequence shown in SEQ ID NO: 2, 4, 6, 8, 27, or 45, or a homolog thereof such that the protein or portion thereof maintains the ability to participate in increasing herbicide tolerance or resistance, as compared to a corresponding, e.g. non-transformed, wild type plant cell, plant or part thereof, in particular increasing the activity as mentioned above or as described in the examples in plants is comprised.

As used herein, the language “sufficiently homologous” refers to proteins or portions thereof which have amino acid sequences which include a minimum number of identical or equivalent amino acid residues (e.g., an amino acid residue which has a similar side chain as an amino acid residue in one of the sequences of the polypeptide of the present invention) to an amino acid sequence shown in SEQ ID NO: 2, 4, 6, 8, 27, or 45, or a homolog thereof such that the protein or portion thereof is able to participate in increasing herbicide tolerance or resistance, as compared to a corresponding, e.g. non-transformed, wild type plant cell, plant or part thereof.

In one embodiment, the nucleic acid molecule of the present invention comprises a nucleic acid that encodes a portion of the protein of the present invention. The protein is at least about 30%, 35%, 40%, 45% or 50%, preferably at least about 55%, 60%, 65% or 70%, and more preferably at least about 75%, 80%, 85%, 90%, 91%, 92%, 93% or 94% and most preferably at least about 95%, 97%, 98%, 99% or more homologous to an entire amino acid sequence SEQ ID NO: 2, 4, 6, 8, 27, or 45, and having above-mentioned activity, e.g. conferring an increased herbicide tolerance or resistance, as compared to a corresponding, e.g. non-transformed, wild type plant cell, plant or part thereof by for example expression either in the cytosol or in an organelle such as a plastid or mitochondria or both, preferably in plastids. In a preferred embodiment, such homologs refer to proteins comprising the sequences of SEQ ID NO: 9, 10, 11, 12, 13, 14, 15, 16, 17, 18, 19, 20, 21, 22, 23, 24, 25, 28, 29, 30, 31, 32, 33, 34, 35, 36, 37, 38, 39, 40, 41, 42, 43, 46, 47, 48, 49, 50, 51, 52, 53, 54, 55, 56, 57, 58, 59, 60, 61, 62, 63, 64, 65, 66, 67, 68, or 69.

Portions of proteins encoded by the nucleic acid molecule of the invention are preferably biologically active, preferably having above-mentioned annotated activity, e.g. conferring an increased herbicide tolerance or resistance, as compared to a corresponding, e.g. non-transformed, wild type plant cell, plant or part thereof after increase of activity.

As mentioned herein, the term “biologically active portion” is intended to include a portion, e.g., a domain/motif, that confers an increased herbicide tolerance or resistance, e.g. an increased herbicide tolerance or resistance-related trait, as compared to a corresponding, e.g. non-transformed, wild type plant cell, plant or part thereof or has an immunological activity such that it is binds to an antibody binding specifically to the polypeptide of the present invention or a polypeptide used in the process of the present invention for increasing herbicide tolerance or resistance, as compared to a corresponding, e.g. non-transformed, wild type plant cell, plant or part thereof.

The invention further relates to nucleic acid molecules that differ from one of the nucleotide sequences shown in SEQ ID NO: 1, 3, 5, 7, 26, or 44, or a homolog thereof (and portions thereof) due to degeneracy of the genetic code and thus encode a polypeptide of the present invention, in particular a polypeptide having above mentioned activity, e.g. as that polypeptides depicted by the sequence shown in SEQ ID NO: 2, 4, 6, 8, 27, or 45, or the functional homologues. Advantageously, the nucleic acid molecule of the invention comprises, or in an other embodiment has, a nucleotide sequence encoding a protein comprising, or in an other embodiment having, an amino acid sequence shown in SEQ ID NO: 2, 4, 6, 8, 27, or 45, or the functional homologues. In a still further embodiment, the nucleic acid molecule of the invention encodes a full length protein which is substantially homologous to an amino acid sequence shown in SEQ ID NO: 2, 4, 6, 8, 27, or 45, or the functional homologues.

In addition, it will be appreciated by those skilled in the art that DNA sequence polymorphisms that lead to changes in the amino acid sequences may exist within a population. Such genetic polymorphism in the gene encoding the polypeptide of the invention or comprising the nucleic acid molecule of the invention may exist among individuals within a population due to natural variation.

Nucleic acid molecules corresponding to natural variants homologues of a nucleic acid molecule of the invention, which can also be a cDNA, can be isolated based on their homology to the nucleic acid molecules disclosed herein using the nucleic acid molecule of the invention, or a portion thereof, as a hybridization probe according to standard hybridization techniques under stringent hybridization conditions.

Accordingly, in another embodiment, a nucleic acid molecule of the invention is at least 15, 20, 25 or 30 nucleotides in length. Preferably, it hybridizes under stringent conditions to a nucleic acid molecule comprising a nucleotide sequence of the nucleic acid molecule of the present invention or used in the process of the present invention, e.g. comprising the sequence shown in SEQ ID NO: 1, 3, 5, 7, 26, or 44, or a homolog thereof. The nucleic acid molecule is preferably at least 20, 30, 50, 100, 250 or more nucleotides in length.

The term “hybridizes under stringent conditions” is defined above. In one embodiment, the term “hybridizes under stringent conditions” is intended to describe conditions for hybridization and washing under which nucleotide sequences at least 30%, 40%, 50% or 65% identical to each other typically remain hybridized to each other. Preferably, the conditions are such that sequences at least about 70%, more preferably at least about 75% or 80%, and even more preferably at least about 85%, 90% or 95% or more identical to each other typically remain hybridized to each other.

Preferably, nucleic acid molecule of the invention that hybridizes under stringent conditions to a sequence shown in SEQ ID NO: 1, 3, 5, 7, 26, or 44, or a homolog thereof corresponds to a naturally-occurring nucleic acid molecule of the invention. As used herein, a “naturally-occurring”nucleic acid molecule refers to an RNA or DNA molecule having a nucleotide sequence that occurs in nature (e.g., encodes a natural protein). Preferably, the nucleic acid molecule encodes a natural protein having above-mentioned activity, e.g. conferring increasing herbicide tolerance or resistance, after increasing the expression or activity thereof or the activity of a protein of the invention or used in the process of the invention by for example expression the nucleic acid sequence of the gene product in the cytosol and/or in an organelle such as a plastid or mitochondria, preferably in plastids.

In addition to naturally-occurring variants of the sequences of the polypeptide or nucleic acid molecule of the invention as well as of the polypeptide or nucleic acid molecule used in the process of the invention that may exist in the population, the skilled artisan will further appreciate that changes can be introduced by mutation into a nucleotide sequence of the nucleic acid molecule encoding the polypeptide of the invention or used in the process of the present invention, thereby leading to changes in the amino acid sequence of the encoded said polypeptide, without altering the functional ability of the polypeptide, preferably not decreasing said activity.

For example, nucleotide substitutions leading to amino acid substitutions at “non-essential” amino acid residues can be made in a sequence of the nucleic acid molecule of the invention or used in the process of the invention, e.g. shown in SEQ ID NO: 1, 3, 5, 7, 26, or 44, or a homolog thereof.

A “non-essential” amino acid residue is a residue that can be altered from the wild-type sequence of one without altering the activity of said polypeptide, whereas an “essential” amino acid residue is required for an activity as mentioned above, e.g. leading to increasing herbicide tolerance or resistance, as compared to a corresponding, e.g. non-transformed, wild type plant cell, plant or part thereof in an organism after an increase of activity of the polypeptide. Other amino acid residues, however, (e.g., those that are not conserved or only semi-conserved in the domain having said activity) may not be essential for activity and thus are likely to be amenable to alteration without altering said activity.

Further, a person skilled in the art knows that the codon usage between organisms can differ. Therefore, he may adapt the codon usage in the nucleic acid molecule of the present invention to the usage of the organism or the cell compartment for example of the plastid or mitochondria in which the polynucleotide or polypeptide is expressed. In a particular preferred embodiment, codon-adapted nucleic acid molecules of the present invention comprise the sequence of SEQ ID NO: 70, 71, 72, 73, or 74, which represent codon-adapted nucleic acid molecules corresponding to SEQ ID NO: 1, 3, 5, 24, or 42, respectively.

Accordingly, the invention relates to nucleic acid molecules encoding a polypeptide having above-mentioned activity, in an organism or parts thereof by for example expression either in the cytosol or in an organelle such as a plastid or mitochondria or both, preferably in plastids that contain changes in amino acid residues that are not essential for said activity. Such polypeptides differ in amino acid sequence from a sequence contained in the sequences shown in SEQ ID NO: 2, 4, 6, 8, 27, or 45, or a homolog thereof yet retain said activity described herein. The nucleic acid molecule can comprise a nucleotide sequence encoding a polypeptide, wherein the polypeptide comprises an amino acid sequence at least about 50% identical to an amino acid sequence shown SEQ ID NO: 2, 4, 6, 8, 27, or 45, or a homolog thereof and is capable of participation in increasing herbicide tolerance or resistance, as compared to a corresponding, e.g. non-transformed, wild type plant cell, plant or part thereof after increasing its activity, e.g. its expression by for example expression either in the cytosol or in an organelle such as a plastid or mitochondria or both, preferably in plastids. Preferably, the protein encoded by the nucleic acid molecule is at least about 60% identical to the sequence shown in SEQ ID NO: 2, 4, 6, 8, 27, or 45, or a homolog thereof, more preferably at least about 70% identical to one of the sequences shown in SEQ ID NO: 2, 4, 6, 8, 27, or 45, or a homolog thereof, even more preferably at least about 80%, 90%, 95% homologous to the sequence shown in SEQ ID NO: 2, 4, 6, 8, 27, or 45, or a homolog thereof, and most preferably at least about 96%, 97%, 98%, or 99% identical to the sequence shown in SEQ ID NO: 2, 4, 6, 8, 27, or 45, or a homolog thereof.

To determine the percentage homology (=identity, herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology” as used in the present context corresponds to amino acid or nucleic acid “identity”. The percentage homology between the two sequences is a function of the number of identical positions shared by the sequences (i.e. % homology=number of identical positions/total number of positions×100). The terms “homology” and “identity” are thus to be considered as synonyms.

For the determination of the percentage homology (=identity) of two or more amino acids or of two or more nucleotide sequences several computer software programs have been developed. The homology of two or more sequences can be calculated with for example the software fasta, which presently has been used in the version fasta 3 (W. R. Pearson and D. J. Lipman, PNAS 85, 2444 (1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of homologies of different sequences is the standard blast program, which is included in the Biomax pedant software (Biomax, Munich, Federal Republic of Germany). This leads unfortunately sometimes to suboptimal results since blast does not always include complete sequences of the subject and the querry. Nevertheless as this program is very efficient it can be used for the comparison of a huge number of sequences. The following settings are typically used for such a comparisons of sequences: -p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mismatch (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algorithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution, 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the European Molecular Biology Open Software Suite (EMBOSS) (Trends in Genetics 16 (6), 276 (2000)). Therefore preferably the calculations to determine the percentages of sequence homology are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which has 80% homology with sequence SEQ ID NO: 1 at the nucleic acid level is understood as meaning a sequence which, upon comparison with the sequence SEQ ID NO: 1 by the above program “Needle” with the above parameter set, has a 80% homology.

Homology between two polypeptides is understood as meaning the identity of the amino acid sequence over in each case the entire sequence length which is calculated by comparison with the aid of the above program “Needle” using Matrix: EBLOSUM62, Gap_penalty: 8.0, Extend_penalty: 2.0.

For example a sequence which has a 80% homology with sequence SEQ ID NO: 2 at the protein level is understood as meaning a sequence which, upon comparison with the sequence SEQ ID NO: 2 by the above program “Needle” with the above parameter set, has a 80% homology.

Functional equivalents derived from the nucleic acid sequence as shown in SEQ ID NO: 1, 3, 5, 7, 26, or 44, or a homolog thereof according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in SEQ ID NO: 2, 4, 6, 8, 27, or 45, or a homolog thereof according to the invention and encode polypeptides having essentially the same properties as the polypeptide as shown in SEQ ID NO: 2, 4, 6, 8, 27, or 45, or a homolog thereof.

Functional equivalents derived from one of the polypeptides as shown in SEQ ID NO: 2, 4, 6, 8, 27, or 45, or a homolog thereof according to the invention by substitution, insertion or deletion have at least 30%, 35%, 40%, 45% or 50%, preferably at least 55%, 60%, 65% or 70% by preference at least 80%, especially preferably at least 85% or 90%, 91%, 92%, 93% or 94%, very especially preferably at least 95%, 97%, 98% or 99% homology with one of the polypeptides as shown in SEQ ID NO: 2, 4, 6, 8, 27, or 45, or a homolog thereof according to the invention and having essentially the same properties as the polypeptide as shown in SEQ ID NO: 2, 4, 6, 8, 27, or 45, or a homolog thereof.

“Essentially the same properties” of a functional equivalent is above all understood as meaning that the functional equivalent has above mentioned activity, by for example expression either in the cytosol or in an organelle such as a plastid or mitochondria or both, preferably in plastids while increasing the amount of protein, activity or function of said functional equivalent in an organism, e.g. a microorgansim, a plant or plant tissue or animal tissue, plant or animal cells or a part of the same.

A nucleic acid molecule encoding an homologous to a protein sequence of SEQ ID NO: 2, 4, 6, 8, 27, or 45, or a homolog thereof can be created by introducing one or more nucleotide substitutions, additions or deletions into a nucleotide sequence of the nucleic acid molecule of the present invention, in particular of SEQ ID NO: 1, 3, 5, 7, 26, or 44, or a homolog thereof such that one or more amino acid substitutions, additions or deletions are introduced into the encoded protein. Mutations can be introduced into the encoding sequences of SEQ ID NO: 1, 3, 5, 7, 26, or 44, or a homolog thereof by standard techniques, such as site-directed mutagenesis and PCR-mediated mutagenesis.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophane), beta-branched side chains (e.g., threonine, valine, isoleucine) and aromatic side chains (e.g., tyrosine, phenylalanine, tryptophane, histidine).

Thus, a predicted nonessential amino acid residue in a polypeptide of the invention or a polypeptide used in the process of the invention is preferably replaced with another amino acid residue from the same family. Alternatively, in another embodiment, mutations can be introduced randomly along all or part of a coding sequence of a nucleic acid molecule of the invention or used in the process of the invention, such as by saturation mutagenesis, and the resultant mutants can be screened for activity described herein to identify mutants that retain or even have increased above mentioned activity, e.g. conferring increased herbicide tolerance or resistance, as compared to a corresponding, e.g. non-transformed, wild type plant cell, plant or part thereof.

Following mutagenesis of one of the sequences as shown herein, the encoded protein can be expressed recombinantly and the activity of the protein can be determined using, for example, assays described herein.

The highest homology of the nucleic acid molecule used in the process according to the invention was found for the following database entries by Gap search.

Homologues of the nucleic acid sequences used, with the sequence shown in SEQ ID NO: 1, 3, 5, 7, 26, or 44, or a homolog thereof, comprise also allelic variants with at least approximately 30%, 35%, 40% or 45% homology, by preference at least approximately 50%, 60% or 70%, more preferably at least approximately 90%, 91%, 92%, 93%, 94% or 95% and even more preferably at least approximately 96%, 97%, 98%, 99% or more homology with one of the nucleotide sequences shown or the abovementioned derived nucleic acid sequences or their homologues, derivatives or analogues or parts of these. Allelic variants encompass in particular functional variants which can be obtained by deletion, insertion or substitution of nucleotides from the sequences shown, preferably from SEQ ID NO: 1, 3, 5, 7, 26, or 44, or a homolog thereof, or from the derived nucleic acid sequences, the intention being, however, that the enzyme activity or the biological activity of the resulting proteins synthesized is advantageously retained or increased.

In one embodiment of the present invention, the nucleic acid molecule of the invention or used in the process of the invention comprises the sequences shown in any of the SEQ ID NO: 1, 3, 5, 7, 26, or 44, or a homolog thereof. It is preferred that the nucleic acid molecule comprises as little as possible other nucleotides not shown in any one of SEQ ID NO: 1, 3, 5, 7, 26, or 44, or a homolog thereof. In one embodiment, the nucleic acid molecule comprises less than 500, 400, 300, 200, 100, 90, 80, 70, 60, 50 or 40 further nucleotides. In a further embodiment, the nucleic acid molecule comprises less than 30, 20 or 10 further nucleotides. In one embodiment, the nucleic acid molecule use in the process of the invention is identical to the sequences shown in SEQ ID NO: 1, 3, 5, 7, 26, or 44, or a homolog thereof.

Also preferred is that the nucleic acid molecule used in the process of the invention encodes a polypeptide comprising the sequence shown in SEQ ID NO: 2, 4, 6, 8, 27, or 45, or a homolog thereof. In one embodiment, the nucleic acid molecule encodes less than 150, 130, 100, 80, 60, 50, 40 or 30 further amino acids. In a further embodiment, the encoded polypeptide comprises less than 20, 15, 10, 9, 8, 7, 6 or 5 further amino acids. In one embodiment used in the inventive process, the encoded polypeptide is identical to the sequences shown in SEQ ID NO: 2, 4, 6, 8, 27, or 45, or a homolog thereof.

In one embodiment, the nucleic acid molecule of the invention or used in the process encodes a polypeptide comprising the sequence shown in SEQ ID NO: 2, 4, 6, 8, 27, or 45, or a homolog thereof comprises less than 100 further nucleotides. In a further embodiment, said nucleic acid molecule comprises less than 30 further nucleotides. In one embodiment, the nucleic acid molecule used in the process is identical to a coding sequence of the sequences shown in SEQ ID NO: 1, 3, 5, 7, 26, or 44, or a homolog thereof.

Polypeptides (=proteins), which still have the essential biological or enzymatic activity of the polypeptide of the present invention conferring increased herbicide tolerance or resistance, as compared to a corresponding, e.g. non-transformed, wild type plant cell, plant or part thereof i.e. whose activity is essentially not reduced, are polypeptides with at least 10% or 20%, by preference 30% or 40%, especially preferably 50% or 60%, very especially preferably 80% or 90 or more of the wild type biological activity or enzyme activity, advantageously, the activity is essentially not reduced in comparison with the activity of a polypeptide shown in SEQ ID NO: 2, 4, 6, 8, 27, or 45, or a homolog thereof expressed under identical conditions.

Homologues of SEQ ID NO: 1, 3, 5, 7, 26, or 44, or of the derived sequences of SEQ ID NO: 2, 4, 6, 8, 27, or 45, or a homolog thereof also mean truncated sequences, cDNA, single-stranded DNA or RNA of the coding and noncoding DNA sequence. Homologues of said sequences are also understood as meaning derivatives, which comprise noncoding regions such as, for example, UTRs, terminators, enhancers or promoter variants. The promoters upstream of the nucleotide sequences stated can be modified by one or more nucleotide substitution(s), insertion(s) and/or deletion(s) without, however, interfering with the functionality or activity either of the promoters, the open reading frame (=ORF) or with the 3′-regulatory region such as terminators or other 3′-regulatory regions, which are far away from the ORF. It is furthermore possible that the activity of the promoters is increased by modification of their sequence, or that they are replaced completely by more active promoters, even promoters from heterologous organisms. Appropriate promoters are known to the person skilled in the art and are mentioned herein below.

In addition to the nucleic acid molecules encoding the polypeptide according to the invention described above, another aspect of the invention pertains to negative regulators of the activity of a nucleic acid molecule comprising the sequence of SEQ ID NO: 1, 3, 5, 7, 26, or 44, or a homolog thereof. Antisense polynucleotides thereto are thought to inhibit the down-regulating activity of those negative regulators by specifically binding the target polynucleotide and interfering with transcription, splicing, transport, translation, and/or stability of the target polynucleotide. Methods are described in the prior art for targeting the antisense polynucleotide to the chromosomal DNA, to a primary RNA transcript, or to a processed mRNA. Preferably, the target regions include splice sites, translation initiation codons, translation termination codons, and other sequences within the open reading frame.

The term “antisense,” for the purposes of the invention, refers to a nucleic acid comprising a polynucleotide that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. It is understood that two polynucleotides may hybridize to each other even if they are not completely complementary to each other, provided that each has at least one region that is substantially complementary to the other. The term “antisense nucleic acid” includes single stranded RNA as well as double-stranded DNA expression cassettes that can be transcribed to produce an antisense RNA. “Active” antisense nucleic acids are anti-sense RNA molecules that are capable of selectively hybridizing with a negative regulator of the activity of a nucleic acid molecules encoding a polypeptide having at least 80% sequence identity with the polypeptide selected from the group according to SEQ ID NO: 2, 4, 6, 8, 27, or 45, or a homolog thereof.

The antisense nucleic acid can be complementary to an entire negative regulator strand, or to only a portion thereof. In an embodiment, the antisense nucleic acid molecule is anti-sense to a “noncoding region” of the coding strand of a nucleotide sequence encoding the polypeptide according to the invention. The term “noncoding region” refers to 5′ and 3′ sequences that flank the coding region that are not translated into amino acids (i.e., also referred to as 5′ and 3′ untranslated regions). The antisense nucleic acid molecule can be complementary to only a portion of the noncoding region of a mRNA. For example, the anti-sense oligonucleotide can be complementary to the region surrounding the translation start site of the mRNA. An antisense oligonucleotide can be, for example, about 5, 10, 15, 20, 25, 30, 35, 40, 45 or 50 nucleotides in length. Typically, the antisense molecules of the present invention comprise an RNA having 60-100% sequence identity with at least 14 consecutive nucleotides of a noncoding region of one of the nucleic acid of SEQ ID NO: 1, 3, 5, 7, 26, or 44, or a homolog thereof. Preferably, the sequence identity will be at least 70%, more preferably at least 75%, 80%, 85%, 90%, 95%, 98% and most preferably 99%.

An antisense nucleic acid of the invention can be constructed using chemical synthesis and enzymatic ligation reactions using procedures known in the art. For example, an anti-sense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl)-uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 5-methyl-2-thiouracil, 3-(3-amino-3-N-2-carboxypropyl)-uracil, acp3 and 2,6-diaminopurine. Alternatively, the antisense nucleic acid can be produced biologically using an expression vector into which a nucleic acid has been subcloned in an anti-sense orientation (i.e., RNA transcribed from the inserted nucleic acid will be of an antisense orientation to a target nucleic acid of interest, described further in the following subsection).

In yet another embodiment, the antisense nucleic acid molecule of the invention is an alpha-anomeric nucleic acid molecule. An alpha-anomeric nucleic acid molecule forms specific double-stranded hybrids with complementary RNA in which, contrary to the usual b-units, the strands run parallel to each other (Gaultier et al., Nucleic Acids. Res. 15, 6625 (1987)). The antisense nucleic acid molecule can also comprise a 2′-o-methylribonucleotide (Inoue et al., Nucleic Acids Res. 15, 6131 (1987)) or a chimeric RNA-DNA analogue (Inoue et al., FEBS Lett. 215, 327 (1987)).

The antisense nucleic acid molecules of the invention are typically administered to a cell or generated in situ such that they hybridize with or bind to cellular mRNA and/or genomic DNA. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The anti-sense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic (including plant) promoter are preferred.

As an alternative to antisense polynucleotides, ribozymes, sense polynucleotides, or double stranded RNA (dsRNA) can be used to reduce expression of the polypeptide according to the invention polypeptide. By “ribozyme” is meant a catalytic RNA-based enzyme with ribonuclease activity which is capable of cleaving a single-stranded nucleic acid, such as an mRNA, to which it has a complementary region. Ribozymes (e.g., hammerhead ribozymes described in Haselhoff and Gerlach, Nature 334, 585 (1988)) can be used to catalytically cleave the mRNA transcripts to thereby inhibit translation of the mRNA. A ribozyme having specificity for the polypeptide according to the invention-encoding nucleic acid can be designed based upon the nucleotide sequence of the polypeptide according to the invention cDNA, as disclosed herein or on the basis of a heterologous sequence to be isolated according to methods taught in this invention. For example, a derivative of a Tetrahymena L-19 IVS RNA can be constructed in which the nucleotide sequence of the active site is complementary to the nucleotide sequence to be cleaved in the polypeptide according to the invention-encoding mRNA. See, e.g. U.S. Pat. Nos. 4,987,071 and 5,116,742 to Cech et al. alternatively, the mRNA can be used to select a catalytic RNA having a specific ribonuclease activity from a pool of RNA molecules. See, e.g. Bartel D., and Szostak J. W., Science 261, 1411 (1993). In preferred embodiments, the ribozyme will contain a portion having at least 7, 8, 9, 10, 12, 14, 16, 18 or 20 nucleotides, and more preferably 7 or 8 nucleotides, that have 100% complementarity to a portion of the target RNA. Methods for making ribozymes are known to those skilled in the art. See, e.g. U.S. Pat. Nos. 6,025,167, 5,773,260 and 5,496,698.

The term “dsRNA,” as used herein, refers to RNA hybrids comprising two strands of RNA. The dsRNAs can be linear or circular in structure. In a preferred embodiment, dsRNA is specific for a polynucleotide encoding either the polypeptide according to SEQ ID NO: 2, 4, 6, 8, 27, or 45, or a homolog thereof or a polypeptide having at least 70% sequence identity with a polypeptide according to SEQ ID NO: 2, 4, 6, 8, 27, or 45, or a homolog thereof. The hybridizing RNAs may be substantially or completely complementary. By “substantially complementary,” is meant that when the two hybridizing RNAs are optimally aligned using the BLAST program as described above, the hybridizing portions are at least 95% complementary. Preferably, the dsRNA will be at least 100 base pairs in length. Typically, the hybridizing RNAs will be of identical length with no over hanging 5′ or 3′ ends and no gaps. However, dsRNAs having 5′ or 3′ overhangs of up to 100 nucleotides may be used in the methods of the invention.

The dsRNA may comprise ribonucleotides or ribonucleotide analogs, such as 2′-O-methyl ribosyl residues, or combinations thereof. See, e.g. U.S. Pat. Nos. 4,130,641 and 4,024,222. A dsRNA polyriboinosinic acid: polyribocytidylic acid is described in U.S. Pat. No. 4,283,393. Methods for making and using dsRNA are known in the art. One method comprises the simultaneous transcription of two complementary DNA strands, either in vivo, or in a single in vitro reaction mixture. See, e.g. U.S. Pat. No. 5,795,715. In one embodiment, dsRNA can be introduced into a plant or plant cell directly by standard transformation procedures. Alternatively, dsRNA can be expressed in a plant cell by transcribing two complementary RNAs.

Other methods for the inhibition of endogenous gene expression, such as triple helix formation (Moser et al., Science 238, 645 (1987), and Cooney et al., Science 241, 456 (1988)) and co-suppression (Napoli et al., The Plant Cell 2,279, 1990,) are known in the art. Partial and full-length cDNAs have been used for the c-osuppression of endogenous plant genes. See, e.g. U.S. Pat. Nos. 4,801,340, 5,034,323, 5,231,020, and 5,283,184; Van der Kroll et al., The Plant Cell 2, 291, (1990); Smith et al., Mol. Gen. Genetics 224, 477 (1990), and Napoli et al., The Plant Cell 2, 279 (1990).

For sense suppression, it is believed that introduction of a sense polynucleotide blocks transcription of the corresponding target gene. The sense polynucleotide will have at least 65% sequence identity with the target plant gene or RNA. Preferably, the percent identity is at least 80%, 90%, 95% or more. The introduced sense polynucleotide need not be full length relative to the target gene or transcript. Preferably, the sense polynucleotide will have at least 65% sequence identity with at least 100 consecutive nucleotides of one of the nucleic acids as depicted in SEQ ID NO: 1, 3, 5, 7, 26, or 44, or a homolog thereof. The regions of identity can comprise introns and/or exons and untranslated regions. The introduced sense polynucleotide may be present in the plant cell transiently, or may be stably integrated into a plant chromosome or extra-chromosomal replicon.

Further, embodiment of the invention is an expression vector comprising a nucleic acid molecule comprising a nucleic acid molecule selected from the group consisting of:
(a) a nucleic acid molecule encoding the polypeptide comprising the sequence of SEQ ID NO: 2, 4, 6, 8, 27, or 45, or a homolog thereof;
(b) a nucleic acid molecule comprising the sequence of SEQ ID NO: 1, 3, 5, 7, 26, or 44, or a homolog thereof,
(c) a nucleic acid molecule, which, as a result of the degeneracy of the genetic code, can be derived from a polypeptide sequence of SEQ ID NO: 2, 4, 6, 8, 27, or 45, or a homolog thereof, and confers increased herbicide tolerance or resistance, as compared to a corresponding, e.g. non-transformed, wild type plant cell, a plant or a part thereof;
(d) a nucleic acid molecule having 30% or more identity, preferably 40%, 50%, 60%, 70%, 75%, 80%, 85%, 90%, 95%, 96%, 97%, 98%, 99%, 99.5%, or more with the nucleic acid molecule sequence of a polynucleotide comprising the nucleic acid molecule of SEQ ID NO: 1, 3, 5, 7, 26, or 44, or a homolog thereof, and confers increased herbicide tolerance or resistance, as compared to a corresponding, e.g. non-transformed, wild type plant cell, a plant or a part thereof;
(e) a nucleic acid molecule encoding a polypeptide having 30% or more identity, preferably at least 40%, 50%, 60%, 70%, 75%, 80%, 85%, 90%, 95%, 96%, 97%, 98%, 99%, 99.5% or more, with the amino acid sequence of the polypeptide encoded by the nucleic acid molecule of (a), (b), (c) or (d) and having the activity represented by a nucleic acid molecule comprising a polynucleotide of SEQ ID NO: 1, 3, 5, 7, 26, or 44, or a homolog thereof, and confers increased herbicide tolerance or resistance as compared to a corresponding, e.g. non-transformed, wild type plant cell, a plant or a part thereof;
(f) nucleic acid molecule which hybridizes with a nucleic acid molecule of (a), (b), (c), (d) or (e) under stringent hybridization conditions and confers increased herbicide tolerance or resistance, as compared to a corresponding, e.g. non-transformed, wild type plant cell, a plant or a part thereof;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f) and having the activity represented by the nucleic acid molecule comprising a polynucleotide as depicted in SEQ ID NO: 1, 3, 5, 7, 26, or 44, or a homolog thereof;
(h) a nucleic acid molecule which is obtainable by screening a suitable nucleic acid library, especially a cDNA library and/or a genomic library, under stringent hybridization conditions with a probe comprising a complementary sequence of a nucleic acid molecule of (a) or (b) or with a fragment thereof, having 15 nt, preferably 20 nt, 30 nt, 50 nt, 100 nt, 200 nt, 500 nt, 750 nt or 1000 nt or more of a nucleic acid molecule complementary to a nucleic acid molecule sequence characterized in (a) to (e) and encoding a polypeptide having the activity represented by a protein comprising a polypeptide as depicted SEQ ID NO: 2, 4, 6, 8, 27, or 45, or a homolog thereof.

The invention further provides an isolated recombinant expression vector comprising the nucleic acid molecule of the invention, wherein expression of the vector or nucleic acid molecule, respectively in a host cell results in an increased herbicide tolerance or resistance, as compared to the corresponding, e.g. non-transformed, wild type of the host cell.

A plant expression cassette preferably contains regulatory sequences capable of driving gene expression in plant cells and operably linked so that each sequence can fulfill its function, for example, termination of transcription by polyadenylation signals. Preferred polyadenylation signals are those originating from Agrobacterium tumefaciens T-DNA such as the gene 3 known as octopine synthase of the Ti-plasmid pTiACH5 (Gielen et al., EMBO J. 3, 835 1(984)) or functional equivalents thereof but also all other terminators functionally active in plants are suitable. As plant gene expression is very often not limited on transcriptional levels, a plant expression cassette preferably contains other operably linked sequences like translational enhancers such as the overdrive-sequence containing the 5″-untranslated leader sequence from tobacco mosaic virus enhancing the protein per RNA ratio (Gallie et al., Nucl. Acids Research 15, 8693 (1987)).

Plant gene expression has to be operably linked to an appropriate promoter conferring gene expression in a timely, cell or tissue specific manner. Preferred are promoters driving constitutive expression (Benfey et al., EMBO J. 8, 2195 (1989)) like those derived from plant viruses like the 35S CaMV (Franck et al., Cell 21, 285 (1980)), the 19S CaMV (see also U.S. Pat. No. 5,352,605 and PCT Application No. WO 84/02913) or plant promoters like those from Rubisco small subunit described in U.S. Pat. No. 4,962,028. Other promoters, e.g. super-promoter (Ni et al., Plant Journal 7, 661 (1995)), Ubiquitin promoter (Callis et al., J. Biol. Chem., 265, 12486 (1990); U.S. Pat. No. 5,510,474; U.S. Pat. No. 6,020,190; Kawalleck et al., Plant. Molecular Biology, 21, 673 (1993)) or 34S promoter (GenBank Accession numbers M59930 and X16673) were similar useful for the present invention and are known to a person skilled in the art. Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters, and the like. Seed preferred promoters are preferentially expressed during seed development and/or germination. For example, seed preferred promoters can be embryo-preferred, endosperm preferred, and seed coat-preferred. See Thompson et al., BioEssays 10, 108 (1989). Examples of seed preferred promoters include, but are not limited to, cellulose synthase (celA), Cim1, gamma-zein, globulin-1, maize 19 kD zein (cZ19B1), and the like.

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2 and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

Additional advantageous regulatory sequences are, for example, included in the plant promoters such as CaMV/35S (Franck et al., Cell 21 285 (1980)), PRP1 (Ward et al., Plant. Mol. Biol. 22, 361 (1993)), SSU, OCS, lib4, usp, STLS1, B33, LEB4, nos, ubiquitin, napin or phaseolin promoter. Also advantageous in this connection are inducible promoters such as the promoters described in EP 388 186 (benzyl sulfonamide inducible), Gatz et al., Plant J. 2, 397 (1992) (tetracyclin inducible), EP-A-0 335 528 (abscisic acid inducible) or WO 93/21334 (ethanol or cyclohexenol inducible). Additional useful plant promoters are the cytoplasmic FBPase promoter or ST-LSI promoter of potato (Stockhaus et al., EMBO J. 8, 2445 (1989)), the phosphorybosyl phyrophoshate amido transferase promoter of Glycine max (gene bank accession No. U87999) or the noden specific promoter described in EP-A-0 249 676. Additional particularly advantageous promoters are seed specific promoters which can be used for monocotyledones or dicotyledones and are described in U.S. Pat. No. 5,608,152 (napin promoter from rapeseed), WO 98/45461 (phaseolin promoter from Arabidopsis), U.S. Pat. No. 5,504,200 (phaseolin promoter from Phaseolus vulgaris), WO 91/13980 (Bce4 promoter from Brassica) and Baeumlein et al., Plant J., 2 (2), 233 (1992) (LEB4 promoter from leguminosa). Said promoters are useful in dicotyledones. The following promoters are useful for example in monocotyledones Ipt-2- or Ipt-1-promoter from barley (WO 95/15389 and WO 95/23230) or hordein promoter from barley. Other useful promoters are described in WO 99/16890. It is possible in principle to use all natural promoters with their regulatory sequences like those mentioned above for the novel process. It is also possible and advantageous in addition to use synthetic promoters.

The gene construct may also comprise further genes which are to be inserted into the organisms and which are for example involved in herbicide tolerance or resistance increase. It is possible and advantageous to insert and express in host organisms regulatory genes such as genes for inducers, repressors or enzymes which intervene by their enzymatic activity in the regulation, or one or more or all genes of a biosynthetic pathway. These genes can be heterologous or homologous in origin. The inserted genes may have their own promoter or else be under the control of same promoter as the sequences of the nucleic acid of SEQ ID NO: 1, 3, 5, 7, 26, or 44, or their homologs.

The gene construct advantageously comprises, for expression of the other genes present, additionally 3′ and/or 5′ terminal regulatory sequences to enhance expression, which are selected for optimal expression depending on the selected host organism and gene or genes.

These regulatory sequences are intended to make specific expression of the genes and protein expression possible as mentioned above. This may mean, depending on the host organism, for example that the gene is expressed or over-expressed only after induction, or that it is immediately expressed and/or over-expressed.

The regulatory sequences or factors may moreover preferably have a beneficial effect on expression of the introduced genes, and thus increase it. It is possible in this way for the regulatory elements to be enhanced advantageously at the transcription level by using strong transcription signals such as promoters and/or enhancers. However, in addition, it is also possible to enhance translation by, for example, improving the stability of the mRNA.

Other preferred sequences for use in plant gene expression cassettes are targeting-sequences necessary to direct the gene product in its appropriate cell compartment (for review see Kermode, Crit. Rev. Plant Sci. 15 (4), 285 (1996) and references cited therein) such as the vacuole, the nucleus, all types of plastids like amyloplasts, chloroplasts, chromoplasts, the extracellular space, mitochondria, the endoplasmic reticulum, oil bodies, peroxisomes and other compartments of plant cells.

Plant gene expression can also be facilitated via an inducible promoter (for review see Gatz, Annu. Rev. Plant Physiol. Plant Mol. Biol. 48, 89 (1997)). Chemically inducible promoters are especially suitable if gene expression is wanted to occur in a time specific manner.

Table xx lists several examples of promoters that may be used to regulate transcription of the nucleic acid coding sequences of the present invention.

TABLE xx
Examples of tissue-specific and inducible promoters in plants

| Expression | Reference |
|---|---|
| Cor78 - Cold, drought, salt, | Ishitani, et al., Plant Cell 9, 1935 (1997), |
| ABA, wounding-inducible | Yamaguchi-Shinozaki and Shinozaki, Plant Cell 6, 251 |
| | (1994) |
| Rci2A - Cold, dehydration- | Capel et al., Plant Physiol 115, 569 (1997) |
| inducible | |
| Rd22 - Drought, salt | Yamaguchi-Shinozaki and Shinozaki, Mol. Gen. Genet. |
| | 238, 17 (1993) |
| Cor15A - Cold, dehydration, | Baker et al., Plant Mol. Biol. 24, 701 (1994) |
| ABA | |
| GH3 - Auxin inducible | Liu et al., Plant Cell 6, 645 (1994) |
| ARSK1-Root, salt inducible | Hwang and Goodman, Plant J. 8, 37 (1995) |
| PtxA - Root, salt inducible | GenBank accession X67427 |
| SbHRGP3 - Root specific | Ahn et al., Plant Cell 8, 1477 (1998). |
| KST1 - Guard cell specific | Plesch et al., Plant Journal. 28(4), 455-(2001) |
| KAT1 - Guard cell specific | Plesch et al., Gene 249, 83 (2000), |
| | Nakamura et al., Plant Physiol. 109, 371 (1995) |
| salicylic acid inducible | PCT Application No. WO 95/19443 |
| tetracycline inducible | Gatz et al., Plant J. 2, 397 (1992) |
| Ethanol inducible | PCT Application No. WO 93/21334 |
| Pathogen inducible PRP1 | Ward et al., Plant. Mol. Biol. 22, 361-(1993) |
| Heat inducible hsp80 | U.S. Pat. No. 5,187,267 |
| Cold inducible alpha- | PCT Application No. WO 96/12814 |
| amylase | |
| Wound-inducible pinII | European Patent No. 375 091 |
| RD29A - salt-inducible | Yamaguchi-Shinozalei et al. Mol. Gen. Genet. 236, 331 |
| | (1993) |
| Plastid-specific viral RNA- | PCT Application No. WO 95/16783, PCT Application |
| polymerase | WO 97/06250 |

Additional flexibility in controlling heterologous gene expression in plants may be obtained by using DNA binding domains and response elements from heterologous sources (i.e., DNA binding domains from non-plant sources). An example of such a heterologous DNA binding domain is the LexA DNA binding domain (Brent and Ptashne, Cell 43, 729 (1985)).

In one embodiment, the language “substantially free of cellular material” includes preparations of a protein having less than about 30% (by dry weight) of contaminating material (also referred to herein as a “contaminating polypeptide”), more preferably less than about 20% of contaminating material, still more preferably less than about 10% of contaminating material, and most preferably less than about 5% contaminating material.

The nucleic acid molecules, polypeptides, polypeptide homologs, fusion polypeptides, primers, vectors, and host cells described herein can be used in one or more of the following methods: identification of S. cerevisiae, E. coli or Brassica napus, Glycine max, Zea mays or Oryza sativa and related organisms; mapping of genomes of organisms related to S. cerevisiae, E. coli; identification and localization of S. cerevisiae, E. coli or Brassica napus, Glycine max, Zea mays or Oryza sativa sequences of interest; evolutionary studies; determination of polypeptide regions required for function; modulation of a polypeptide activity; modulation of the metabolism of one or more cell functions; modulation of the transmembrane transport of one or more compounds; modulation of herbicide tolerance or resistance, and modulation of expression of polypeptide nucleic acids.

The nucleic acid molecules of the invention are also useful for evolutionary and polypeptide structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the polypeptide that are essential for the functioning of the enzyme. This type of determination is of value for polypeptide engineering studies and may give an indication of what the polypeptide can tolerate in terms of mutagenesis without losing function.

There are a number of mechanisms by which the alteration of the polypeptide of the invention may directly affect herbicide tolerance or resistance.

The effect of the genetic modification in plants regarding herbicide tolerance or resistance can be assessed by treating the modified plant with respective herbicides as, e.g., described in EXAMPLE 4, and then analyzing the growth characteristics and/or metabolism of the plant in comparison to non-modified plants. Such analysis techniques are well known to one skilled in the art, and include evaluation of the plant phenotype, dry weight, fresh weight, polypeptide synthesis, carbohydrate synthesis, lipid synthesis, evapotranspiration rates, general plant and/or crop yield, flowering, reproduction, seed setting, root growth, respiration rates, photosynthesis rates, etc. (Applications of HPLC in Biochemistry in: Laboratory Techniques in Biochemistry and Molecular Biology, Vol. 17; Rehm et al., 1993 Biotechnology, Vol. 3, Chapter III: Product recovery and purification, page 469-714, VCH: Weinheim; Better P. A. et al., 1988, Bioseparations: downstream processing for biotechnology, John Wiley and Sons; Kennedy J. F., and Cabral J. M. S., 1992, Recovery processes for biological materials, John Wiley and Sons; Shaeiwitz J. A. and Henry J. D., 1988, Biochemical separations, in Ulmann's Encyclopedia of Industrial Chemistry, Vol. B3, Chapter 11, page 1-27, VCH: Weinheim; and Dechow F. J., 1989, Separation and purification techniques in biotechnology, Noyes Publications).

For example, plant expression vectors comprising the nucleic acids disclosed herein, or fragments thereof, can be constructed and transformed into an appropriate plant cell such as rape, maize, cotton, rice, wheat, sugar cane, sugar beet, soy bean, Arabidopsis thaliana, potato, Medicago truncatula, etc., using standard protocols. The resulting transgenic cells and/or plants derived therefrom can then be assayed for generation or alteration of their herbicide tolerance or resistance.

The present invention also provides antibodies that specifically bind to the polypeptide according to the invention, or a portion thereof, as encoded by a nucleic acid described herein. Antibodies can be made by many well-known methods (see, e.g. Harlow and Lane, “Antibodies; A Laboratory Manual”, Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. See, for example, Kelly et al., Bio/Technology 10, 163 (1992); Bebbington et al., Bio/Technology 10, 169 (1992).

Gene expression in plants is regulated by the interaction of protein transcription factors with specific nucleotide sequences within the regulatory region of a gene. One example of transcription factors are polypeptides that contain zinc finger (ZF) motifs. Each ZF module is approximately 30 amino acids long folded around a zinc ion. The DNA recognition domain of a ZF protein is a α-helical structure that inserts into the major grove of the DNA double helix. The module contains three amino acids that bind to the DNA with each amino acid contacting a single base pair in the target DNA sequence. ZF motifs are arranged in a modular repeating fashion to form a set of fingers that recognize a contiguous DNA sequence. For example, a three-fingered ZF motif will recognize 9 bp of DNA. Hundreds of proteins have been shown to contain ZF motifs with between 2 and 37 ZF modules in each protein (Isalan M. et al., Biochemistry 37 (35), 12026 (1998); Moore M. et al., Proc. Natl. Acad. Sci. USA 98 (4), 1432 (2001) and Moore M. et al., Proc. Natl. Acad. Sci. USA 98 (4), 1437 (2001); U.S. Pat. No. 6,007,988 and U.S. Pat. No. 6,013,453).

The regulatory region of a plant gene contains many short DNA sequences (cis-acting elements) that serve as recognition domains for transcription factors, including ZF proteins. Similar recognition domains in different genes allow the coordinate expression of several genes encoding enzymes in a metabolic pathway by common transcription factors. Variation in the recognition domains among members of a gene family facilitates differences in gene expression within the same gene family, for example, among tissues and stages of development and in response to environmental conditions.

Typical ZF proteins contain not only a DNA recognition domain but also a functional domain that enables the ZF protein to activate or repress transcription of a specific gene. Experimentally, an activation domain has been used to activate transcription of the target gene (U.S. Pat. No. 5,789,538 and patent application WO 95/19431), but it is also possible to link a transcription repressor domain to the ZF and thereby inhibit transcription (patent applications WO 00/47754 and WO 01/002019). It has been reported that an enzymatic function such as nucleic acid cleavage can be linked to the ZF (patent application WO 00/20622).

The invention provides a method that allows one skilled in the art to isolate the regulatory region of one or more polypeptide according to the invention-encoding genes from the genome of a plant cell and to design zinc finger transcription factors linked to a functional domain that will interact with the regulatory region of the gene. The interaction of the zinc finger protein with the plant gene can be designed in such a manner as to alter expression of the gene and preferably thereby to confer increasing herbicide tolerance or resistance.

In particular, the invention provides a method of producing a transgenic plant with a coding nucleic acid, wherein expression of the nucleic acid(s) in the plant results in increasing herbicide tolerance or resistance, as compared to a wild type plant comprising: (a) transforming a plant cell with an expression vector comprising a encoding nucleic acid, and (b) generating from the plant cell a transgenic plant with enhanced increased herbicide tolerance or resistance as compared to a wild type plant. For such plant transformation, binary vectors such as pBinAR can be used (Höfgen and Willmitzer, Plant Science 66, 221 (1990)). Moreover suitable binary vectors are for example pBIN19, pBI101, pGPTV or pPZP (Hajukiewicz P. et al., Plant Mol. Biol., 25, 989 (1994)).

Alternate methods of transfection include the direct transfer of DNA into developing flowers via electroporation or Agrobacterium mediated gene transfer. Agrobacterium mediated plant transformation can be performed using for example the GV3101 (pMP90) (Koncz and Schell, Mol. Gen. Genet. 204, 383 (1986)) or LBA4404 (Ooms et al., Plasmid, 7, 15 (1982); Hoekema et al., Nature, 303, 179 (1983)) Agrobacterium tumefaciens strain. Transformation can be performed by standard transformation and regeneration techniques (Deblaere et al., Nucl. Acids. Res. 13, 4777 (1994); Gelvin and Schilperoort, Plant Molecular Biology Manual, 2nd Ed.—Dordrecht: Kluwer Academic Publ., 1995.—in Sect., Ringbuc Zentrale Signatur: BT11-P ISBN 0-7923-2731-4; Glick B. R. and Thompson J. E., Methods in Plant Molecular Biology and Biotechnology, Boca Raton: CRC Press, 1993.—360 S., ISBN 0-8493-5164-2). For example, rapeseed can be transformed via cotyledon or hypocotyl transformation (Moloney et al., Plant Cell Reports 8, 238 (1989); De Block et al., Plant Physiol. 91, 694 (1989)). Use of antibiotics for Agrobacterium and plant selection depends on the binary vector and the Agrobacterium strain used for transformation. Rapeseed selection is normally performed using kanamycin as selectable plant marker. Agrobacterium mediated gene transfer to flax can be performed using, for example, a technique described by Mlynarova et al., Plant Cell Report 13, 282 (1994)). Additionally, transformation of soybean can be performed using for example a technique described in European Patent No. 424 047, U.S. Pat. No. 5,322,783, European Patent No. 397 687, U.S. Pat. No. 5,376,543 or U.S. Pat. No. 5,169,770. Transformation of maize can be achieved by particle bombardment, polyethylene glycol mediated DNA uptake or via the silicon carbide fiber technique (see, for example, Freeling and Walbot “The maize handbook” Springer Verlag: New York (1993) ISBN 3-540-97826-7). A specific example of maize transformation is found in U.S. Pat. No. 5,990,387 and a specific example of wheat transformation can be found in PCT Application No. WO 93/07256.

In one embodiment, the present invention relates to a method for the identification of a gene product conferring in increasing herbicide tolerance or resistance, as compared to a corresponding, e.g. non-transformed, wild type cell in a cell of an organism for example plant, comprising the following steps:
(a) contacting, e.g. hybridizing, some or all nucleic acid molecules of a sample, e.g. cells, tissues, plants or microorganisms or a nucleic acid library, which can contain a candidate gene encoding a gene product conferring increased herbicide tolerance or resistance with a nucleic acid molecule as shown in SEQ ID NO: 1, 3, 5, 7, 26, or 44, or a functional homologue thereof;
(b) identifying the nucleic acid molecules, which hybridize under relaxed stringent conditions with said nucleic acid molecule, in particular to the nucleic acid molecule sequence shown in SEQ ID NO: 1, 3, 5, 7, 26, or 44, or a homolog thereof, and, optionally, isolating the full length cDNA clone or complete genomic clone;
(c) identifying the candidate nucleic acid molecules or a fragment thereof in host cells, preferably in a plant cell;
(d) increasing the expressing of the identified nucleic acid molecules in the host cells for which increased herbicide tolerance or resistance are desired;
(e) assaying the level of increased herbicide tolerance or resistance of the host cells; and
(f) identifying the nucleic acid molecule and its gene product which confers increased herbicide tolerance or resistance, in the host cell compared to the wild type.

Relaxed hybridization conditions are: After standard hybridization procedures washing steps can be performed at low to medium stringency conditions usually with washing conditions of 40°-55° C. and salt conditions between 2×SSC and 0,2×SSC with 0,1% SDS in comparison to stringent washing conditions as e.g. 60° to 68° C. with 0,1% SDS. Further examples can be found in the references listed above for the stringend hybridization conditions. Usually washing steps are repeated with increasing stringency and length until a useful signal to noise ratio is detected and depend on many factors as the target, e.g. its purity, GC-content, size etc, the probe, e.g. its length, is it a RNA or a DNA probe, salt conditions, washing or hybridization temperature, washing or hybridization time etc.

In another embodiment, the present invention relates to a method for the identification of a gene product the expression of which confers increased herbicide tolerance or resistance, in a cell, comprising the following steps:
(a) identifying a nucleic acid molecule in an organism, which is at least 20%, preferably 25%, more preferably 30%, even more preferred are 35%. 40% or 50%, even more preferred are 60%, 70% or 80%, most preferred are 90% or 95% or more homolog to the nucleic acid molecule encoding a protein comprising the polypeptide molecule as shown in SEQ ID NO: 2, 4, 6, 8, 27, or 45, or a homolog therefor being encoded by a nucleic acid molecule comprising a polynucleotide as shown in SEQ ID NO: 1, 3, 5, 7, 26, or 44, or a homologue thereof as described herein, for example via homology search in a data bank;
(b) enhancing the expression of the identified nucleic acid molecules in the host cells;
(c) assaying the level of enhancement of in increasing herbicide tolerance or resistance, in the host cells; and
(d) identifying the host cell, in which the enhanced expression confers in increasing herbicide tolerance or resistance, in the host cell compared to a wild type.

Further, the nucleic acid molecule disclosed herein, in particular the nucleic acid molecule shown in SEQ ID NO: 1, 3, 5, 7, 26, or 44, may be sufficiently homologous to the sequences of related species such that these nucleic acid molecules may serve as markers for the construction of a genomic map in related organism or for association mapping. Furthermore natural variation in the genomic regions corresponding to nucleic acids disclosed herein, in particular the nucleic acid molecule shown in SEQ ID NO: 1, 3, 5, 7, 26, or 44, or homologous thereof may lead to variation in the activity of the proteins disclosed herein, in particular the proteins comprising polypeptides as shown in SEQ ID NO: 2, 4, 6, 8, 27, or 45, and their homolgous and in consequence in a natural variation of an increased herbicide tolerance or resistance.

In consequence natural variation eventually also exists in form of more active allelic variants leading already to a relative increase in herbicide tolerance or resistance. Different variants of the nucleic acids molecule disclosed herein, in particular the nucleic acid comprising the nucleic acid molecule as shown SEQ ID NO: 1, 3, 5, 7, 26, or 44, or a homolog thereof, which corresponds to different levels of increased herbicide tolerance or resistance can be identified and used for marker assisted breeding for an increased herbicide tolerance or resistance,

Accordingly, the present invention relates to a method for breeding plants with an increased herbicide tolerance or resistance, comprising
(a) selecting a first plant variety with an increased herbicide tolerance or resistance, based on increased expression of a nucleic acid of the invention as disclosed herein, in particular of a nucleic acid molecule comprising a nucleic acid molecule as shown in SEQ ID NO: 1, 3, 5, 7, 26, or 44, or a homolog thereof, or a polypeptide comprising a polypeptide as shown in SEQ ID NO: 2, 4, 6, 8, 27, or 45, or a homolog thereof, or a homologue thereof as described herein;
(b) associating the level of increased herbicide tolerance or resistance with the expression level or the genomic structure of a gene encoding said polypeptide or said nucleic acid molecule;
(c) crossing the first plant variety with a second plant variety, which significantly differs in its level of increased herbicide tolerance or resistance; and
(d) identifying, which of the offspring varieties has got increased levels of herbicide tolerance or resistance,

In another embodiment, the present invention relates to a kit comprising the nucleic acid molecule, the vector, the host cell, the polypeptide, or the antisense, RNAi, snRNA, dsRNA, siRNA, miRNA, ta-siRNA, cosuppression molecule, or ribozyme molecule, or the viral nucleic acid molecule, the antibody, plant cell, the plant or plant tissue, the harvestable part, the propagation material and/or the compound and/or agonist identified according to the method of the invention.

The compounds of the kit of the present invention may be packaged in containers such as vials, optionally with/in buffers and/or solution. If appropriate, one or more of said components might be packaged in one and the same container. Additionally or alternatively, one or more of said components might be adsorbed to a solid support as, e.g. a nitrocellulose filter, a glas plate, a chip, or a nylon membrane or to the well of a micro titerplate. The kit can be used for any of the herein described methods and embodiments, e.g. for the production of the host cells, transgenic plants, pharmaceutical compositions, detection of homologous sequences, identification of antagonists or agonists, as food or feed or as a supplement thereof or as supplement for the treating of plants, etc. Further, the kit can comprise instructions for the use of the kit for any of said embodiments. In one embodiment said kit comprises further a nucleic acid molecule encoding one or more of the aforementioned protein, and/or an antibody, a vector, a host cell, an antisense nucleic acid, a plant cell or plant tissue or a plant. In another embodiment said kit comprises PCR primers to detect and discriminate the nucleic acid molecule to be reduced in the process of the invention, e.g. of the nucleic acid molecule of the invention.

In a further embodiment, the present invention relates to a method for the production of an agricultural composition providing the nucleic acid molecule for the use according to the process of the invention, the nucleic acid molecule of the invention, the vector of the invention, the antisense, RNAi, snRNA, dsRNA, siRNA, miRNA, ta-siRNA, cosuppression molecule, ribozyme, or antibody of the invention, the viral nucleic acid molecule of the invention, or the polypeptide of the invention or comprising the steps of the method according to the invention for the identification of said compound or agonist; and formulating the nucleic acid molecule, the vector or the polypeptide of the invention or the agonist, or compound identified according to the methods or processes of the present invention or with use of the subject matters of the present invention in a form applicable as plant agricultural composition.

In another embodiment, the present invention relates to a method for the production of the plant culture composition comprising the steps of the method of the present invention; and formulating the compound identified in a form acceptable as agricultural composition.

Under “acceptable as agricultural composition” is understood, that such a composition is in agreement with the laws regulating the content of fungicides, plant nutrients, herbicides, etc. Preferably such a composition is without any harm for the protected plants and the animals (humans included) fed therewith. Said polypeptide or nucleic acid molecule or the genomic structure of the genes encoding said polypeptide or nucleic acid molecule of the invention.

Throughout this application, various publications are referenced. The disclosures of all of these publications and those references cited within those publications in their entireties are hereby incorporated by reference into this application in order to more fully describe the state of the art to which this invention pertains.

It should also be understood that the foregoing relates to preferred embodiments of the present invention and that numerous changes and variations may be made therein without departing from the scope of the invention. The invention is further illustrated by the following examples, which are not to be construed in any way as limiting. On the contrary, it is to be clearly understood that various other embodiments, modifications and equivalents thereof, which, after reading the description herein, may suggest themselves to those skilled in the art without departing from the spirit of the present invention and/or the scope of the claims.

In one embodiment, the increased herbicide tolerance or resistance results in an increase of the production of a specific ingredient including, without limitation, an enhanced and/or improved sugar content or sugar composition, an enhanced or improved starch content and/or starch composition, an enhanced and/or improved oil content and/or oil composition (such as enhanced seed oil content), an enhanced or improved protein content and/or protein composition (such as enhanced seed protein content), an enhanced and/or improved vitamin content and/or vitamin composition, or the like.

### EXAMPLES

### Example 1

### Identification of Genes Encoding Enzymes with Activity Towards Compound Metabolism

Isolation of RNA and cDNA Synthesis

Leaf tissue of Alopecurus myosurodes was harvested, frozen and grounded in liquid nitrogen and total RNA was extracted using an Ambion RNAqueous-Midi kit (AM1911, Ambion) with the Plant RNA Isolation Aid (AM9690, Ambion) as per manufacturer's recommendation. The last elution was done with 10 ul of elution solution. To validate the quality of the extracted RNA 1 uL of the final product was run on a Bioanalyzer 2100 using the RNA 6000 Nano kit with the Plant RNA Nano method. The final solution, containing purified RNA, was stored at −80° C. until library preparation.

RNA sequencing libraries were produced using TruSeq RNA Sample preparation kits V2 (RS-122-2001) from Illumina according to the instructions of the manufacturer. Briefly, 1 μg of total RNA was first purified twice on a poly-dT column. During the second elution step, RNA was fragmented and primed for cDNA synthesis. The material was reverse transcribed, RNA was removed and the second strand was produced. After rendering the ends of the fragment blunt, 3′ ends were adenylated and Illumina sequencing-specific bar-coded adaptors were ligated at both ends of the fragments. The DNA fragments bearing adaptors at both ends were enriched by 15 cycle PCR amplification. Libraries are pooled prior to sequencing.

### Sequencing

The pooled libraries were first put on a flowcell using a TruSeq PE Cluster kit V3 (PE-401-3001) on the cBot and clusters are amplified on the device. Afterwards, the flowcell is transferred onto the Illumina Hiseq machine and the material on the flowcell is then sequenced using Illumina TruSeq SBS Kit V3 (FC-401-3001) as per manufacturer's recommendation.

### EST Assembly and Calculation of Expression Level

The data produced by the Illumina Hiseq sequencer was first trimmed at both ends using a quality threshold of 15 using the FASTQC Quality Trimmer (http://www.bioinformatics.babraham.ac.uk/projects/fastqc/). These sequences were further analyzed to remove any Illumina adaptor sequences using CutAdapt (http://code.google.com/p/cutadapt/). Sequence reads were assembled using CLC bio algorithm (version 4.01). Short read alignments were performed using the software Tophat (http://tophat.cbcb.umd.edu/) and expression values were calculated and compared using Cufflinks (http://cufflinks.cbcb.umd.edu/).

### Example 2

### Detection of Herbicide Degradation by Biochemical Assay

### Yeast Expression System:

The cDNA of CYP450 monooxygenase genes were synthesized with an optimized codon usage for yeast, cloned via unique BamHI-SalI restriction sites in the low copy pESC-ura expression vector (Agilent Technologies). Constructs were transformed into S. cerevisiae wild type strain BJ5459 (MATa ura3-5 trp lys2-801 leu2Δ1 his 3Δ200 pep4Δ::HIS3 prb1Δ1.6R can1 GAL cir⁺; ATCC 208284) using a Yeast Maker Transformation System from Clontech and verified by colony PCR. Positive clones were selected on minimal synthetic-defined media (SD) supplemented with appropriate dropout solution. The strain had no obvious phenotypes. Cells were induced in SG-Ura medium (same composition as SD but with galactose instead of glucose) for 24 h (Pompon et al., Methods in Enzymology 272:51-64 (1996); Urban et al., Eur. J. Biochem. 222:853-850 (1994)). Optimal heterologous protein expression was assayed using Western Blot analysis.

### Analysis of Xenobiotic Metabolism:

96 deep well growth plates (STARLAB GmbH) charged with 700 μL SDA medium are inoculated with the respective yeast strains from cryostock and incubated at 30° C., 400 rpm. After 48 h, an aliquot is transferred into a new plate with fresh SDA medium 400 rpm. After 4 h the cultures are spun down, the supernatant discarded and the pellets resuspended in 700 μL pre-warmed SGA media to induce protein expression at 30° C. and 400 rpm. After an incubation time of 24 h, 7 μL herbicide solution (500 μM DMSO stock solution) or solvent control is added to the yeast culture incubated for additional 24 h. The herbicide conversion is stopped by adding 700 μL acetonitrile followed by ultrasonification. The homogenate is prepared for UPLC-MS/MS analysis. The degradation rate was calculated by the determination of the recovery of the herbicide in reference to the control.

### Example 3

### Engineering Herbicide Tolerant Plants Having Additional Cytochrome P450 Genes

Herbicide tolerant soybean (Glycine max) or corn (Zea mays) plants are generated as described by Olhoft et al. (US patent 2009/0049567). For transformation of soybean or Arabidopsis thaliana, CYP450 monooxygenase genes of the present invention are cloned with standard cloning techniques as described in Sambrook et al. (Molecular cloning (2001) Cold Spring Harbor Laboratory Press) in a binary vector containing resistance marker gene cassette (AHAS) and CYP450 monooxygenase sequence (marked as GOI) in between ubiquitin promoter (PcUbi) and nopaline synthase terminator (NOS) sequence. For corn transformation, CYP450 monooxygenase sequences are cloned with standard cloning techniques as described in Sambrook et al. (Molecular cloning (2001) Cold Spring Harbor Laboratory Press) in a binary vector containing resistance marker gene cassette (AHAS) and CYP450 monooxygenase sequence (marked as GOI) in between corn ubiquitin promoter (ZmUbi) and nopaline synthase terminator (NOS) sequence. Binary plasmids are introduced to Agrobacterium tumefaciens for plant transformation. Plasmid constructs are introduced into soybean's axillary meristem cells at the primary node of seedling explants via Agrobacterium-mediated transformation. After inoculation and co-cultivation with Agrobacteria, the explants are transferred to shoot introduction media without selection for one week. The explants were subsequently transferred to a shoot induction medium with 1-3 μM imazapyr (Arsenal) for 3 weeks to select for transformed cells. Explants with healthy callus/shoot pads at the primary node are then transferred to shoot elongation medium containing 1-3 μM imazapyr until a shoot elongated or the explant died. Transgenic plantlets are rooted, subjected to TaqMan analysis for the presence of the transgene, transferred to soil and grown to maturity in greenhouse. Transformation of corn plants are done by a method described by McElver and Singh (WO 2008/124495). Plant transformation vector constructs containing CYP450 monooxygenase sequences are introduced into maize immature embryos via Agrobacterium-mediated transformation.

Transformed cells were selected in selection media supplemented with 0.5-1.5 μM ima-zethapyr for 3-4 weeks. Transgenic plantlets were regenerated on plant regeneration media and rooted afterwards. Transgenic plantlets are subjected to TaqMan analysis for the presence of the transgene before being transplanted to potting mixture and grown to maturity in greenhouse. Arabidopsis thaliana are transformed with CYP450 monooxygenase sequences by floral dip method as described by McElver and Singh (WO 2008/124495). Transgenic Arabidopsis plants were subjected to TaqMan analysis for analysis of the number of integration loci. Transformation of Oryza sativa (rice) are done by protoplast transformation as described by Peng et al. (U.S. Pat. No. 6,653,529)

### Example 4

### Demonstration of Herbicide Tolerance

T0 or T1 transgenic plant of soybean, corn, and rice containing CYP450 monooxygenase sequences are tested for improved tolerance to herbicides in greenhouse studies and miniplot studies with the following herbicides: saflufenacil, benzoxazinone-derivative herbicide, flumioxazin, butafenacil, acifluorfen, lactofen, bifenox, diuron, sulfentrazone, tepraloxydim, coumarone-derivative herbicides, azine-derivative herbicides.

For the pre-emergence treatment, the herbicides are applied directly after sowing by means of finely distributing nozzles. The containers are irrigated gently to promote germination and growth and subsequently covered with transparent plastic hoods until the plants have rooted. This cover causes uniform germination of the test plants, unless this has been impaired by the herbicides. For post emergence treatment, the test plants are first grown to a height of 3 to 15 cm, depending on the plant habit, and only then treated with the herbicides. For this purpose, the test plants are either sown directly and grown in the same containers, or they are first grown separately and transplanted into the test containers a few days prior to treatment.

For testing of T0 plants, cuttings can be used. In the case of soybean plants, an optimal shoot for cutting is about 7.5 to 10 cm tall, with at least two nodes present. Each cutting is taken from the original transformant (mother plant) and dipped into rooting hormone powder (indole-3-butyric acid, IBA). The cutting is then placed in oasis wedges inside a bio-dome. Wild type cuttings are also taken simultaneously to serve as controls. The cuttings are kept in the bio-dome for 5-7 days and then transplanted to pots and then acclimated in the growth chamber for two more days. Subsequently, the cuttings are transferred to the greenhouse, acclimated for approximately 4 days, and then subjected to spray tests as indicated. Depending on the species, the plants are kept at 10-25° C. or 20-35° C. The test period extends over 3 weeks. During this time, the plants are tended and their response to the individual treatments is evaluated. Herbicide injury evaluations are taken at 2 and 3 weeks after treatment. Plant injury is rated on a scale of 0% to 100%, 0% being no injury and 100% being complete death.

Transgenic Arabidopsis thaliana plants were assayed for improved tolerance to saflufenacil, benzoxazine-derivative herbicide, flumioxazin, butafenacil, acifluorfen, lactofen, bifenox, diuron, sulfentrazon, tepraloxydim, coumarone-derivative herbicides, azine-derivative herbicides in 48-well plates. Therefore, T2 seeds are surface sterilized by stirring for 5 min in ethanol+water (70+30 by volume), rinsing one time with ethanol+water (70+30 by volume) and two times with sterile, deionized water. The seeds are resuspended in 0.1% agar dissolved in water (w/v) Four to five seeds per well are plated on solid nutrient medium consisting of half-strength murashige skoog nutrient solution, pH 5.8 (Murashige and Skoog (1962) Physiologia Plantarum 15: 473-497). Compounds are dissolved in dimethylsulfoxide (DMSO) and added to the medium prior solidification (final DMSO concentration 0.1%). Multi well plates are incubated in a growth chamber at 22° C., 75% relative humidity and 110 μmol Phot*m⁻²*s⁻¹ with 14:10 h light:dark photoperiod. Growth inhibition is evaluated seven to ten days after seeding in comparison to wild type plants. Additionally, transgenic T1 Arabidopsis plants were tested for improved tolerance to herbicides in greenhouse studies with the following herbicides: saflufenacil, benzoxazinone-derivative herbicide, flumioxazin, butafenacil, acifluorfen, lactofen, bifenox, diuron, sulfentrazone, tepraloxydim, coumarone-derivative herbicides, azine-derivative herbicides.

### Example 5

### Sequence Analysis

Leaf tissue was collected from clonal plants separated for transplanting and analyzed as individuals. Genomic DNA was extracted using a Wizard® 96 Magnetic DNA Plant System kit (Promega, U.S. Pat. Nos. 6,027,945 & 6,368,800) as directed by the manufacturer. Isolated DNA was PCR amplified using the appropriate forward and reverse primer. PCR amplification was performed using Hotstar Taq DNA Polymerase (Qiagen) using touchdown thermocycling program as follows: 96° C. for 15 min, followed by 35 cycles (96° C., 30 sec; 58° C.-0.2° C. per cycle, 30 sec; 72° C., 3 min and 30 sec), 10 min at 72° C. PCR products were verified for concentration and fragment size via agarose gel electrophoresis. Dephosphorylated PCR products were analyzed by direct sequence using the PCR primers (DNA Landmarks, or Entelechon). Chromatogram trace files (.scf) were analyzed for mutation relative to the wild-type gene using Vector NTI Advance 10™ (Invitrogen). Based on sequence information, mutations were identified in several individuals. Sequence analysis was performed on the representative chromatograms and corresponding AlignX alignment with default settings and edited to call secondary peaks.

1. A method for producing a transgenic plant with increased herbicide tolerance or resistance as compared to a corresponding non-transformed wild type plant, comprising transforming a plant cell or a plant cell nucleus or a plant tissue with a nucleic acid molecule comprising a nucleic acid molecule selected from the group consisting of:
(a) an isolated polynucleotide encoding the polypeptide comprising the sequence of SEQ ID NO: 2, 4, 6, 8, 27, or 45, or a homolog thereof;
(b) an isolated polynucleotide comprising the sequence of SEQ ID NO: 1, 3, 5, 7, 26, or 44, or a homolog thereof;
(c) an isolated polynucleotide, which, as a result of the degeneracy of the genetic code, can be derived from a polypeptide comprising the sequence of SEQ ID NO: 2, 4, 6, 8, 27, or 45, or a homolog thereof and confers an increased herbicide tolerance or resistance as compared to a corresponding, e.g. non-transformed, wild type plant cell, a transgenic plant or a part thereof;
(d) an isolated polynucleotide having 30% or more identity with the sequence of a polynucleotide comprising the sequence of SEQ ID NO: 1, 3, 5, 7, 26, or 44, or a homolog thereof and conferring an increased herbicide tolerance or resistance as compared to a corresponding, e.g. non-transformed, wild type plant cell, a transgenic plant or a part thereof;
(e) an isolated polynucleotide encoding a polypeptide having 30% or more identity with the amino acid sequence of the polypeptide encoded by the isolated polynucleotide of (a) to (c) and conferring an increased herbicide tolerance or resistance as compared to a corresponding, e.g. non-transformed, wild type plant cell, a transgenic plant or a part thereof;
(f) an isolated polynucleotide which hybridizes with an isolated polynucleotide of (a) to (c) under stringent hybridization conditions and confers an increased herbicide tolerance or resistance as compared to a corresponding, e.g. non-transformed, wild type plant cell, a transgenic plant or a part thereof;
(g) an isolated polynucleotide encoding a polypeptide which can be isolated with the aid of monoclonal or polyclonal antibodies made against a polypeptide encoded by one of the isolated polynucleotides of (a) to (e) and which has the activity represented by the polynucleotide comprising the sequence of SEQ ID NO: 1, 3, 5, 7, 26, or 44, or a homolog thereof,
and regenerating a transgenic plant from that transformed plant cell nucleus, plant cell or plant tissue with increased herbicide tolerance or resistance.

2. An isolated nucleic acid molecule comprising a nucleic acid molecule selected from the group consisting of:
(a) a nucleic acid molecule encoding the polypeptide comprising the sequence of SEQ ID NO: 2, 4, 6, 8, 27, or 45, or a homolog thereof;
(b) a nucleic acid molecule comprising the sequence of SEQ ID NO: 1, 3, 5, 7, 26, or 44, or a homolog thereof,
(c) a nucleic acid molecule, which, as a result of the degeneracy of the genetic code, can be derived from a polypeptide sequence of SEQ ID NO: 2, 4, 6, 8, 27, or 45, or a homolog thereof, and confers increased herbicide tolerance or resistance, as compared to a corresponding, e.g. non-transformed, wild type plant cell, a plant or a part thereof;
(d) a nucleic acid molecule having 30% or more identity with the nucleic acid molecule sequence of a polynucleotide comprising the nucleic acid molecule of SEQ ID NO: 1, 3, 5, 7, 26, or 44, or a homolog thereof, and confers increased herbicide tolerance or resistance, as compared to a corresponding, e.g. non-transformed, wild type plant cell, a plant or a part thereof;
(e) a nucleic acid molecule encoding a polypeptide having 30% or more identity with the amino acid sequence of the polypeptide encoded by the nucleic acid molecule of (a), (b), (c) or (d) and having the activity represented by a nucleic acid molecule comprising a polynucleotide of SEQ ID NO: 1, 3, 5, 7, 26, or 44, or a homolog thereof, and confers increased herbicide tolerance or resistance as compared to a corresponding non-transformed, wild type plant cell, plant or part thereof;
(f) nucleic acid molecule which hybridizes with a nucleic acid molecule of (a), (b), (c), (d) or (e) under stringent hybridization conditions and confers increased herbicide tolerance or resistance, as compared to a corresponding non-transformed, wild type plant cell, plant or part thereof;
(g) a nucleic acid molecule encoding a polypeptide which can be isolated with the aid of monoclonal or polyclonal antibodies made against a polypeptide encoded by one of the nucleic acid molecules of (a), (b), (c), (d), (e) or (f) and having the activity represented by the nucleic acid molecule comprising a polynucleotide as depicted in SEQ ID NO: 1, 3, 5, 7, 26, or 44, or a homolog thereof;
(h) a nucleic acid molecule which is obtainable by screening a suitable nucleic acid library, especially a cDNA library and/or a genomic library, under stringent hybridization conditions with a probe comprising a complementary sequence of a nucleic acid molecule of (a) or (b) or with a fragment thereof, having 15 nt, preferably 20 nt, 30 nt, 50 nt, 100 nt, 200 nt, 500 nt, 750 nt or 1000 nt or more of a nucleic acid molecule complementary to a nucleic acid molecule sequence characterized in (a) to (e) and encoding a polypeptide having the activity represented by a protein comprising a polypeptide as depicted SEQ ID NO: 2, 4, 6, 8, 27, or 45, or a homolog thereof.

3. The nucleic acid molecule of claim 2, wherein the nucleic acid molecule according to (a) to (h) is at least one or more nucleotides different from the sequence of SEQ ID 1, 3, 5, 7, 26, or 44 and encodes a protein which differs at least in one or more amino acids from the protein sequence of SEQ ID NO: 2, 4, 6, 8, 27, or 45.

4. A nucleic acid construct which confers the expression of said nucleic acid molecule of claim 2, comprising one or more regulatory elements.

5. A vector comprising the nucleic acid molecule of claim 2.

6. A polypeptide encoded by the nucleic acid molecule of claim 2.

7. A plant cell nucleus, plant cell, plant tissue, propagation material, pollen, progeny, harvested material or a plant, comprising the nucleic acid molecule of claim 2.

8. A plant cell nucleus, a plant cell, a plant tissue, propagation material, seed, pollen, progeny, or a plant part, resulting in a plant with increased herbicide tolerance or resistance after regeneration; or a plant with increased herbicide tolerance or resistance; or a part thereof; wherein said increased herbicide tolerance or resistance is as compared to a corresponding wild type produced by the method of claim 1.

9. The plant cell nucleus, plant cell, plant or part thereof of claim 7, derived from a monocotyledonous plant.

10. The plant cell nucleus, plant cell, plant or part thereof of claim 7, derived from a dicotyledonous plant.

11. The plant cell nucleus, plant cell, plant or part thereof of claim 7, wherein the corresponding plant is selected from the group consisting of corn (maize), wheat, rye, oat, triticale, rice, barley, soybean, peanut, cotton, oil seed rape, including canola and winter oil seed rape, manihot, pepper, sunflower, sugar cane, sugar beet, flax, borage, safflower, linseed, primrose, rapeseed, turnip rape, tagetes, solanaceous plants comprising potato, tobacco, eggplant, tomato; Vicia species, pea, alfalfa, coffee, cacao, tea, Salix species, oil palm, coconut, perennial grass, forage crops and Arabidopsis thaliana.

12. The plant cell nucleus, plant cell, plant or part thereof of claim 7, wherein the corresponding plant is selected from the group consisting of corn, soy, oil seed rape (including canola and winter oil seed rape), cotton, wheat and rice.

13. A transgenic plant comprising one or more of the plant cell nuclei, plant cells, progeny, seed or pollen of claim 7.

14. A transgenic plant, transgenic plant cell nucleus, or transgenic plant cell, comprising one or more of the plant cell nuclei, plant cells, progeny, seed or pollen of claim 7, wherein said transgenic plant, transgenic plant cell nucleus, or transgenic plant cell, is genetically homozygous for a transgene conferring increased herbicide tolerance or resistance as compared to a corresponding non-transformed wild type plant.

15. A process for the identification of a compound conferring increased herbicide resistance or tolerance in a plant, plant cell, or part thereof as compared to a corresponding non-transformed wild type plant cell, a transgenic plant or a part thereof in a plant cell, a transgenic plant or a part thereof, a transgenic plant or a part thereof, comprising the steps:
(a) culturing a plant cell, transgenic plant or a part thereof expressing the polypeptide of claim 6 and a readout system capable of interacting with the polypeptide under suitable conditions which permit the interaction of the polypeptide with said readout system in the presence of a compound or a sample comprising a plurality of compounds and capable of providing a detectable signal in response to the binding of a compound to said polypeptide under conditions which permit the expression of said readout system and of the polypeptide;
(b) identifying if the compound is an effective agonist by detecting the presence or absence or increase of a signal produced by said readout system.

16. A method for the production of an agricultural composition comprising identifying a compound according to the steps of the method of claim 15 and formulating the compound in a form acceptable for an application in agriculture.

17. A composition comprising the nucleic acid molecule of claim 2 and optionally an agriculturally acceptable carrier.

18. (canceled)

19. A polypeptide encoded by SEQ ID 1, 3, 5, 7, 26, or 44.


SEQUENCE LISTING (74 sequences)

SEQ ID NO: 1 - DNA, 1848 bp, Alopecurus
caacgcagacaacagggggacacgacatcgccacaactatgatgatggataaggcctacattgccatcttcttcttcttcactttcgtcttcctgctccgccaaattctacgaggaaagacgagcaatggcgacaacaacaggggcgtgcagctaccgccgagccctccagctatcccgttcctcggccacctccacctcgtggcaaaaaagccgttacacgccacgctgcgcggcctcgccgaccactacgggccgatcttctcgctgcgcctaggcgcgcgtaacgccgtggtggtgtcctccgcggcgtgcgccacggagtgcttcacggagcacgacgtgatattcgccaaccggccccagttcccctcgcagcagctcgtctccttcggcggcacctcgctcatcttctccagctacggcccgcgctggcgcaccctccgccgcgtcgccgccgtgcagctgctctccccgcaccgcgtcgcctgcatgtcaggggttatcgcgtccgagatccgcgcaatgacgcgccggctctgccgcgcggccgccgcaggcgcccgggtccatctgaagcggaggctgttcgagctctccctcagcgtgctcatggagaccatcgccaacaccaagggaacccggccggtggcggacgccgacacggacatgtccatggaggcccaggagttcaagaaggtgatggacgagatcatcccgtacatcggctccgcaaacatgtgggacttcctgccggtgatgcgatggttcgacgtgttcggcgtcaggaacaaaatcctggccgtggtgagcaggagggacgcgttcctgcggcggctcatcgacgccgagcgccagaggctggaagacggcggcggccaaggtgacaagaaaagcatgatcgccgtgctgctcaccttgcagaaaacagagccagaggtgtacactgatactatgatcacgtctctctgtgcgaatttatttggagctggaactgagaccacgtcaaccatgacagaatgggcaatgtcgctcttgctgaaccacccagcagtgatcaagaaggcccaagctgagatcgacgcgtccgtcggaaactcccgtctggttgctgctgacgacgtgccccgcctcgcctacctccaatgcattatcagtgagacgctccgactgtgcccaccggcgccattgctactggcgcacgagtcctccgctgactgcaaggtcggaggatacaacgtgccaagagatactatgcttattgtaagcgcatatgccatacatagggatccggcaacttgggaggatccgacagtgttccgacccgaaaggtttgaggacggcaagggcgacgagatgttggtgataccgtttgggatggggcggcgggggtgccccggggagacgctcgcacggcagatggttgggatggttcttgggacaatgttgcagtgcttcgattgggaacgggtggacagcgtcgaggtggacatgacggaagggggaggggtcaccatgcccaaggccgtacctttggaggctatgtgtagcccgcgtgcatctatgtgtaaagtccttgagaagctctgagcaccgggcttattagtatgtacactagactgatagcgtgcttcgccgcaccgtacgtggttatttggtgtgttcacgcgttgtatgtgtcgttgtttttcaaataatttgatgtattggcaactgtcttacccggaggtgctatcatgcatagggctttgaaagacgtgtaattcgggtgattctgcttttgttattacgattcggttgaattgtttatgctgaacaggatggtcaagatcggaagagcacacgtctg

SEQ ID NO: 2 - protein, 516 aa, Alopecurus
MMMDKAYIAIFFFFTFVFLLRQILRGKTSNGDNNRGVQLPPSPPAIPFLGHLHLVAKKPLHATLRGLADHYGPIFSLRLGARNAVVVSSAACATECFTEHDVIFANRPQFPSQQLVSFGGTSLIFSSYGPRWRTLRRVAAVQLLSPHRVACMSGVIASEIRAMTRRLCRAAAAGARVHLKRRLFELSLSVLMETIANTKGTRPVADADTDMSMEAQEFKKVMDEIIPYIGSANMWDFLPVMRWFDVFGVRNKILAVVSRRDAFLRRLIDAERQRLEDGGGQGDKKSMIAVLLTLQKTEPEVYTDTMITSLCANLFGAGTETTSTMTEWAMSLLLNHPAVIKKAQAEIDASVGNSRLVAADDVPRLAYLQCIISETLRLCPPAPLLLAHESSADCKVGGYNVPRDTMLIVSAYAIHRDPATWEDPTVFRPERFEDGKGDEMLVIPFGMGRRGCPGETLARQMVGMVLGTMLQCFDWERVDSVEVDMTEGGGVTMPKAVPLEAMCSPRASMCKVLEKL

SEQ ID NO: 3 - DNA, 1641 bp, Alopecurus
cacgcttccccaaataattgagacgatacgaaatagtaccatggataaggcggcctacattgccgtcttctgcttcactttcctcttcctgctccaccgcatcctacgaggcagcaagagcaatggcggcaacagcagcaagggcgtgcagctgccgccgagccccccggccatcccgttcctcggccacctccatctcgtggcggagaagccgttgcacgccacgctgcgccgcctcgccgatcgctatgggccggtcttctcgctgcgcctcggcgcgcgcaacgccctggtggtgtccacggcggccggcgccagggagtgcttcacggagcacgacgtgaccttcgccaaccggccccagttcccctcgcagctgctcgtctccttcggcggcacatcgctcatccactccaactacggcccgcgctggcgcatcctccgccgcgtcgccgccgtgcagctgctctccacgcaccgcgtcgcctgcatgtcgggggtcatcgcggctgagatccgcgcaatgacgcgccggctctgccgcgcggccgccgcaggcgcccgggtccatctgaagcggaggctgttcgagctctccctcagcgtgctcatggagaccatcgccaacaccaagggaacccggccggtggcggacgccgacacggacatgtccatggaggcccaggagttcaagaaggtgatggacgagatcatcccgtacatcggctccgcaaacatgtgggacttcctgccggtgatgcgatggttcgacgtgttcggcgtcaggaacaaaatcctggccgtggtgagcaggagggacgcgttcctgcggcggctcatcgacgccgagcgccagaggctggaagacggcggcggccaaggtgacaagaaaagcatgatcgccgtgctgctcaccttgcagaaaacagagccagaggtgtacactgatactatgatcacgtctctctgtgcgaatttatttggagctggaactgagaccacgtcaaccatgacagaatgggcaatgtcgctcttgctgaaccacccagcggtgatcaagaaggcccaagctgagatcgacgcgtccgttggaaactcccgcctggttgctgctgacgacgtgccccgcctcgcctacctccaatgcattatcagtgagacgctccgattgtgcccaccggcgccattgctactggcacacgagtcctccgctgactgcaaggtcggaggatacaacgtgccaagagacacgatgcttattgtaagcgcatatgccatacatagggatccggcaacttgggaggatccgacagtgttccgacctgaaagatttgaggacggcaagggcgacgggatgttggtgataccgtttgggatggggcggcgggggtgccccggggagacgctcgcacggcagatggttgggatggttcttgggacaatgttgcagtgcttcgattgggaacgggtggacggcgtcgaggtggacatgacggaagggggaggggtcaccatgcccaaggccgtacctttggaggctatgtgtagcccgcgtgcatctatgtgtaaagtccttgagaagctctgagcaccgggcttattagtatgtacactagactgatagcgtgcttcgccgcaccg

SEQ ID NO: 4 - protein, 515 aa, Alopecurus
MDKAAYIAVFCFTFLFLLHRILRGSKSNGGNSSKGVQLPPSPPAIPFLGHLHLVAEKPLHATLRRLADRYGPVFSLRLGARNALVVSTAAGARECFTEHDVTFANRPQFPSQLLVSFGGTSLIHSNYGPRWRILRRVAAVQLLSTHRVACMSGVIAAEIRAMTRRLCRAAAAGARVHLKRRLFELSLSVLMETIANTKGTRPVADADTDMSMEAQEFKKVMDEIIPYIGSANMWDFLPVMRWFDVFGVRNKILAVVSRRDAFLRRLIDAERQRLEDGGGQGDKKSMIAVLLTLQKTEPEVYTDTMITSLCANLFGAGTETTSTMTEWAMSLLLNHPAVIKKAQAEIDASVGNSRLVAADDVPRLAYLQCIISETLRLCPPAPLLLAHESSADCKVGGYNVPRDTMLIVSAYAIHRDPATWEDPTVFRPERFEDGKGDGMLVIPFGMGRRGCPGETLARQMVGMVLGTMLQCFDWERVDGVEVDMTEGGGVTMPKAVPLEAMCSPRASMCKVLEKL

SEQ ID NO: 5 - DNA, 1693 bp, Alopecurus
gggggcacacaacacagctcacgcttccccaaataattgagacgatacgaaatagtaccatggataaggcggcctacattgccgtcttctgcttcactttcctcttcctgctccaccgcatcctacgaggcagcaagagcaatggcggcaacagcagcaagggcgtgcagctgccgccgagccccccggccatcccgttcctcggccacctccatctcgtggcggagaagccgttgcacgccacgctgcgccgcctcgccgatcgctatgggccggtcttctcgctccgcctcggcgcgcgcaacgccctggtggtgtccacggcggccggcgccagggagtgcttcacggagcacgacgtgaccttcgccaaccggccccagttcccctcgcagctgctcgtctccttcggcggcacatcgctcatccactccaactacggcccgcgctggcgcaccctccgccgcgtcgccgccgtgcagctgctctccacgcaccgcgtcgcctgcatgtcgggggtcatcgcgtccgagatccgcgcaatgacgcgccggctctgccgcgcggccgccgcaggcgcccgggttcatctgaagcggaggctgttcgagctctccctcagcgtgctcatggagaccatcgcgaataccaaggggacccggccggtggcggacgccgacacggacatgtccttggaggcacaggagttcaagaaggtgatggacgagatcatcccgtatatcggcgcggcaaacatgtgggacttcctgccggtgatgcgttggttcgacgtgttcggcgtcaggaacaaaatcctggccgcggtgagcaggagggacgcgttcctgaggcggctcatcgacgctgagcgccagaggctggaccacggcggcggccaaggtgacaagaaaagcatgatcgccgtgctgctcaccttgcagaaaacagagccagaggtgtacactgatactatgatcactgctctgtgtgcgaatttatttgcagctggaacagagaccacgtcaaccatgacagaatgggcgatgacactcttactgaatcacccagcggtgatcaagaaggcccaagctgagatcgatgggtctgtcggaaactcccgcttggtcgctgccgacgacctgccccgtctcgcctacctccaatgcattatcagtgaggcgctccgactgtatccaccggcgccactgctacttccgcacgagtcctccgccgactgcaaggtcggaggatataatgtgccaagagacacgatgcttatcgtgagtgcatacgccatacatagagatccagcaatttggggggatccgacagtgttccgacctgaaaggtttgaggacggcaagggcgaggggttgttggtgataccgtttgggatgggacggcgggggtgccccggggagacgctcgcacgccagatggttgggatggttcttgggacaatgttgcagtgcttcgattgggaacgggaggacggcatggaggtggacatgactgaggggagagggatcaccatggccaaggccgtgcctttggaggctatgtgtagcccacgtgcaactatgtgtaattttcttgagaagctctgatggttgaattttttgtatgaaaaggtccagcagtctattaataatcatcaacggtggtaaaattatttttaaaggtaatacaaatt

SEQ ID NO: 6 - protein, 515 aa, Alopecurus
MDKAAYIAVFCFTFLFLLHRILRGSKSNGGNSSKGVQLPPSPPAIPFLGHLHLVAEKPLHATLRRLADRYGPVFSLRLGARNALVVSTAAGARECFTEHDVTFANRPQFPSQLLVSFGGTSLIHSNYGPRWRTLRRVAAVQLLSTHRVACMSGVIASEIRAMTRRLCRAAAAGARVHLKRRLFELSLSVLMETIANTKGTRPVADADTDMSLEAQEFKKVMDEIIPYIGAANMWDFLPVMRWFDVFGVRNKILAAVSRRDAFLRRLIDAERQRLDHGGGQGDKKSMIAVLLTLQKTEPEVYTDTMITALCANLFAAGTETTSTMTEWAMTLLLNHPAVIKKAQAEIDGSVGNSRLVAADDLPRLAYLQCIISEALRLYPPAPLLLPHESSADCKVGGYNVPRDTMLIVSAYAIHRDPAIWGDPTVFRPERFEDGKGEGLLVIPFGMGRRGCPGETLARQMVGMVLGTMLQCFDWEREDGMEVDMTEGRGITMAKAVPLEAMCSPRATMCNFLEKL

SEQ ID NO: 7 - DNA, 1668 bp, Alopecurus
ctcaaggtgaaagaagaagactgatcacttccctagctagctcgatcgccacggttaaccatggataagccatacatcgccgtcctctccttcgcccttctgttcctgctccactacgttgtcggcaaggtcagcaatggcaggcgcggcacgaagggcgccgtgcagctgccgccgagccctcgtgccttcccgttcctcggccacctctacgtcctagagaagcccttccacgcatcgctgtgccgcgtcgccgcgcgcctcggcccggtcttctccctgcgcctcggctcccgccgcgccgtggtggtgtgctcgtccgaggccgccagggagtgcttcacggagcacgacgtgaccttcgccgaccggcccaggttcccttcccagctgctcgtctccttcaacggcgccgcgctcgccacgtccagctacggcccgcactggcgcaacctccgccgcgtcgccgccgtgcagctgctctccgcgcaccgcgtcgcctgcatgtccggcgtcatcgccgccgagatccgcgccatggtgcgccggctctgccacacagccgcggcgactcccggcggcgccccgcgggtccagctgaagcggaggctcttcgagctctccctcagcgtgctcatggagaccatcgcgcagaccaaggggacccgttccgaggccgacgccgacacggacatgtctgtggaggcgcaggagttcaagaacgtgacggacaagctcaccccgcacctcggcacggcaaacaagtgggactacctgccggtgttgcggtggttcgacgtgttcggcgtcaggaacaagatcctggccgcggtgggctcgagggacgcgttcctgcggcgactggtcgacgccgaacgccgaaggctggccgacggcggcagcgatggcgacaagaagagtatgatcgctgtgctgctcacgctgcagaagacggaaccggagttctacaccgataccatgatctcggctctctgtgcgaacttgtttggcgctggaacggagaccacatcaaccacgacggagtgggcgatgtcgctgctgctgaaccacccggcggcgctgaagaaggcccaggcagagatcgacgtgtccgtgggtacatcgcgcctggtgtccgccgacgacgtgccccgcctcgcctacctgcagtgcatcgtcagcgagacgctccgcctctacccggcggcgccgctgctgctgccgcaccagtcctccgcggactgcaaggtcggcggctacaacgtgccgagcggcacgatgctgatcgtgaacgcgtacgccatccacagggacccggcggcgtgggaccgcccgctggagttcaggccggagaggttcgaggccgggaaggccgacgggctgttcatgataccgttcgggatggggcggcggaggtgccccggggagacgctagcgctgcggacgatcggcatggtcctcgcaacgctggtgcagtgcttcgactgggaacgggtggacggcgcagaggtggacatgacggagggcggagggctcaccatccccaaggccgtgccgttggaggccgtgtgcaggccgcgcgcagtcatgcacgacgtgcttcagagcctctgacgagcttccgtcttgcagttgtttgtgttcgattggtcatagcatcgtgtgcgg

SEQ ID NO: 8 - protein, 517 aa, Alopecurus
MDKPYIAVLSFALLFLLHYVVGKVSNGRRGTKGAVQLPPSPRAFPFLGHLYVLEKPFHASLCRVAARLGPVFSLRLGSRRAVVVCSSEAARECFTEHDVTFADRPRFPSQLLVSFNGAALATSSYGPHWRNLRRVAAVQLLSAHRVACMSGVIAAEIRAMVRRLCHTAAATPGGAPRVQLKRRLFELSLSVLMETIAQTKGTRSEADADTDMSVEAQEFKNVTDKLTPHLGTANKWDYLPVLRWFDVFGVRNKILAAVGSRDAFLRRLVDAERRRLADGGSDGDKKSMIAVLLTLQKTEPEFYTDTMISALCANLFGAGTETTSTTTEWAMSLLLNHPAALKKAQAEIDVSVGTSRLVSADDVPRLAYLQCIVSETLRLYPAAPLLLPHQSSADCKVGGYNVPSGTMLIVNAYAIHRDPAAWDRPLEFRPERFEAGKADGLFMIPFGMGRRRCPGETLALRTIGMVLATLVQCFDWERVDGAEVDMTEGGGLTIPKAVPLEAVCRPRAVMHDVLQSL

SEQ ID NO: 9 - protein, 517 aa, Alopecurus
MDKAYIAILSCAFLFLVHYVLGKVSDGRRGKKGAVQLPPSPPAVPFLGHLHLVDKPIHATMCRLAARLGPVFSLRLGSRRAVVVSSSECARECFTEHDVTFANRPKFPSQLLVSFNGTALVTSSYGPHWRNLRRVATVQLLSAHRVACMSGVIAAEVRAMARRLFHAAEASPDGAARVQLKRRLFELSLSVLMETIAQTKATRSEADADTDMSVEAQEFKEVVDKLIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTEWAMSLLLNHPAALKKAQAEIDASVGTSRLVSVDDVPSLAYLQCIVNETLRLYPAAPLLLPHESSADCKVGGYNVPADTMLIVNAYAIHRDPAAWEHPLVFRPERFEDGKAEGLFMIPFGMGRRRCPGETLALRTIGMVLATLVQCFDWEPVDGVNVDMTEGGGFTIPKAVPLEAVCRPRAVMRDVLQSI

SEQ ID NO: 10 - protein, 520 aa, Alopecurus
MKYSTSVTMDKAYIAVFSIVILFLLVDYLRRLRGGGTSNGKNKGMRLPPGLPAVPIIGHLHLVKKPMHATLSRLAARHGPVFSLRLGSRRAVVVSSPGCARECFTEHDVAFANRPRFESQLLMSFDGTALAMASYGPHWRNLRRVAAVQLLSARRVGLMSGLIAGEVRAMVRSLCRRPAAAAPVQLKRRLFELSLSVLMETIAQSKATRPETTDTDTDMSMEAQEYKQVVEEILERIGTGNLCDYLPALRWFDVFGVRNRILAAVSRRDAFLRRLIYAARWRMDDGEKKSMIAVLLTLQKTQPEVYTDNMITALCSNLLGAGTETTSTTIEWAMSLLLNHPETLKKAQAEIDASVGNSRLITADDVPRITYLQCIVRETLRLYPAAPMLIPHESSADCEVGGYSVPRGTMLLVNAYAIHRDPAAWEEPERFVPERFEGGGCDGNLSMPFGMGRRRCPGETLALHTVGLVLGTLIQCFDWERVDGVEVDMAEGGGLTMPKVVPLEAVCRPRDAMGGVLREL

SEQ ID NO: 11 - protein, 512 aa, Alopecurus
MDKAYIAVFSIVILFLLVDYLRRLRGGGTSNGKNKGMRLPPGLPAVPIIGHLHLVKKPMHATLSRLAARHGPVFSLRLGSRRAVVVSSPGCARECFTEHDVAFANRPRFESQLLMSFDGTALAMASYGPHWRNLRRVAAVQLLSARRVGLMSGLIAGEVRAMVRSLCRRPAAAAPVQLKRRLFELSLSVLMETIAQSKATRPETTDTDTDMSMEAQEYKQVVEEILERIGTGNLCDYLPALRWFDVFGVRNRILAAVSRRDAFLRRLIYAARWRMDDGEKKSMIAVLLTLQKTQPEVYTDNMITALCSNLLGAGTETTSTTIEWAMSLLLNHPETLKKAQAEIDASVGNSRLITADDVPRITYLQCIVRETLRLYPAAPMLIPHESSADCEVGGYSVPRGTMLLVNAYAIHRDPAAWEEPERFVPERFEGGGCDGNLSMPFGMGRRRCPGETLALHTVGLVLGTLIQCFDWERVDGVEVDMAEGGGLTMPKVVPLEAVCRPRDAMGGVLREL

SEQ ID NO: 12 - protein, 517 aa, Alopecurus
MDKAYIAILSSAFLFLVHYVLGKVSDGRRGKKGAVQLPPSPPAVPFLGHLHLVEKPIHATMCRLAARLGPVFSLRLGSRRAVVVSSSECARECFTEHDVTFANRPKFPSQLLVSFNGTALVTSSYGPHWRNLRRVATVQLLSAHRVTCMSGVIAAEVRAMARRLFHAAEASPDGAARVQLKRRLFELSLSVLMETIAQTKATRSEADADTDMSLEAQEFKEVVDKLIPHLGAANMWDYLPVMRWFDVFGVRSKILHAVSRRDAFLRRLINAERRRLADGGSDGDKKSMIAVLLTLQKTEPKVYTDTMITALCANLFGAGTETTSTTTEWAMSLLLNHPAALKKAQAEIDASVGTSRLVSVDDVPSLAYLQCIVSETLRLYPAAPLLLPHESSADCKVGGYNVPADTMLIVNAYAIHRDPAAWEDPLEFKPERFEDGKAEGLFMIPFGMGRRRCPGETLALRTIGMVLATLVQCFDWEPVDGVKVDMTEGGGFTIPKAVPLEAVCRPRVVMRDVLQNL

SEQ ID NO: 13 - protein, 306 aa, Alopecurus
MSLEAQEFKQVVDEIIPHIGAANLWDYLPALRWFDVFGVRRKILAAVSRRDAFLRRLIDAERRRLDDGDEGEKKSMIAVLLTLQKTEPEVYTDNMITALTANLFGAGTETTSTTSEWAMSLLLNHPDTLKKAQAEIDASVGNSRLITADDVTRLGYLQCIVRETLRLYPAAPMLLPHESSADCKVGGYNIPRGSMLLINAYAIHRDPAVWEEPEKFMPERFEDGGCDGNLLMPFGMGRRRCPGETLALRTVGLVLGTLIQCFDWERVDGVEVDMTEGGGLTIPKVVPLEAMCRPRDAMGGVLRELV

SEQ ID NO: 14 - protein, 516 aa, Alopecurus
MDKTYVAILSFAFLLLLHYLVGRSGGNSNVKKKDVQLPPSPAAIPFLGHLHLVEKPFHAALSRLAARHGPVFSLRLGSRNTVVVSSPACARECFTEHDVSFANRPLFPSQLLVSFNGTALAASSYGPYWRNLRRIATVQLLSAHRVSCMSGVISAEVRAMVLRMYRAAAAAPGSAARILLKRRLLELSLSVLMETIAKTKATRPEADADTDMSVEAQEFKKMSDEIIPQLGTANLWDYLPVLRWFDVFGVRNKVLDAVRRRDAFLRRLIDAERQRLDDGSESEKSSMIAVLLTLQRTEPEVYTDAMITALCGNLFGAGTETISITTEWAMSLLLNHPETLRKAQAEIDASVGSSRLVSADDMPRLSYLQCIVSETLRLYPAAPLLLPHESSTDCKVGGYNIPSGTMLLVNAYAIQRDPTVWEEPTKFKPERFEDGKAEGLFMIPFGMGRRKCPGETLALRTIGLVLGTLIQCFDWDTVDGVEVDMTESGGISMPKAVPLEAICKPRAAMYGVLQNL

SEQ ID NO: 15 - protein, 732 aa, Alopecurus
MAFLGWAVDIARDSGASSSVVLTCDGYGSALYFSPWDSVPLPATASPDDGFLLPRFPDVCVQRSQFTNHLAPANGTGGGGSRTGVKEEASEVLSWPPTSKQSVRRLEVAEHWYRLYKTDNQRLSPDSQQVSVLAESHCDLASGNWKEISIHHKKMPSSTTTKTTTPSRDAWIVSARSDPFHLLLEAQAPLGIKADALSQIAAVHQSHRNTSHIRELSLAMDNAYIIAILSVAILFLLHYYLLGRGNGGAARLPPGPPAVPILGHLHLVKKPMHATMSRLAERYGPVFSLRLGSRRAVVVSSPGCARECFTEHDVTFANRPRFESQLLVSFNGAALATASYGAHWRNLRRIVAVQLLSAHRVGLMSGLIAGEVRAMVRRMYRAAAASPAGAARIQLKRRLFEVSLSVLMETIAHTKATRPETDPDTDMSVEAQEFKQVVDEIIPHIGAANLWDYLPALRWFDVFGVRRKILAAVSRRDAFLRRLIDAERRRLDDGDEGEKKSMIAVLLTLQKTEPEVYTDNMITALTANLFGAGTETTSTTSEWAMSLLLNHPDTLKKAQAEIDASVGNSRLITADDVTRLGYLQCIVRETLRLYPAAPMLLPHESSADCKVGGYNIPRGSMLLINAYAIHRDPAVWEEPEKFMPERFEDGGCDGNLLMPFGMGRRRCPGETLALRTVGLVLGTLIQCFDWERVDGVEVDMTEGGGLTIPKVVPLEAMCRPRDAMGGVLRELV

SEQ ID NO: 16 - protein, 518 aa, Alopecurus
MDKAYIAILSFTFLFLLHYILGKVSNGRRSKGDVQLPPSPRPIPFLGHLHLLEKPFHVALCRLAARLGPVFSLRLGSRRAVVVSSADCARECFTEHDVIFANRPQFPSQLLVSFDGTALSTSSYGPHWRNLRRVAAVQLLSAHRVACMSGVIAGEVRAMARRLFRSAEASPGGGGAARVQLKRRLFELSLSVLMETIAQTKGTRSEADADTDMSVEAQEFKKVVDEIIPYLGAANTWDYLPVVRWFDVFGVRNKILAAVSRRDAFLHRLIDAERRRLDGGGAEADKKSMIAVLLTLQKTEPEVYTDTMITALCSNLFGAGTETTSTTTEWAMSLLLNHPAALRKAQAEIDAAVGTSRLVTADDVPRLAYLQCIVSETLRLYPATPMLLPHQSSADCKVGGYNVPSGTMLMVNAYAIHRDPAAWERPLEFVPERFEDGKAEGRFMIPFGMGRRRCPGETLALRTIGMVLATLVQCFDWDRVDGKEVDMTESGGLTIPKAVPLEAVCRPRAAMRDVLQSL

SEQ ID NO: 17 - protein, 513 aa, Alopecurus
MDNAYIIAILSVAILFLLHYYLLGRGNGGAARLPPGPPAVPILGHLHLVKKPMHATMSRLAERYGPVFSLRLGSRRAVVVSSPGCARECFTEHDVTFANRPRFESQLLVSFNGAALATASYGAHWRNLRRIVAVQLLSAHRVGLMSGLIAGEVRAMVRRMYRAAAASPAGAARIQLKRRLFEVSLSVLMETIAHTKATRPETDPDTDMSVEAQEFKQVVDEIIPHIGAANLWDYLPALRWFDVFGVRRKILAAVSRRDAFLRRLIDAERRRLDDGDEGEKKSMIAVLLTLQKTEPEVYTDNMITALTANLFGAGTETTSTTSEWAMSLLLNHPDTLKKAQAEIDASVGNSRLITADDVTRLGYLQCIVRETLRLYPAAPMLLPHESSADCKVGGYNIPRGSMLLINAYAIHRDPAVWEEPEKFMPERFEDGGCDGNLLMPFGMGRRRCPGETLALRTVGLVLGTLIQCFDWERVDGVEVDMTEGGGLTIPKVVPLEAMCRPRDAMGGVLRELV

SEQ ID NO: 18 - protein, 518 aa, Alopecurus
MDKASIAVLSLAFLFLLHYILGKRSDGRRGKGKGAVQLPPSPPAVPFFGHLHLVEKPLHAALCRLGARHGPVFSLRLGARNAVVVSSPACARECFTDHDVAFANRPQFPSQMLVSYGGTSLVSSSYGPHWRNLRRVAAVRLLSAHRVAGMSGVIAAEVRAMARRLYRAAAASPGGAARVELKRSLFELSLSVLMETIARTKGTRSEADADTDMSLEAQEFKQVVDEIIPLIGAANLWDYLPVMRWFDVSGVRSRILATVSRRDAFLHRLIDAERRRMEEGGDEGEKKSMIAVLLTLQKTEPELYTDQMIIALCANMFVAGTETTSTTIEWAMSLLLNHPAALKKAQAEIDASIGTSRMVAADDVPRLSYLQCIINETLRMYPAAPLLLPHESSADCKVGGYDVPSGTMLIVNAYAIHRDPATWEDPTAFRPERFEDGKGDGLLLMPFGMGRRRCPGEALALQTVGVVLGMLVQCFDWDRVDGVEVDMTEGVGITMPKSVALEAVCRPRAAMRDVLHKL

SEQ ID NO: 19 - protein, 517 aa, Alopecurus
MDKAYIAILSCAFLFLVHYVLGKVSHGRRGKKGAVQLPPSPPAIPFIGHLHLVEKPIHATMCRLAARLGPVFSLRLGSRRAVVVSSSECARECFTEHDVTFANRPKFPSQLLASFNGTALVTPSYGPHWRNLRRVATVQLLSAHRVACMSGVIAAEVRAMARRLFHAAEASPGGAARVQLKRGPFELSLSVLMETIAQTKATRSEADADTDMSVEAQEFKEVVDKPIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTERAMSLLLNHPAALKKAQAEIDASVGTSRLVSVDDMPSLAYLQCIVNETLRLYPAAPLLLPHESSADCKVGGYNVPADTMLIVNAYAIHRDPAAWEHPLEFRPERFEDGKAEGLFMIPFGMGRRRCPGETLALRTIGMVLATLVQCFDWEPVDGVKVDMTEGGGFTIPKAVPLEAVCRPRAVMRDVLQNL

SEQ ID NO: 20 - protein, 517 aa, Alopecurus
MDKAYIAILSCAFLFLVHYVLGKVSHGRRGKKGAVQLPPSPPAIPFIGHLHLVEKPIHATMCRLAARLGPVFSLRLGSRRAVVVSSSECARECFTEHDVTFANRPSSRRKLLASFNGTALVTSSYGPHWRNLRRVATVQLLSAHRVACMSGVIAAEVRAMARRLFHAAEASPDGATRVQLKRRLFELSLSVLMETIAQTKATRSEADADTDMSVEAQEFKEVVDKLIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTEWAMSLLLNHPAALKKAQAEIDASVGTSRLVSVDDVLSLAYLQCIVSETLRLYPAAPLLLPHESSADCKVGGYNVPADTMLIVNAYAIHRDPAAWEHPLEFRPERFEDGKAEGLFMIPFGMGRRRCPGETLALRTIGMVLATLVQCFDWEPVDGVKVDMTEGGGFTIPKAVPLEAVCRPRTVMRDVLQNL

SEQ ID NO: 21 - protein, 517 aa, Alopecurus
MDKAYIAILSCAFLFLVHYVLGKVSDGRRGKKGAVQLPPSPPAIPFIGHLHLVEKPIHATMCRLAARLGPVFSLRLGSRRAVVVPSSECARECFTEHDVTFANRPKFPSQLLASFNGTALVTSSYGPHWRNLRRVATVQLLSAHRVACMSGVIAAEVRAMARRLFHAAEASPDGAARVQLKRRLFELSLSVLMETIAQTKATRSEADADTDMSVEAQEFKEVVDKLIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTEWAMSLLLNHPAALKKAQAEIDASVGTSRLVSVDDVPSLAYLQCIVNETLRLYPAAPLLLPHESSADCKVGGYNVPADTMLIVNAYAIHRDPAAWEHPLEFRPERFEDGKAEGLFMIPFGVGRRRCPGETLALRTISMVLATLVQCFDWEPVDGVKVDMTEGGGFTIPKAVPLEAVCRPRAVMRDVLQNL

SEQ ID NO: 22 - protein, 540 aa, Alopecurus
LTQHTSNQPQTSSVKTRDTSLPMDKAYIAILTIVFLFLLHYILRRVSNGRRGKGAVQLPPSPPAVPFLGHLHLLEKPFHAALGRLAARLGPVFSLRLGSRRAVVVSSAECARECFTEHDVTFANRPRFPSQLLVSFNGAALATSSYGPHWRNLRRVAAVQLLSAHRVACMSGVIAGEVRAMARRLFRAAEASPGGGGAARVQLKRRLFELSLSVLMETIAQTKGTRSEADADTDMSVEAQEFKKVVDEIIPYLGAANTWDYLPVMRWFDVFGVRNKILAAVSRRDAFLHRLIDAERRRLDGGGAEADKKSMIAVLLTLQKTEPEVYTDTMITALCANLFGAGTETTSSTTEWAMSLLLNHPAALRKAQAEIDVAVGTSRLVTADDVPRLAYLQCIVSETLRLYPAAPMLLPHQSSADCKVGGYNVPSGTMLMVNAYAIHRDPAAWERPLEFVPERFEDGKAEGRFMIPFGMGRRRCPGETLALRTIGMVLATLVQCFDWDRVDGKEVDMTESGGLTIPKAVPLEAVCRPRAAMRDVLQSL

SEQ ID NO: 23 - protein, 517 aa, Alopecurus
MDKAYIAILSCAFLFLVHYVLGKVSDGRRGKKGAVQLPPSPPAVPFLGHLHLVDKPIHATMCRLAARLGPVFSLRLGSRRAVVVSSSECARECFTEHDVTFANRPKFPSQLLVSFNGTALVTSSYGPHWRNLRRVATVQLLSAHRVACMSGVIAAEVRAMARRLFHATEASPDGAARVQLKRRLFELSLSVLMETIAQTKATRSEADADTDMSVEAQEFKEVVDKLIPHLGAANMWDYLPVMRWFDVFGVRNKILHAVSRRDAFLRRLIDAERRRLADGGSDGDKKSMIAVLLTLQKTEPKVYTDTMITALCANLFGAGTETTSTTTEWAMSLLLNHPAALKKAQAEIDASVGTSRLVSVDDVPSLAYLQCIVSETLRLYPAAPLLLPHESSADCKVGGYNVPADTMLIVNAYAIHRDPAAWEDPLEFRPERFEDGKAEGLFMIPFGMGRRRCPGETLALRTIGMVLATLVQCFDWEPVDGVKVDMTEGGGFTIPKAVPLEAVCRPRAVMRDVLQNL

SEQ ID NO: 24 - protein, 513 aa, Alopecurus; identical to SEQ ID NO: 17

SEQ ID NO: 25 - protein, 517 aa, Alopecurus
MDKAYIAALSAAALFLLHYLLGRRAGGEGKTKGSQRRLPPSPPAIPFLGHLHLVKAPFHAALARLAARHGPVFSMRLGTRRAVVVSSPDCARECFTEHDVNFANRPLFPSMRLASFDGAMLSVSSYGPYWRNLRRVAAVQLLSAHRVACMAPAIEAQVRAMVRRMDRAAAAGGGGAARVQLKRRLFELSLSVLMETIAHTKTSRAEADADSDMSPEAHEFKQIVDELVPYIGTANRWDYLPVLRWFDVFGVRNKILDAVGRRDAFLRRLIDGERRRLDAGDDSESKSMIAVLLTLQKSEPEVYTDTVITALCANLFGAGTETTSTTTEWAMSLLLNHREALKKAQAEIDAAVGTSRLVTADDVPHLTYLQCIVDETLRLHPAAPLLLPHESAADCTVGGYDVPRGTMLLVNVHAVHRDPAVWDDPDRFVPERFEGGKAEGRLLMPFGMGRRKCPGETLALRTVGLVLGTLLQCFDWDTVDGAQVDMKASGGLTMPRAVPLEAMCRPRTAMRDVLKRL

SEQ ID NO: 26 - DNA, 1799 bp, Alopecurus
gcagtggtatcaacgcagagtacatggggacagcccagtgacccagtctgtgaatccggctatacgatcagacaactcttacgaaacctcagcggcagagccaaagcctgtgttttcctctagttcaccgccccgatgacgatggccacccgagctctccacatactgggtgaggcctctccgtggagcctagccggtgcggcggcggccatggcgctgctgtggctggccgcctggatcctcgagtgggcatggtggaccccgcggcggctgggtcgggccctgcaggctcagggcctcacgggcacccggtaccgcctattcaccggagacgtcacggagaacgcccggctcaacagggcggcccggtccaagcccctgccgctcggctcccacgacatcattcctcgcgtgcagccaatgctcagcaacgccgttaaggagaacgggaaactgtcgttcacttggtttggcccaacaccaagggtgatgattcatgacccagaattagtgagagaaattctgtccaacaagtttggacactacggtaaaccacagactagccgtttgtttaagctgctagccgacgggcttgtcaatcatgaaggcgagaaatgggcaaagcaccggagaatcctaaatcctgcctttcacagtgagaagataaagaggatgctgccagttttttcaacctgtagcgaagaaatgatcacgagatgggagaattcagtgtcctctgaaggattatctgaggtggacgtctggcctgagttccagaatctgactggagatgtcatctcgagaacagcgttcggtagcagttatcaggaggggatgaaaatattccagctccaaggagagctagctgaacggctgatacaagcttttcagacactttttatcccaggctattggttcttaccgactagaaacaacagaagaatgagagcaatcgaccgtgagatctgcacaattctgcgaggaattattgagaagaaaaacagagctattaaaaatggtgatgctagaagcgatgacttgctaggattgctgctggagtcaaatatgcgggaatcaaatgggaaagcagatctaggaatgagcactgaagacacaatggaggaatgcaagctattttattttgcaggcatggagacaacatcagtcttgctcacatggacactaattctgctgagcatgcacccggagtggcaagagcaggcaagaaaggaagtgttgcaccacttcggaagaaccacaccagattttgagaacttgagtcgcctgaaaatagtaactatgattctatatgaagttctcaggctgtacccaccggcagtctttatgaccagaagaacatacaaggcaatggagcttggcggcatcacatatccggcaggagtgaactttatgttgcccgttctctttatccaccatgatcccactatatggggaaaagatgcaagcgaattcaatccacagaggtttgctgatggcatctcgaatgcggcaaagcatccggctgcgttcttcccatttggaggtggtcctcggatctgcatcggccagaactttgcgttactggaagctaagatggctcttagcaccatcctccagcgcttctcgttccagctctcgccgtcctacacccacgctccgtacaccgtgttaaccctccacccgcagcacggtgctccaattatgctgaagaagatatgaccatgcttacatgttgtgtgtaatttgaagttgaagctttgagctgaataaacatagcaccgagtttacatgtgtgtttt

SEQ ID NO: 27 - protein, 527 aa, Alopecurus
MTMATRALHILGEASPWSLAGAAAAMALLWLAAWILEWAWWTPRRLGRALQAQGLTGTRYRLFTGDVTENARLNRAARSKPLPLGSHDIIPRVQPMLSNAVKENGKLSFTWFGPTPRVMIHDPELVREILSNKFGHYGKPQTSRLFKLLADGLVNHEGEKWAKHRRILNPAFHSEKIKRMLPVFSTCSEEMITRWENSVSSEGLSEVDVWPEFQNLTGDVISRTAFGSSYQEGMKIFQLQGELAERLIQAFQTLFIPGYWFLPTRNNRRMRAIDREICTILRGIIEKKNRAIKNGDARSDDLLGLLLESNMRESNGKADLGMSTEDTMEECKLFYFAGMETTSVLLTWTLILLSMHPEWQEQARKEVLHHFGRTTPDFENLSRLKIVTMILYEVLRLYPPAVFMTRRTYKAMELGGITYPAGVNFMLPVLFIHHDPTIWGKDASEFNPQRFADGISNAAKHPAAFFPFGGGPRICIGQNFALLEAKMALSTILQRFSFQLSPSYTHAPYTVLTLHPQHGAPIMLKKI

SEQ ID NO: 28 - protein, 525 aa, Alopecurus
MATRALQMLGEASPWNLACAAAAMALLWLAAWILEWAWWTPRRLGRALEAQGLKGTRYRLFTGDVPENARLNKEARSKPLPLGSHDIIPRVQPMISNAIKENGKLSFTWFGPEPRVTILDPESVREILSNKFGHYGKPRSSRFGKLLANGLVNHQGEKWAKHRRILNPAFHHEKIKRMLPVFSACSEEMITRWENSMSSQGVSEVDVWPEFQNLTGDVISRTAFGSSYQEGTKIFQLQGEQAERLMQAFQTLFIPGYWFLPTKNNRRMRAIDREICTILRGIIEKKDRAIKSGEASSDDLLGLLLESNRRESNGKANLGMSTEDIIEECKLFYFAGMETTSVLLTWTLIVLSMHPEWQEQARKEVLHHFGRTTPDFENLSRLKIVTMVLYEVLRLYPPAIFVTRRTYKAMELGGITYPAGVNLMLPILFIHHDPNIWGKDASEFNPQRFADGISNAVKNPAAFFPFGGGPRICIGQNFALLEAKMALSTILQRFSFELSPSYTHSPYTVLTLHPQHGAPIVLRKI

SEQ ID NO: 29 - protein, 528 aa, Alopecurus
MATLPLLLHLLWEASPWARAGAATAAVVLVWLAAWTLEWAWWTPRRLDRALRAQGLKGTRYRLLTGDVRENARLNREARTKPLPLGSHDIIPRVLPMFHNAVKENGTNSFTWFGPIPRVIIPDPELMREVLSNKFGHFGKPLFSRVGKLLANGLANHEGEKWAKHRRILNPAFHHEKIKGMLPVFATCCADMINRWENSMSSKEPSEMDVWPEFQNLTGDVISRTAFGSNYQEGRNIFQLQGEQAERLIQSFQTIFIPGYWFLPTKNNRRMKEIDREIRKILHGIIRKRERAFIDSEGTNDDLLGLLVESNMRESNGNAKLGMTTEDIIEECKLFYFAGMETTSVLLTWTLILLSMHPEWQEQAREEVLNHFGMGTPDFDNLNRLKIVTMILYEVLRLYPPVVFLSRRTYKEMELGGIKYPSGVSLLLPIIFIHHDPNIWGKDASEFNPQRFEDGISNATKHQAAFFPFGWGPRICIGQNFALLEAKMALSTILQRFSFELSSSYTHAPYTVITLHPQHGAQIRLKKL

SEQ ID NO: 30 - protein, 523 aa, Alopecurus
MLMMLGAASQWILAAAAAAAVAALLWLAVSTLEWAWWTPRRLERALRAQGIRGNRYRLFTGDVPENVRLNREARKKPLPLGCHDIIPRVLPMFSKAVEEHGKPSFTWFGPTPRVMISDPESIREVMSNKFGHYGKPKPTRLGKLLASGVVSYEGEKWAKHRRILNPAFHHEKIKRMLPVFSNCCTEMVTRWENSMSIEGMSEVDVWPEFQNLTGDVISKTAFGSSYEEGRRIFQLQAESAERIIQAFRTIFIPGYWFLPTKNNRRLREIEREVSKLLRGIIGKRERAIKNGETSNGDLLGLLVESNMRESNGKAELGMTTDEIIEECKLFYFAGMETTSVLLTWTLIVLSMHPEWQERAREEVLHHFGRTTPDYDSLSRLKIVTMILYEVLRLYPPVVFLTRRTYKEMELGGIKYPAEVTLMLPILFIHHDPDIWGKDAGEFNPGRFADGISNATKYQTSFFPFGWGPRICIGQNFALLEAKMAICTILQRFSFELSPSYIHAPFTVITLHPQHGAQIKLKKI

SEQ ID NO: 31 - protein, 519 aa, Alopecurus
MLGEAASPWSLAGAGAAVALLWLCAWTLQWAWWTPRRLERALRAQGLRGTRYRLFIGDVAENGRLNREAASRPLPLGSHDVVPRVMPFFCNVLKEHGKLSFVWTGPKPFVIIRDPDLAREILSNKSGNFAKQTTAGIAKFVVGGVVTYEGEKWAKHRRILNPAFHQEKIKRMLPVFLACCTKMITRWVNSMSSEGISELDVWDEFQNLTGDVISRTAFGSSYQEGWRIFQLQEEQAKRVLKAFQRIFIPGYWYLPIENNRRIREIDQEIRTILRGIIVKRDKAVRNGEGSNDDLLGLLVESNMRQSNEKEDVGMSIEDMIEECKLFYAAGSETTSMLLTWTLILLSMHPEWQEQAREEVMHHFGRTTPDHDGLSRLKIVTMILHEVLRLYPPVVFLQRTTHKEIELGGIKYPEGVNFTLPVLSIHHDPSIWGQDAIKFNPERFANGVSKATKFQTAFFSFAWGPRICLGQSFAILEAKMALATILQSFSFELSPSYTHAPHTVLTLQPQYGSPIKLKKL

SEQ ID NO: 32 - protein, 519 aa, Alopecurus
MLIMLGLGLVPAGAAAALAVALVCLAAAAWWTVERAPRRLERALRAQGVGGGRYQLLLGGDVAENGRLNREAWSRPLPLGCHRIAPRVLPLLWNAVRDHGKLSFIWFGPVPRVMIPDPELVREVFNKFDQFGKPKMIRVGKLLATGVVSYEGEKWAKHRRILNHAFHHEKIKRMLPVFANCCTEMVTRWENSISLEAASEIDVWPEFRNLTGDVISRTAFGSSYQEGRRIFQLQEELAQYLTEALQKLFIPGYWYLPTKNNRRMREIDREVRKILLEIIGNKERAITNGENSNDDMLGLLVESNTKQPELRMSTDDIIEECKLFYFAGMETTSVLLTWTLIVLSMHPEWQERAREEVLHHFGRTTTPDYDSLSRLKIVTMILYEVLRLYPPVVLLNRRTFKETNLGGIKFPADMNLILPILFIHHDPEIWGKDASEFNPGRFADGISNASKYHDASFFPFGWGPRICIGQSFALLEAKMALSMILQRFSLELSPSYIHAPYIVLTLRPQHGAQIKLKRI

SEQ ID NO: 33 - protein, 519 aa, Alopecurus
MLGEAASPWSLAGAGAAVALLWLCAWTLQWAWWTPRRLERALRAQGLRGTRYRLFIGDVAENGRLNREAASRPLPLGSHDVVPRVMPFFCNVLKEHGKLSFVWTGPKPFVIIRDPDLAREILSNKSGNFAKQTTAGIAKFVVGGVVTYEGEKWAKHRRILNPAFHQEKIKRMLPVFLACCTKMITRWVNSMSSEGISELDVWDEFQNLTGDVISRTAFGSSYQEGWRIFQLQEEQAKRVLKAFQRIFIPGYWYLPIENNRRIREIDQEIRTILRGIIVKRDKAVRNGEGSNDDLLGLLVESNMRQSNEKEDVGMSIEDMIEECKLFYAAGSETTSMLLTWTLILLSMHPEWQEQAREEVMHHFGRTTPDHDGLSRLKIVTMILHEVLRLYPPVVFLQRTTHKEIELGGIKYPEGVNFTLPVLSIHHDPSIWGQDAIKFNPERFANGISKATKFQTAFFSFAWGPRICLGQSFAILEAKMALATILQSFSFELSPSYTHAPHTVLTLQPQYGSPIKLKKL

SEQ ID NO: 34 - protein, 525 aa, Alopecurus
MATRALQMLREASPWSLAGAAAAMALLWLAAWIVEWAWWTPRRLRRALQAQGLRGTQYRLFTGDVPENARLNREARSKPLPLGSHDIIQRVQPMFSNVIKENGKFSFTWFGPTPRVMIPDPELVREVLSNKFGHYGKQKSSRLGKLLANGLANHQGEKWAKHRRILNPAFHNEKIKRMLPVFATCCEEMITRWDNSMSTEGSSEIDIWPEFQNLTGDVISRTAFGSNYQEGMKIFQLQGELAERLIMAFQTIFIPGYWFLPTKNNKRMRAIDCEIRTILRVIIRKKDKAIKNGEAISDDLLGLLLESNMRESNGKADLGMSTEEIIQECKLFYFAGMETTSVLLTWTLILLSMHPEWQEKARDEVLYHFGRTTPDFEHLSRLKIVTMILYEVLRLYPPITILTRRTYKAMELGGIKYPAGVNLMLPILFIHHDPNLWGKDASEFNPERFADGISNAAKHPGSFFPFGGGPRICIGQNFALLEAKMALSTILQHFSLELSPSYTHAPYTVITLHPQHGAQIRMKKI

SEQ ID NO: 35 - protein, 527 aa, Alopecurus
MQSSGFIPEKGGTSAPSTQVMVVPESNGLTLSDEEYDGLVEQSKRRASDSDQPILQRAEKMKAERNLDNQKTTDRAAQLQKCDEDIKLIKTACRIHETMVMQFFANYGKLSFIWFGPVPRVMIPDPELVREVFNKFDQFGKPKMIRVGKLLATGVVSYEGEKWAKHRRILNHAFHHEKIKRMLPVFANCCTEMVTRWENSISLEAASEIDVWPEFRNLTGDVISRTAFGSSYQEGRRIFQLQEELAQYLTEALQKLFIPGYWYLPTKNNRRMREIDREVRKILLEIIGNKERAITNGENSNDDMLGLLVESNTKQPELRMSTDDIIEECKLFYFAGMETTSVLLTWTLIVLSMHPEWQERAREEVLHHFGRTTTPDYDSLSRLKIVTMILYEVLRLYPPVVLLNRRTFKETNLGGIKFPADMNLILPILFIHHDPEIWGKDASEFNPGRFADGISNASKYHDASFFPFGWGPRICIGQSFALLEAKMALSMILQRFSLELSPSYIHAPYIVLTLRPQHGAQIKLKRI

SEQ ID NO: 36 - protein, 525 aa, Alopecurus
MATRALQMLGEASPWNLACAAAAMAVLWLAAWILEWAWWTPRRLGRALEAQGLKGTRYRLFTGDVPENARLNKEARSKPLPLGSHDIIPRVQPMISNAIKENGKLSFTWFGPEPRVTILDPESVREILSNKFGHYGKPRSSRFGKLLANGLVNHQGEKWAKHRRILNPAFHHEKIKRMLPVFSACSEEMITRWENSMSSQGVSEVDVWPEFQNLTGDVISRTAFGSSYQEGTKIFQLQGEQAERLMQAFQTLFIPGYWFLPTKNNRRMREIDREICTILRGIIEKKDRAIKSGEASSDDLLGLLLESNRRESNGKADLGMSTEDIIEECKLFYFAGMETTSVLLTWTLIVLSMHPEWQEQARKEVLHHFGRTTPDFENLSRLKIVTMVLYEVLRLYPPAIFVTRRTYKAMELGGITYPAGVNLMLPILFIHHDPNIWGKDASEFNPQRFADGISNAVKHPAAFFPFGGGPRICIGQNFALLEAKMALSTILQRFSFELSPSYTHAPYTVLTLHPQHGAPIVLRKI

SEQ ID NO: 37 - protein, 525 aa, Alopecurus
MATRVLLMLRETSPWALAGAAASVALLWLVAWTLEWAWWTPRRLDRALRAQGLKGTRYRLFTGDLRETARVNREARKNPLPLGCHDIAPRVQPMLHSAMKEYGKLSFTWFGPTPRVMIPDPELVKEVLSNKFGHFGKPRSSRIGKLLANGVVNHDGEKWAKHRRILNPAFHHEKIKRMLPVFSTCCIETIIRWENSMPSEGSSEIDVWPEFQNLTGDVISRTAFGSNYQEGRRIFQLQGELAERLIQSIQTIFIPGYWFLPTKNNRRMKEIDLEIRKILREIIGKREKATRNGETNNDDLLGLLLESNTRQSNGNASLGLTTEDVIEECKLFYFAGMETTSVLLTWTLIVLSMHPEWQERAREEVLSHFGRTRPDFDSLSRLKIVTMILHEVLRLYPPATFLTRRTYKEMELGGIKYPAGVNLLLPIIFIHHDPDIWGKDASEFNPERFANGISNATRHQAAFFPFGGGPRICIGQSFALLEAKMALCTILQRFSFELSPSYTHAPYTVITLHPQHGAQIRLKKL

SEQ ID NO: 38 - protein, 538 aa, Alopecurus
MPLPFLCFSSTAPMVTSVLLILRETSPWALVGAAASVALLWLVAWTLEWAWWTPRRLDRALRAQGLKGTKYRLLTGDVRENARLNREARTKPLALGSHDIIPRVLPMLHNVVKEYGTNSFTWFGPVPRVIIPDPELVREVLSNKFGHFGKPRFSRLGKLLANGLANHEGEKWAKHRRILNPAFHHEKIKRMLPVFATCCTDMINRWENSMSSEGSSEIDVWPEFQNLTGDVISRTAFGSNYQEGRNIFQLQGEQAERLIQSFQTIFIPGYWFLPTKNNRRMKEIDREICKVLHGIIRKRERAFIDGEGSNDDLLGLLVESNMRESNGNAKLGMSTKDIIEECKLFYFAGMETTSVLLTWTLIVLSMHPEWQERARDEVLNHFGRGRPDFDSLNRLKIVTMILYEVLRLYPPVILLTRRTYKEMELGGITYPSGVSLLLPIIFIHHDPNIWGKDASEFNPQRFEDGISNATKHQAAFFPFGWGPRICIGQNFALLEAKMALCTILQRFSFELSPSYTHAPYTVITLHPQHGAQIRLKKL

SEQ ID NO: 39 - protein, 525 aa, Alopecurus
MATRALQMLREASPWSLAGAAAAMALLWLAAWIVEWAWWTPRRLRRALQAQGLRGTQYRLFTGDVPENARLNREARSKPLPLGSHDIIQRVQPMFSNVIKENGKFAFTWFGPTPRVMIPDPELVREVLSNKFGHYGKQKSSRLGKLLANGLANHQGEKWAKHRRILNPAFHNEKIKRMLPVFATCCEEMITRWDNSMSTQGSSEIDIWPEFQNLTGDVISRTAFGSNYQEGMKIFQLQGELAERLIMAFQTIFIPGYWFLPTKNNKRMRAIDCEIRTILRGIIGKKDKAIKNGEAISDDLLGLLLESNMRESNGKADLEMSTEEIIQECKLFYFAGMETTSVLLTWTLILLSMHPEWQEKARDEVLYHFGRTTPDFEHLSRLKIVTMILYEVLRLYPPITILTRRTYKAMELGGIKYPAGVNLMLPILFIHHDPNLWGKDASEFNPERFADGISNAAKHPGSFFPFGGGPRICIGQNFALLEAKMALSTILQHFSLELSPSYTHAPYTVITLHPQHGAQIRIKKI

SEQ ID NO: 40 - protein, 527 aa, Alopecurus
MATCVLLMLREVSPWALASVVASVSLLWLVVWTLEWAWWTPWRLERALRVQGLKGTRYRLFTGDLRETARANREARKKPLPLGSHDIAPRVQPMHHSTIKEYGKLSFTWFGPTPRVMIPDPELVKEVLSNKFGHFGKPRSNRIGRLLANGLVNHDGEKWAKHRRILNPAFHHEKIKGMMPVFSTCCIEMITRWDNSMPSEGSSEIDVWPEFQNLTGDVISRTAFGSNYQEGRRIFELQGELAERLIQSVQTIFIPGYWFLPTKNNRRMRAIDVEIRKILREIIGKREKDTKNRETNNDDLLGLLLESNTRQSNGNASLGLTTEDVIEECKLFYFAGMETTSVLLTWTLIVLSMHPEWQERAREEVLSHFGRTTPDYDSLSRLKTITMILHEVLRLYPPATFLTRRTYKEMELGGIKYPAGVDLLLPVIFIHHDPDIWGKDASEFNPERFANGISSATRHQAAFFPFGGGPRICIGQSFALLEAKMTLCTILQRFSFELSPSYTHAPYTVITLHPQHGAQIRLKKLSP

SEQ ID NO: 41 - protein, 528 aa, Alopecurus
MATLPLLLHLLWEASPWARAGAATAAVVLVWLAAWTLEWAWWTPRRLDRALRAQGLKGTRYRLLTGDVRENARLNREARTKPLPLGSHDIIPRVLPMFHNAVKENGTNSFTWFGPIPRVIIPDPELMREVLSNKFGHFGKPLFSRVGKLLANGLANHEGEKWAKHRRILNPAFHHEKIKRMLPVFATCCADMINRWENSMSSKEPSEMDVWPEFQNLTGDVISRTAFGSNYQEGRNIFQLQGEQAERLIQSFQTIFIPGYWLLPTKNNRRMKEIDREIRKILHGIIRKRERAFIDSEGTNDDLLGLLVESNMRESNGNAKLGMTTEDIIEECKLFYFAGMETTSVLLTWTLILLSMHPEWQEQAREEVLNHFGMGTPDFDNLNRLKIVTMILYEVLRLYPPVVFLSRRTYKEMELGGIKYPSGVSLLLPIIFIHHDPNIWGKDASEFNPQRFEDGISNATKHQAAFFPFGWGPRICIGQNFALLEAKMALSTILQRFSFELSSSYTHAPYTVITLHPQHGAQIRLKKL

SEQ ID NO: 42 - protein, 525 aa, Alopecurus
MATRALQMLGEASPWNLACAAAAMAVLWLAAWILEWAWWTPRRLGRALEAQGLKGTRYRLFTGDVPENARLNKEARSKPLPLGSHDIIPRVQPMISNAIKENGKLSFTWFGPEPRVTILDPESVREILSNKFGHYGKPRSSRFGKLLANGLVNHQGEKWAKHRRILNPAFHHEKIKRMLPVFSACSEEMITRWENSMSSQGVSEVDVWPEFQNLTGDVISRTAFGSSYQEGTKIFQLQGEQAERLMQAFQTLFIPGYWFLPTKNNRRMRAIDREICTILRGIIEKKDRAIKSGEASSDDLLGLLLESNRRESNGKADLGMSTEDIIEECKLFYFAGMETTSVLLTWTLIVLSMHPEWQEQARKEVLHHFGRTKPDFENLSRLKIVTMVLYEVLRLYPPAIFVTRRTYKAMELGGITYPAGVNLMLPILFIHHDPNIWGKDASEFNPQRFADGISNAVKHPAAFFPFGGGPRICIGQNFALLEAKMALSTILQRFSFELSPSYTHAPYTVLTLHPQHGAPIVLRKI

SEQ ID NO: 43 - protein, 523 aa, Alopecurus; identical to SEQ ID NO: 30

SEQ ID NO: 44 - DNA, 1668 bp, Alopecurus
gcgtgtcgtgcagactgcagagtccccgctcctgagagaaccaccatgtccacgggcctcgtctggatggtggcggcggccatcgcggcggtgctggcgacgtgggcgttcaacgcgctggtgcgcctcgtgtggaggccgcgcgccatcaccaggcagctccgcgcgcagggcgtgggcgggccggcgtacaagctcttcgccgggaacctcggcgagatcaagcagctccgcgccgagaccgccggcgccgcgctggacgtcggctcccacgacttcgtccccctcgtgcagccgcacttccgcaaatggatccccattcacggacgcacgttcctgtactggttcggcgcgaggccgaccctgtgcatcgccgacgtgaacgtggtgaaacaggtcctcttcgaccgcaacgggctctaccccaagaacaccggcaacccgcacatcgcccgcctgctcggcaaggggctcgtgctcatcgacggcgacgactggaagcgccaccgcaaggtcgtccacccggccttcaacatggacaagctcaagatgatgacggtgaccatgtccgactgcgctgggtcaatgatgtcggagtggaaagccaagctggagaagggcggcgaagcggagattgacctcagcaggcagtttgaggagctaaccgcggacgtgatctcccacacggcgttcggcagcagctacacggaggggaaaaaggtctttctggcgcagagggacctccagtttctggcattttccactgtattcagcgtccaaatcccagcattcaggtacattccgacccaaaagaaccgtcagatatggaagctcgacagggaggtgaggaccatgctcaccaacatcatcaaaacccggctcgcgaccaaagacaccatgggctacggaaacgacctgctcgggctcatgctggaggcgtgcgcgccagagcacggggagactccgattctgagcatggacgagatcatcgacgagtgcaagaccttcttcttcgccgggcacgacaccagctcgcacctgctcacgtggaccatgttcttgctgagcacgcacccggagtggcaggagaagctcagggaggaggtgctgacagagtgtggcaatgaggttcccaccggcgacatgctcaacaagctcaagttggtcaacatgttcctactggaaactctcaggttatactcccctgtgtccgtaattcagaggaagacaggttcagatatggaggtcggtggcatcaaagtgccccaaggaactgtcctgaccatccccatcgcgacgatgcatcgtgacaaggaggtctggggtgaggatgcggatgaattcaagcctatgaggttcgagaaaggtgtgaccatggctgccaagcaccccaatgccttgttgtctttctccagcgggccgaggtcgtgcatagggcagaacttcgcgatgatcgaagccaaggctgtgatcgctgtgattcttcagaggttctccttctccctgtcccctaagtacgtccatgcaccgatggacgtgatcacgctgcggcccaagtttggtcttcccatggtcctgaagagcctggagatgtagagacatgcatacagtgtattcaggttgagtaacacgaagtacttcactagggtttacattactagattgtac

SEQ ID NO: 45 - protein, 516 aa, Alopecurus
MSTGLVWMVAAAIAAVLATWAFNALVRLVWRPRAITRQLRAQGVGGPAYKLFAGNLGEIKQLRAETAGAALDVGSHDFVPLVQPHFRKWIPIHGRTFLYWFGARPTLCIADVNVVKQVLFDRNGLYPKNTGNPHIARLLGKGLVLIDGDDWKRHRKVVHPAFNMDKLKMMTVTMSDCAGSMMSEWKAKLEKGGEAEIDLSRQFEELTADVISHTAFGSSYTEGKKVFLAQRDLQFLAFSTVFSVQIPAFRYIPTQKNRQIWKLDREVRTMLTNIIKTRLATKDTMGYGNDLLGLMLEACAPEHGETPILSMDEIIDECKTFFFAGHDTSSHLLTWTMFLLSTHPEWQEKLREEVLTECGNEVPTGDMLNKLKLVNMFLLETLRLYSPVSVIQRKTGSDMEVGGIKVPQGTVLTIPIATMHRDKEVWGEDADEFKPMRFEKGVTMAAKHPNALLSFSSGPRSCIGQNFAMIEAKAVIAVILQRFSFSLSPKYVHAPMDVITLRPKFGLPMVLKSLEM

SEQ ID NO: 46 - protein, 460 aa, Alopecurus
MVAAAAAAVLASWAFSAVVHLVWRPHAISRRLRAQGVGGPGYRFFSGNLGEIKRFRGDGAGVVLNVSSHDFLPIVQPHFRKWISLYDRTGIYPKNLTNSHFVRLLGKGLVLTDGDEWKRHRKVVHPAFNMDKLKMMTMTMSDCSRSMMSEWESELAAKGGLVEIELSRRFEELTADVISHTAFGSSYKEGKQLPSDHEKLFKTWSLDKKVRGMLMDIIKTRHANKNVAWYGNDLLGLMLEACAPEHGESCPQLSMDEIIDECKTFFFAGHDTTSHLLTWTMFLLSTHPDWQEKLREEIAMECGDKVPAGDMLNKLKMVNMFLLETLRLYSPVSLIRRKVGTDIELGGIKMPEGALLTIPIATIHRDKEVWGEDADEFRPERFENGVTRAAKHPNALLSFSSGPRSCIGQNFAMIEAKAVIAMILQRFSFTLSPKYVHAPTDVITLRPKYGLPMILKSLKL

SEQ ID NO: 47 - protein, 528 aa, Alopecurus
MGLAWMVTAAVAAVLASWAFNALVHLVWRPYAITRRLRAHGVRGPPYTFFTGSLGEIKRLRAKGATVTLDVDDHDFIPMVQPHLRKWIALYGRTFVYWTGARPNVCVADVNVVRQVLFDRTGLYPKNLMNPHISRLLGKGLVLTDGNDWKRHRKVVHPAFNMDKLKLMTATMSDCARSMISEWDAQLQKEESGRDGHGHGHVEEELSSRFEELTADVISHTAFGSSYSEGKRVFLAQRELQHIAFSTIFNVQIPALKYLPTEKNLRTRKLDRQVRAMLMDIIEARLASKDTAGGYGNDLLGLMLEACAPPPEHHGEMAPTTLSMDEIVDECKTFFFAGHDTTSHLLTWASFLLSTHPEWQHRLRDEVRRECGDDEVPTGDALNRLKLVNMFLLETLRLYGPVSLIQRKAGSDLDLGGIRVPEGAILTIPIATIHRDKEVWGEDAGEFRPERFENGVTRAAKHPNALLSFSSGPRSCIGQNFAMIEAKAVVAMILQRFALELSPKYVHAPMDLITLRPRHGLPMLLKRL

SEQ ID NO: 48 - protein, 517 aa, Alopecurus
MGPAWMVASAVAAVLASWAFNALVRLVWRPYAVARRLRAQGVRGPAYRFLAGNLAEMKRLRAEGARATLDVGDHDFVPMVQPHHRKWISLYGRTFLYWNGATPNLCLADVNLVRQVLFDRTGLYPKNHINQYVTRLLGRGLLLTDGDEWKRHRKVVHPAFNMDKLKTMTATMSDCALSMISEWEAKLAKGGDAEVVELSSQFEELTADVISRTAFGSSYREGRQVFLAQRELQFLAFSTAFDVQIPALRYLPTRNNLRTRELDRRVRGMLMDIIKARLAVAGKDTAGGGYGHDLLGLMLEAAEHGGEAPTLSMDEIVDECKTFFFAGYDTTSHLLTWACFLLSTHPEWQGRLREEVRQECGADEVPTGDALNRLRLVNMFLLETLRLYGPVSLIQRKAGTDLDLGGVRVPEGAILTIPIATIHRDTEVWGDDAGEFRPERFQNGVTRAAKHPNALLAFSSGPRSCIGQNFAMIEAKAVVAIILQRFALELSPTYVHAPMDVITLRPRHGLPMLLRSL

SEQ ID NO: 49 - protein, 514 aa, Alopecurus
MDLAWMVAAAVASVLASWAFNALLHLVWRPYAITRSLRAQGVRGPDYRFLTGNLAEMKRLRADGAAVTLDVGDHDFIPMVQPHHRKWISLYGRTFVYWNGATPNVCLADVNVVRQVLFDRTGLYPKNLMNPHVSRLLGKGLVLTDGDDWKRHRKVVHPAFNMDKLKMMTATMSNCALSMMSEWEAQLAKGAGDAEVELSTRFEELTADVISHTAFGSSYEDGKRVFLAQRELQFLAFSTFFNVQIPALRYLPTEKNRRTWKLDKQVRGMLMDIIKARVANKDTAGYGNDLLGLMLEACAPEHGETPVLSMDEIIDECKTFFFAGHDTTSHLLTWAAFLLSTHPEWQDRLREEVRRECGDEVPTRGDALNKLALVNMFLLETLRLYGPVSLIQRKAGSDLDLGGIRVPEGAIFTIPIATIHRDKEVWGDDAGEFKPERFENGVTRAAKHPNALLSFSSGPRSCIGQNFAMIEAKAVVAMILQRFALELSPKYVHAPMDVITLRPRHGLPMLLRRL

SEQ ID NO: 50 - protein, 491 aa, Alopecurus
MAMGFLAWMVAAAAAAVLASWAFSAVVHLVWRPRAISRRLRAQGVGGPGYRFFSGNLGEIKRFRGDGAGVVLNVSSHDFLPIVQPHFRKWIPLYGRTFLYWFGAQPNICLADVSMVWQVLSDRTGIYPKNLTNPHFVRLLGKGLVLTDGDEWKRHRKWESELAAKGGLVEIELSRRFEELTADVISHTAFGSSYKEGKQVFLAQRELQFLAFSTFLTVQIPGFSYLPTMKNFKTWSLDKKVRGMLMDIIKTRHANKDVAGYGNDLLGLMLEACAPEHGESCPQLSMDEIIDECKTFFFAGHDTTSHLLTWTMFLLSTHPDWQEKLREEIAMECGDKVPTGDMLNKLKMVNMFLLETLRLYSPVSLIRRKVDTDIELGGIKMPEGALLTIPIATIHRDKEVWGEDADEFRPERFENGVTRAAKHPNALLSFSSGPRSCIGQNFAMIEAKAVIAMILQRFSFTLSPKYVHAPTDVITLRPKYGLPMILKSLKL

SEQ ID NO: 51 - protein, 514 aa, Alopecurus
MGLVWLVAAAVAVVLASWAFNALVYLVWRPRAITRQLRAQGVGGPGYRFFAGNLAEIKQLRADSAGAALDIGNHDFVPRVQPHFRKWIPIHGRTFLYWFGARPSLCVADVNTVKQVLSDRSGLYPKSIGNPHIARLLGKGLVLTDGDDWKRHRKVVHPAFNMDKLKMMTVTMSDCAGSMMSEWKAKMDKGGSVEIDLSHQFEELTADVISHTAFGSSYEQGKKVFLAQRELQFLAFSTVFNVQIPAFRYLPTEKNVKIWKLDKEVRTMLMNIIKGRLATKDIMGYGNDLLGLMLEACAPEDRQNPLLSMDEIIDECKTFFFAGHDTSSHLLTWTMFLLSTHPKWQEKLREEVLRECGNGVPTGDMLNKLQLVNMFLLETLRLYAPVSAIQRKAGSDLEVGGIKVPEGTVLTIPIATIHRDKEVWGEDANEFKPMRFENGVTRAGKHPNALLSFSSGPRSCIGQNFAMIEAKAVIAVILQRFSFSLSPKYVHAPMDVITLRPKFGLPMVLKSLEM

SEQ ID NO: 52 - protein, 521 aa, Alopecurus
MGNFVWMVAAAAAAVASWAFIAVVVKLVWRPRAISRRLRAQAVGGPGYRFFSGNLGEIRRLRAEGAGVVLDVSSHDFVPIVQPHFRKWVSLYGKTFLFWFGAQPNICLADINIVRQVLSDRTGMYPKDLTNPYFAHLLGKGLVLIDGDEWKRHYKVVHPAFDMDKLKMMTVTISDCTGSMMSEWESELGMKGGSAEIELSQRFQELTADVISRTAFGSSYSEGKQVFLAQRKLQFLAFSMFLTIQIPGFRYLPTKKNLKIWSLDKKVRSMLRNIIKIRLANKDTMGYGNDLLGLMLETCAPEHDESQQLSMDEIIAECKTFFFGGHDTTSHLLTWTMFLLSTHPEWMRKIRKEVTTMCGDEVPTGDMLNKMNLLNMFLLETLRLYSPVSLISRRTGTNAKFGGIKVPEGTILRIPIATIHRDKEVWGEDADEFKPARFENGVSKAAKHPNALLSFSNGPRSCIGQNFAMIEAKAVITMILQRFSFTLSPKYVHTPISVITLRPKYGLPMILRSLKLYKIGM

SEQ ID NO: 53 - protein, 529 aa, Alopecurus
MGLAWMVAAAVAAVLASWAFNALVHLVWRPYAITRRLRAQGVRGPPYTFFTGSLGEIKRLRGEGAAVTLDVDDHDFIPMVQPHLRKWIALYGRTFVYWTGARPNVCVADVNVVRQVLFDRTGLYPKNLMNPHISRLLGKGLVLTDGDDWKRHRKVVHPAFNMDKLKLMTATMSDCARSMISEWDAQLQKEESGRDGHGHGHVEVELSSRFEELTADVISHTAFGSSYSEGKRVFLAQRELQHIAFSTIFNVQIPALKYLPTKKNVRTRKLDRQVRAMLMGIIEARLASKDTAGGYGNDLLGLMLEACAPPPEHHGEMALTTLSMDEIVDECKTFFFAGHDTTSHLLTWATFLLSTHPEWQHRLRDEVRRECGDDDEVPTGDALNRLKLVNMFLLETLRLYGPVSLIQRKAGSDLDLGGIRVPEGAILTIPIATIHRDKEVWGEDAGEFRPERFENGVTRAAKHPNALLSFSSGPRSCIGQNFAMIEAKAVVAMILQRFALELSPKYVHAPMDLITLRPRHGLPMLLKRL

SEQ ID NO: 54 - protein, 512 aa, Alopecurus
MDLAWMVAAAVAAVLASWAFNALVHLVWRPYAITRSLRAQGVRGPDYRFFTGSLGEIKRLRGEGAAVTLDVDDHDFIPMVQPHLRKWIALYGRTFVYWTAARPNVCVADVNVVRQVLFDRTGLYPKNLMNPHVSRLLGKGLVLTDGDDWKRHRKVVHPAFNMDKLKMMTVTMSDCARSMMSEWEAQLAKGGEVEVELSSRFEELTADVISHTAFGSSYNEGKQVFLAQRELQYIAFSTVFNVQIPALKYLPTEKNLKTRKLDRQVRGMLMDIIKARLTSKDTAGYGNDLLGLMLEACAPEHGETPVLSMDEIIDECKTFFFAGHDTTSHLLTWASFLLSTHPEWQDRLREEVRRECGDEVPTGDALNKLKLVNMFLLETLRLYGPVSLIQRKAGSDLDLGGIRVPEGAILTIPIATIHRDKEVWGDDAGEFKPERFENGVTRAAKHPNALLSFSSGPRSCIGQNFAMIEAKAVVAMILQRFALELSPKYVHAPMDVITLRPRHGLPMLLKRL

SEQ ID NO: 55 - protein, 520 aa, Alopecurus
MGNFVWMVAAAAAAVASWAFIAVVVKLVWRPRAISRRLRAQAVGGPGYRFFSGNLGEIRRLRAEGAGVVLDVSSHDFVPIVQPHFRKWVSLYGKTFLFWFGAQPNICLADINIVRQVLSDRTGMYPKDLTNPYFAHLLGKGLVLIDGDEWKRHYKVVHPAFDMDKLKMMTVTISDCTGSMMSEWESELGMKGGSAEIELSQRFQELTADVISRTAFGSSYSEGKQVFLAQRKLQFLAFSMFLTIQIPGFRYLPTKKNLKIWSLDKKVRSMLRNIIKIRLANKDTMGYGNDLLGLMLETCAPEHDESQQLSMDEIIAECKTFFFGGHDTTSHLLTWTMFLLSTHPEWMRKIRKEVTTMCGDEVPTGDMLNKMNLLNMFLLETLRLYSPVSLISRRTGTNAKFGGIKVPEGTILRIPIATIHRDKEVWGEDADEFKPARFENGVSKAAKHPNALLSFSNGPRSCIGQNFAMIEAKAVITMILQRFSFTLSPKYVHTPISVITLRPKYGLPMILRSLKVKRDL

SEQ ID NO: 56 - protein, 509 aa, Alopecurus
MGFEWTVATAAAAVARRGRSSRWWRLGAQGVAGPGYRFFSGNLSEIRRLRAEGANLVLDVSSHDFVPIVQPHIRTWIPLYGKTFLYWFGTRPNICLADMNMVRQVLSDRTGMFPKYIDNMQFARLLGKGLVLTDDDEWKRHYKVVHPAFDMDKLKMMTETISDCARSMMFEWESELGMKGGSTEIELSRWFEELTVDVISRTAFGSIYREGKQVFLAQRKLQFLAFSAFLTIQIPGFSYLLTKKNMKTWSLDKKVRSMLMNIIKSRLTNKETMGYGNDLLGLMLEACVPEHGGSQPQLSMDDIIAECKTFFFAGHDTTSQLLTWTMFLLSTHQHWMEKLRKEVRMVCNDEVPTGDMLNKLKLVNMFLLETLRLYGPVSLVTRRDGTDVKLGSIKVPKGTILTIPIATIHRDKEVWGEDADEFKPERFENGVLKAAKHPSALLSFSIGLRSCIGQNFAMIEAKTIIAMILQRFSFTLSPKYVHTPISVITLRPKYGLPMILRSLKVKRDR

SEQ ID NO: 57 - protein, 517 aa, Alopecurus
MGNLGWMVAAAVAAVVASWAFDAVVKLVWRPRAITRRLRAQGVGGPGYRFFSGNLGEIRRLRDEGAGVVLDVSSHDFVPIVQPHFRKWIPLYGKTFMYWFGARPTICLADVSMVRQVLSDRTGMYPKNVSNPYFARLLGKGLVLTDGDEWKRHRKVVHPAFNMDKLKMMTVTMSDCAQSMISEWESELGTKGDIVEIELSRRFEELTADVISHTAFGSSYKEGKQVFLAQRELQFLAFSTFLSIQIPGSSYLPTKKNLKTWSVDKKVRSMLTDIIKSRLNNKDVAGYGNDLLGLMLEACAPEHGESQPQLSMDEIIAECKTFFFAGHDTTSHLLTWTMFLLSTHPEWQEKLREEVATECDGKVPTGDMLNKLKLVNMFLLETLRLYGPVAFIQRRVNAELELGGITVPEGTVLSIPIATIHRDKEVWGEDADIFKPERFKNGVSKAGKYPNALLSFSSGPRACIGQNFAMIEAKAVIAMILQRFSFTLSPKYVHVPTDVITLRPKYGLPMILKSLKV

SEQ ID NO: 58 - protein, 502 aa, Alopecurus
MGFEWTVATAAAAVAASRLGAQGVAGPGYRFFSGNLSEIRRLRAEGANLVLDVSSHDFVPIVQPHIRKWIPLYGKTFLYWFGTRPNICLADMNMVRQVLSDRTGMFPKYIDNMQFARLLGKGLVLTDDDEWKRHYKVVHPAFDMDKLKMMTETISDYAQSMMFEWESELGMKGGSTEIELSRWFEELTADVISRTAFGSSYREGKQVFLAQRKLQFLAFSVFLTIQIPGFSYLLTKKNLKTWSLDKKVRSMLMNIIKSRLTNKETMGYGNDLLGLMLEACVPEHGGSQPQLSMDDIIAECKTFFFAGHDTTSQLLTWTMFLLSTHQHWMEKLRKEVRMVCNDEVPTGDMLNKLKLVNMFLLETLRLYGPVSLVTRRAGTDVKLGSIKVPKGTILTIPIATIHRDKEVWGEDADEFKPERSENGVLNAAKHPSALLSFSIGLRSCIGQNFAMIEARTIIAMILQRFSFTLSPKYVHTPISVITLRPKYGLPMILRSLKVKRDR

SEQ ID NO: 59 - protein, 520 aa, Alopecurus
MGNFVWMVAAAAAAVASWAFIAVVVKLVWRPRAISRRLRAQAVGGPGYRFFSGNLGEIRRLRAEGAGVVLDVSSHDFVPIVQPHFRKWVSLYGKTFLFWFGAQPNICLADINIVRQVLSDRTGMYPKDLTNPYFAHLLGKGLVLIDGDEWKRHYKVVHPAFDMDKLKMMTVTISDCTGSMMSEWESELGMKGGSAEIELSQRFQELTADVISRTAFGSSYSEGKQVFLAQRKLQFLAFSMFLTIQIPGFRYLPTKKNLKIWSLDKKVRSMLTNIIKIRLANKDTMGYGNDLLGLMLETCAPEHDESQQLSMDEIIAECKTFFFGGHDTTSHLLTWTMFLLSTHPEWMRKIRKEVTTMCGDEVPTGDMLNKMNLLNMFLLETLRLYGPVSLISRRTGTNAKFGGIKVPEGTILRIPIATIHRDKEVWGEDADEFKPARFENGVSKAAKHPNALLSFSNGPRSCIGQNFAMIEAKAVITMILQRFSFTLSPKYVHTPISVITLRPKYGLPMILRSLKVKRDL

SEQ ID NO: 60 - protein, 519 aa, Alopecurus
MAMGLLAWMVAAAAAAVLASWAFSAVVHLVWRPRAISRRLRAQGVGGPGYRFFSGNLGEIKRFRGDGAGVVLNVSSHDFLPIVQPHFRKWIPLYGRTFLYWFGAQPNICLADVSMVWQVLSDRTGIYPKNLTNPHFVRLLGKGLVLTDGDEWKRHRKVVHPAFNMDKLKMMTMTMSDCSRSMMSEWESELAAKGGLVEIELSRRFEELTADVISHTAFGSSYKEGKQVFLAQRELQFLAFSTFLTVQIPGFSYLPTMKNFKTWSLDKKVRGMLMDIIKTRHANKDVAGYGNDLLGLMLEACAPEHGESCPQLSMDEIIDECKTFFFAGHDTTSHLLTWTMFLLSTHPDWQEKLREEIAMECGDKVPTGDMLNKLKMVNMFLLETLRLYSPVSLIRRKVDTDIELGGIKMPEGALLTIPIATIHRDKEVWGEDADEFRPERFENGVTRAAKHPNALLSFSSGPRSCIGQNFAMIEAKAVIAMILQRFSFTLSPKYVHAPTDVITLRPKYGLPMILKSLKL

SEQ ID NO: 61 - protein, 514 aa, Alopecurus
MGLLWMAAAAVAAVLASWAFNALVHLVWRPRAITRQLRAQGVGGPGYSFFAGNLGEIKRLLADTAGAVLDVGDHDFVPRVQPHFRKWIPIHGRTFLYWFGARPTLCVADVNVVKQVLADRSGMYPKNVGNPHIARLLGKGLVLTDGDDWKRHRKVVHPAFNMDKLKMMTMTMSDCAGSMMSEWKAKMEKGGNMEIELSRQFEELTADVISHTAFGSSYQQGKKVFLAQRELQFLAFSTVFNVQIPAFRYLPTEKNLKIWKLDKEVRGMLMNIIKTRLDTKDTMGYGNDLLGLMLEACALEHGQNPILSMDEIIDECKTFFFAGHDTSSHLLTWTMFLLSMHPEWQEKLREEVLRECGNGAPTGDMLNKLHLVNMFLLETLRLYGPVAAIQRKAGSDLEVGGIKVPKGTVITIPIATIHRDKEVWGEDANEFKPMRFENGVTRAGKHPNALLSFSSGPRSCIGQNFAMIEAKAVIAMILQRFSFSLSPKYVHAPMDVITLRPKFGLPMILKSLEM

SEQ ID NO: 62 - protein, 401 aa, Alopecurus
MNPHISRLLGKGLVLTDGDDWKRHRKVVHPAFNMDKLKLMTATMSDCARSMISEWDAQLQKEESGRDGHGHGHVEVELSSRFEELTADVISHTAFGSSYSEGKRVFLAQRELQHIAFSTIFNVQIPALKYLPTKKNVRTRKLDRQVRAMLMGIIEARLASKDTAGGYGNDLLGLMLEACAPPPEHHGEMALTTLSMDEIVDECKTFFFAGHDTTSHLLTWATFLLSTHPEWQHRLRDEVRRECGDDDEVPTGDALNRLKLVNMFLLETLRLYGPVSLIQRKAGSDLDLGGIRVPEGAILTIPIATIHRDKEVWGEDAGEFRPERFENGVTRAAKHPNALLSFSSGPRSCIGQNFAMIEAKAVVAMILQRFALELSPKYVHAPMDLITLRPRHGLPMLLKRL

SEQ ID NO: 63 - protein, 491 aa, Alopecurus
PRAITRRLGAQGVAGPGYRFFSGNLSEIRRLRAEGANLVLDVSSHDFVPIVQPHIRKWIPLYGKTFLYWFGTRPNICLADMNMVRQVLSDRTGMFPKYIDNMQFARLLGKGLVLTDDDEWKRHYKVVHPAFDMDKLKMMTETISDYAQSMMFEWESELGMKGGSTEIELSRWFEELTADVISRTAFGSSYREGKQVFLAQRKLQFLAFSVFLTIQIPGFSYLLTKKNLKTWSLDKKVRSMLMNIIKSRLTNKETMGYGNDLLGLMLEACVPEHGGSQPQLSMDDIIAECKTFFFAGHDTTSQLLTWTMFLLSTHQHWMEKLRKEVRMVCNDEVPTGDMLNKLKLVNMFLLETLRLYGPVSLVTRRAGTDVKLGSIKVPKGTILTIPIATIHRDKEVWGEDADEFKPERSENGVLNAAKHPSALLSFSIGLRSCIGQNFAMIEARTIIAMILQRFSFTLSPKYVHTPISVITLRPKYGLPMILRSLKVKRDR

SEQ ID NO: 64 - protein, 337 aa, Alopecurus
MSEWESELGAKGGLAEIELSRRFEELTADVISHTAFGSSYKEGKQVFLAQRELQFLAFSTFLTVQILGYSYLLTMKNFKTWSLDKKVRGMLMDIIKTRHANKDVVGYGNDLLGLLLEACAPEHGESHPQLSMDEIIDECKTFFFAGHDTTSHLLTWTMFLLSTHPDWQEKLREDIAMECGDEVPTGDMLNKLKMVNMFLLETLRLYSPVLLIRRKVGTDIELGGIKMPEGALLTIPIATIHRDKEVWGEDADEFRLERFENGVTRAAKHPDALLSFSSGPRSCIGQNFAMIEAKAVIAMILQRFSFTLSPKYVHAPTDVITLRPKYGLPMILKSLKL

SEQ ID NO: 65 - protein, 514 aa, Alopecurus
MGLVWMVAAAVVAVLASWAFNALVHLVWRPRAITRQLRAQGVGGPGYRFFAGNLGEIKRLRDDTAGAALDVGDHDFVPMVQPHFRKWIPIHGRTFLYWFGARPSLCVADVNVVKQVLADRNGMYPKNIGNPHIARLLGKGLVLTDGDDWKRHRKVVHPAFNMDKLKMMTVTMSECAGSMMSEWETKMDKGGSVEIDLSTQFEEITADVISHTAFGSSYEQGKKVFLAQRELQFLAFSTVFSVQIPAFRYLPTEKNLKIWKLDKEVRTMLMNIIESRLATKDTMGYGNDLLGLMLEACAAEGGHTPILSMDEIIDECKTFFFAGHDTSSHLLTWTVFLLSTHPEWQEKLREEVLRECGSEVPTGDMLNKLHLVNMFLLETLRLYAPVSLIQRKAGSDLEVGGIKVPEGTVLTIPIATIHRDKEVWGEDANEFKPMRFENGVARAGKHPNALLSFSSGPRSCIGQSFAMIEAKAVIAVILQRFSFSLSPKYVHAPMDVITLRPKFGLPMILKSIEI

SEQ ID NO: 66 - protein, 514 aa, Alopecurus
MGLVWMVAAAVAAVLASWAFDALVYLVWRPRAITRQLRAQGVGGPGYRFFAGNLAEIKQLRADSAGAALDIGDHDFVPRVQPHFRKWIPIHGRTFLYWFGAKPTLCIADVNVVKQVLSDRGGLYPKSIGNPHIARLLGKGLVLTDGDDWKRHRKVVHPAFNMDKLKMMTVTMSDCAGSMMSEWKAKMDKGGSVEIDLSSQFEELTADVISHTAFGSSYEQGKKVFLAQRELQFLAFSTVFNVQIPSFRYLPTEKNLKIWKLDKEVRTMLMNIIKGRLATKDTMGYGNDLLGLMLEACAPEDGQNPLLSMDEIIDECKTFFFAGHDTSSHLLTWTMFLLSTHPEWQEKLREEVLRECGNGIPTGDMLNKLQLVNMFLLETLRLYAPVSAIQRKAGSDLEVGGIKVTEGTFLTIPIATIHRDKEVWGEDANKFKPMRFENGVTRAGKHPNALLSFSSGPRSCIGQNFAMIEAKAVIAVILQRFSFSLSPKYVHAPMDVITLRPKFGLPMILKSLEM

SEQ ID NO: 67 - protein, 454 aa, Alopecurus
NAMSTNYFNLLAYIYIYIIVCLLCVVGKTFLFWFGAQPNICLADINIVRQVLSDRTGMYPKDLTNPYFAHLLGKGLVLIDGDEWKRHYKVVHPAFDMDKLKMMTVTISDCTGSMMSEWESELGMKGGSAEIELSQRFQELTADVISRTAFGSSYSEGKQVFLAQRKLQFLAFSMFLTIQIPGFRYLPTKKNLKIWSLDKKVRSMLRNIIKIRLANKDTMGYGNDLLGLMLETCAPEHDESQQLSMDEIIAECKTFFFGGHDTTSHLLTWTMFLLSTHPEWMRKIRKEVTTMCGDEVPTGDMLNKMNLLNMFLLETLRLYSPVSLISRRTGTNAKFGGIKVPEGTILRIPIATIHRDKEVWGEDADEFKPARFENGVSKAAKHPNALLSFSNGPRSCIGQNFAMIEAKAVITMILQRFSFTLSPKYVHTPISVITLRPKYGLPMILRSLKVKRDL

SEQ ID NO: 68 - protein, 517 aa, Alopecurus
MGNLGWMVAAAVAAVVASWAFDAVVKLVWRPRAITRRLRAQGVGGPGYRFFSGNLGEIKRLRDEGAGVVLDVSSHDFVPIVQPHFRKWIPLYGKTFMYWFGARPTICLADVSMVRQVLSDRTGMYPKNVSNPYFARLLGKGLVLTDGDEWKRHRKVVHPAFNMDKLKMMTVTMSDCAQSMISEWESELGTKGDIVEIELSRRFEELTADVISHTAFGSSYKEGKQVFLAQRELQFLAFSTFLSIQIPGSSYLPTKKNLKTWSVDKKVRSMLTDIIKSRLNNKDVAGYGNDLLGLMLEACAPEHGESQPQLSMDEIIAECKTFFFAGHDTTSHLLTWTMFLLSTHPEWQEKLREEVAMECDGKVPTGDMLNKLKLVNMFLLETLRLYGPVAFIQRRVNAELELGGITVPEGTVLSIPIATIHRDKEVWGEDADIFKPERFENGVSKAGKYPNALLSFSSGPRACIGQNFAMIEAKAVIAMILQRFSFTLSPKYVHAPTDVITLRPKYGLPMILKSLKV

SEQ ID NO: 69 - protein, 512 aa, Alopecurus
MDLAWMVAAAVASVLASWAFNALVHLVWRPYAITRSLRAQGVRGPDYRFFTGSLGEIKRLRAEGAAVTLDVDDHDFIPMVQPHLRKWIALYGRTFVYWTGARPNVCVADVNVVRQVLFDRTGLYPKNLMNPHVSRLLGKGFVLTDGDDWKRHRKVVHPAFNMDKLKMMTVTMSDCARSMMSEWEAQLAKGGEVEVELSSRFEELTADVISHTAFGSSYNEGKQVFLAQRELQYIAFSTVFNVQIPVFRYLPTEKNLKTRKLDRQVRGMLMDIIKTRLASKDTAGYGNDLLGLMLEACAPEHGETPVLSMDEIIDECKTFFFAGHDTTSHLLTWASFLLSTHPEWQDRLREEVRRECGDEVPTGDALNKLKLVNMFLLETLRLYGPVSLIQRKAGSDLDLGGIRVPEGAILTIPIATIHRDKEVWGDDAGEFKPERFENGVTRAAKHPNALLSFSSGPRSCIGQNFAMIEAKAVVAMILQRFALELSPKYVHAPMDVITLRPRHGLPMLLKRL

SEQ ID NO: 70 - DNA, 1567 bp, Alopecurus
ggcgcgccaccatgatgatggataaggcttatatcgctatattcttcttctttacattcgtgttcttgctcaggcagatcctcagaggaaagacctctaacggagataacaacaggggagtgcagttgcctccatctcctccagctattcctttcctcggacatctccatctcgtggctaagaaacctctccacgctactcttagaggactcgctgatcactacggacctatcttctcacttagactcggagctagaaacgctgtggtggtttcttctgctgcttgtgctactgagtgcttcactgagcacgatgtgatcttcgctaacaggcctcagttcccatctcagcagcttgtttctttcggaggaacctctctcatcttctcatcttacggacctaggtggaggaccctcagaagagttgctgctgttcagctcttgtctcctcatagagtggcttgcatgtctggtgtgatcgcttctgagatcagggctatgactagaaggctctgtagagctgctgctgctggtgctagagttcatctcaagagaaggcttttcgagctttctctctctgtgctcatggaaactatcgctaacaccaagggaaccaggcctgttgctgatgctgataccgatatgtctatggaagctcaagagttcaagaaagtgatggatgagatcatcccttacatcggatctgctaatatgtgggatttcttgcctgtgatgagatggttcgatgtgttcggagtgaggaacaagatcctcgctgtggtgtctagaagggatgctttccttagaaggctcatcgatgctgagagacagagacttgaagatggtggtggacagggtgataagaaatctatgatcgctgtgctcctcaccctccaaaagactgaacctgaggtgtacaccgataccatgatcacttcactctgcgctaacctcttcggagctggaactgagactacttctactatgaccgagtgggctatgtctctcctccttaaccatcctgctgtgatcaagaaggctcaggctgagatcgatgcttctgtgggaaactctagactcgtggctgctgatgatgttcctagacttgcttacctccagtgcatcatctctgagactcttagactttgccctcctgctccacttctcttggctcatgaatcttcagctgattgcaaggtgggaggatacaacgtgccaagagataccatgctcatcgtgtctgcttacgctatccacagagatcctgctacttgggaggatcctactgtgttcagacctgagagattcgaggatggaaagggtgatgagatgctcgtgatccctttcggaatgggaagaagaggttgtcctggagagactctcgctagacagatggttggaatggtgctcggaactatgctccagtgtttcgattgggagagagtggattctgttgaagtggatatgaccgagggtggtggtgtgactatgcctaaggctgttcctcttgaggctatgtgctcaccaagagcttctatgtgcaaggtgctcgagaagctccctgcagg

SEQ ID NO: 71 - DNA, 1564 bp, Alopcurus
ggcgcgccaccatggataaggctgcttacattgctgtgttctgctttaccttcttgttcttgctccacaggatcctcaggggatctaagtctaacggaggaaactcttctaagggagttcagctccctccatctcctccagctattcctttccttggacatctccatctcgtggctgagaagcctcttcatgctactcttagaaggctcgctgataggtacggacctgtgttctctcttagactcggtgctagaaacgctctcgtggtttctactgctgctggtgctagagaatgcttcactgagcacgatgtgaccttcgctaacagacctcagttcccatctcagctcctcgtttctttcggaggaacttctctcatccactctaactacggacctaggtggaggatcctcagaagagttgctgctgttcagttgctctctacccatagagtggcttgcatgtctggtgtgatcgctgctgaaatcagggctatgactagaaggctctgtagagctgctgcagctggtgcaagagttcatctcaagagaaggcttttcgagctttctctctctgtgctcatggaaactatcgctaacaccaagggaaccaggcctgttgctgatgctgataccgatatgtctatggaagctcaagagttcaagaaagtgatggatgagatcatcccttacatcggatctgctaatatgtgggatttcttgcctgtgatgagatggttcgatgtgttcggagtgaggaacaagatcctcgctgtggtgtctagaagggatgctttcttgagaaggctcatcgatgctgagagacagagacttgaagatggtggtggacagggtgataagaaatctatgatcgctgtgctcctcaccctccaaaagactgaacctgaggtgtacaccgataccatgatcacttcactctgcgctaacctcttcggagctggaactgagactacttctactatgaccgagtgggctatgtctctcctccttaaccatcctgctgtgatcaagaaggctcaggctgagatcgatgcttctgtgggaaactctagactcgtggctgctgatgatgttcctagacttgcttacctccagtgcatcatctctgagactcttagactttgccctcctgctccacttctcttggctcatgaatcttcagctgattgcaaggtgggaggatacaacgtgccaagagataccatgctcatcgtgtctgcttacgctatccacagagatcctgctacttgggaggatcctactgtgttcagacctgagagattcgaggatggaaagggtgatggaatgctcgtgatccctttcggaatgggaagaagaggttgtcctggagagactctcgctagacagatggttggaatggtgctcggaactatgctccagtgtttcgattgggagcgtgtggatggtgtggaagtggatatgactgagggtggtggtgtgactatgcctaaggctgttcctcttgaggcaatgtgctcaccaagagcttctatgtgcaaggtgctcgagaagctccctgcagg

SEQ ID NO: 72 - DNA, 1564 bp, Alopecurus
ggcgcgccaccatggataaggctgcttacattgctgtgttctgctttaccttcttgttcttgctccacaggatcctcaggggatctaagtctaacggaggaaactcttctaagggagttcagctccctccatctcctccagctattcctttccttggacatctccatctcgtggctgagaagcctcttcatgctactcttagaaggctcgctgataggtacggacctgtgttctctcttagactcggtgctagaaacgctctcgtggtttctactgctgctggtgctagagaatgcttcactgagcacgatgtgaccttcgctaacagacctcagttcccatctcagctcctcgtttctttcggaggaacttctctcatccactctaactacggacctaggtggagaaccctcagaagagttgctgctgttcagttgctctctacccatagagtggcttgcatgtctggtgtgatcgcttctgagatcagggctatgactagaaggctctgtagagctgctgcagctggtgcaagagttcatctcaagagaaggcttttcgagctttctctctctgtgctcatggaaactatcgctaacaccaagggaaccaggcctgttgctgatgctgataccgatatgtctctcgaggctcaagagttcaagaaagtgatggatgagatcatcccttacatcggagctgctaatatgtgggatttcttgcctgtgatgagatggttcgatgtgttcggagtgaggaacaagatcctcgctgctgtgtctagaagggatgctttcttgagaaggctcatcgatgctgagagacagagactcgatcatggtggtggacagggtgataagaaatctatgatcgctgtgctcctcaccctccaaaagactgaacctgaggtgtacaccgataccatgatcactgctttgtgcgctaacctcttcgctgctggaactgagactacttctactatgaccgagtgggctatgaccctccttcttaaccatcctgctgtgatcaagaaggctcaggctgagatcgatggatctgtgggaaactctagactcgtggctgctgatgatcttcctagacttgcttacctccagtgcatcatctctgaggctcttagactttaccctccagctccacttctcttgcctcatgagtcatctgctgattgcaaggtgggaggatacaacgtgccaagagataccatgctcatcgtgtctgcttacgctatccacagggatcctgctatttggggagatcctactgtgttcagacctgagagattcgaggatggaaagggtgagggacttctcgttatccctttcggaatgggaagaagaggttgccctggagagactcttgctagacagatggttggaatggtgctcggaactatgctccagtgttttgattgggagagggaagatggtatggaagtggatatgaccgagggaaggggtatcactatggctaaggctgttcctcttgaggctatgtgctctcctagagctactatgtgcaacttcctcgagaagctccctgcagg

SEQ ID NO: 73 - DNA, 1600 bp, Alpecurus
ggcgcgccaccatgactatggctactagagcacttcatatcctcggagaggcttctccttggtctttggctggtgctgctgctgctatggctcttttgtggcttgctgcttggattcttgaatgggcttggtggactcctagaaggcttggtagagcattgcaagctcagggactcactggaaccagatacagactcttcactggtgatgtgaccgagaacgctagactcaacagagctgctagatctaagcctctccctctcggatctcacgatatcatccctagagttcagcctatgctctctaacgctgtgaaagagaacggaaagctctctttcacctggttcggacctactcctagagtgatgatccacgatcctgagcttgtgagagagatcctctcaaacaagttcggacactacggaaagcctcagacctctagacttttcaagctcctcgctgatggactcgtgaatcatgaaggtgagaagtgggctaagcacagaaggatccttaaccctgctttccactctgagaagatcaagaggatgctccctgtgttctctacctgctctgaagagatgatcaccaggtgggagaactctgtgtcatctgagggactcagtgaagtggatgtttggcctgagttccagaacctcaccggtgatgtgatctctagaaccgctttcggatcttcttaccaagagggaatgaagatcttccagctccagggtgaacttgctgagagacttatccaggctttccagacccttttcatccctggatactggttcctcccaaccagaaacaacagaaggatgagggctatcgatagagagatctgcaccatcctcaggggaatcatcgagaagaagaacagggctatcaagaacggtgatgctaggtctgatgatctccttggattgctcctcgagtctaacatgagagagtctaacggaaaggctgatctcggaatgtctaccgaggataccatggaagagtgcaagcttttctacttcgctggaatggaaactacctctgtgctccttacttggaccctcatcctcctttcaatgcaccctgaatggcaagagcaggctagaaaagaggtgctccatcacttcggaagaaccacccctgatttcgagaacctctctaggctcaagatcgtgaccatgatcctctacgaggtgctcagactttaccctcctgctgtgttcatgactagaaggacctacaaggctatggaactcggaggaatcacttaccctgctggtgtgaacttcatgctccctgttctcttcatccaccacgatcctactatctggggaaaggatgcttctgagttcaaccctcagaggttcgctgatggaatctcaaacgctgctaagcaccctgctgcattcttcccttttggtggtggacctagaatctgcatcggacagaacttcgctctcctcgaagctaagatggctctctctactatcctccagaggttcagtttccagctctctccttcttacacccacgctccatacactgtgcttactcttcatcctcagcacggtgctcctattatgctcaagaagatccctgcagg

SEQ ID NO: 74 - DNA, 1567 bp, Alopecurus
ggcgcgccaccatgtctactggacttgtttggatggttgctgctgctatcgctgctgttcttgctacttgggctttcaacgctctcgtgagattggtttggaggcctagagctatcactagacagcttagggctcaaggtgttggaggacctgcttacaagcttttcgctggaaacctcggagagatcaagcagttgagagctgaaactgctggtgctgctctcgatgtgggatctcatgatttcgttcctctcgtgcagcctcacttcagaaagtggattcctatccacggaaggacattcctctactggtttggagctagacctaccctctgtatcgctgatgttaacgtggtgaagcaggttctcttcgataggaacggactctaccctaagaacaccggaaaccctcatatcgctagactcctcggaaagggactcgttctcatcgatggtgatgattggaagaggcacagaaaggtggtgcaccctgctttcaacatggataagctcaagatgatgaccgtgaccatgtctgattgcgctggatctatgatgtctgagtggaaggctaagctcgagaagggtggtgaagctgagatcgatctttctaggcagttcgaggaactcaccgctgatgtgatctctcacactgctttcggatcttcatacaccgagggaaagaaggtgttcctcgctcagagagatcttcagttcctcgctttctctaccgtgttctctgttcagatccctgctttcagatatatccctacccagaagaacaggcagatctggaagctcgatagagaagtgagaaccatgctcaccaacatcatcaagaccaggctcgctaccaaggataccatgggatacggaaacgatctcctcggacttatgcttgaggcttgtgctcctgaacatggtgagactcctatcctctctatggatgagatcatcgatgagtgcaagaccttcttcttcgctggacacgatacctcttctcacctccttacctggactatgttcctcctctcaactcaccctgagtggcaagagaagctcagagaagaggttctcactgagtgcggaaacgaggttccaactggtgatatgctcaacaagctcaagctcgtgaacatgttcttgctcgagactctcaggctctactctcctgtttctgtgatccagagaaagaccggatctgatatggaagtgggaggaatcaaggtgccacagggaactgttctcactatccctatcgctaccatgcacagggataaggaagtttggggagaggacgctgatgagttcaagcctatgaggtttgagaagggtgtgaccatggctgctaagcaccctaacgctttgctctcattctcttcaggacctaggtcttgcatcggacagaacttcgctatgatcgaggctaaggctgtgatcgctgttatcctccagaggttctctttctcactctctccaaagtacgtgcacgcacctatggacgtgataaccctcagacctaagttcggactccctatggttctcaagtctctcgagatgcctgcagg